(19) United States
(12) Patent Application Publication
(10) Pub. No.: US 2024/0382726 A1
(43) Pub. Date: Nov. 21, 2024

(54) BALLOON DILATION INSTRUMENT WITH DILATION CATHETER ACTUATOR

(71) Applicants: Acclarent, Inc., Irvine, CA (US); Biosense Webster (Israel) Ltd., Yokneam (IL)

(72) Inventors: Brittanie Chu, Chino Hills, CA (US); Alex Gonzalez, Lake Forest, CA (US); Cesar Fuentes-Ortega, Pasadena, CA (US); Sheila Ruiz, Mission Viejo, CA (US); Henry F. Salazar, Pico Rivera, CA (US); George Gusein, Acre (IL); Hao H. Tran, Santa Ana, CA (US); Zachary Radner, Anaheim, CA (US)

(21) Appl. No.: 18/596,887

(22) Filed: Mar. 6, 2024

Related U.S. Application Data
(60) Provisional application No. 63/467,673, filed on May 19, 2023.
(60) Provisional application No. 63/526,339, filed on Jul. 12, 2023.
(60) Provisional application No. 63/538,093, filed on Sep. 13, 2023.
(60) Provisional application No. 63/611,229, filed on Dec. 18, 2023.
(60) Provisional application No. 63/467,679, filed on May 19, 2023.
(60) Provisional application No. 63/467,683, filed on May 19, 2023.

(51) Int. Cl. A61M 29/02; A61M 25/01

(52) U.S. Cl. CPC A61M 29/02; A61M 25/0136; A61M 2029/025; A61M 2210/0618; A61M 2210/065; A61M 2210/0675

(57) ABSTRACT

An apparatus includes a handle, a guide member extending distally from the handle, and a dilation catheter slidably disposed relative to the guide member. The dilation catheter is operable to translate relative to the handle along a longitudinal range of motion from a proximal-most position to a distal-most position. The dilation catheter includes an expandable element configured to dilate a passageway within the head of a patient and a distal end. The dilation catheter actuator includes a rotary member operable to rotate relative to the handle to thereby drive translation of the dilation catheter along the longitudinal range of motion.

### PRIORITY

This application claims priority to U.S. Provisional Pat. App. No. 63/467,673, entitled “Balloon Dilation Instrument with Dilation Catheter Actuator,” filed May 19, 2023, the disclosure of which is incorporated by reference herein, in its entirety.

This application also claims priority to U.S. Provisional Pat. App. No. 63/526,339, entitled “Balloon Dilation Instrument with Dilation Catheter Actuator,” filed Jul. 12, 2023, the disclosure of which is incorporated by reference herein, in its entirety.

This application also claims priority to U.S. Provisional Pat. App. No. 63/538,093, entitled “Balloon Dilation Instrument with Dilation Catheter Actuator,” filed Sep. 13, 2023, the disclosure of which is incorporated by reference herein, in its entirety.

This application also claims priority to U.S. Provisional Pat. App. No. 63/611,229, entitled “Balloon Dilation Instrument with Dilation Catheter Actuator,” filed Dec. 18, 2023, the disclosure of which is incorporated by reference herein, in its entirety.

This application also claims priority to U.S. Provisional Pat. App. No. 63/467,679, entitled “Guide Rail Actuation Assembly for Balloon Dilation Instrument,” filed May 19, 2023, the disclosure of which is incorporated by reference herein, in its entirety.

This application also claims priority to U.S. Provisional Pat. App. No. 63/467,683, entitled “Balloon Dilation Instrument with Translating Guide Tip Element,” filed May 19, 2023, the disclosure of which is incorporated by reference herein, in its entirety.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pat. No. 11,534,192, entitled “Methods and Apparatus for Treating Disorders of the Sinuses,” issued Dec. 27, 2022, the disclosure of which is incorporated by reference herein, in its entirety; U.S. Pat. No. 9,579,448, entitled “Balloon Dilation Catheter System for Treatment and Irrigation of the Sinuses,” issued Feb. 28, 2017, the disclosure of which is incorporated by reference herein, in its entirety; U.S. Pat. No. 9,155,492, entitled “Sinus Illumination Lightwire Device,” issued Oct. 13, 2015, the disclosure of which is incorporated by reference herein, in its entirety; and U.S. Pub. No. 2021/0361912, entitled “Shaft Deflection Control Assembly for ENT Guide Instrument,” published Nov. 25, 2021, the disclosure of which is incorporated by reference herein, in its entirety.

In the context of Eustachian tube dilation, a dilation catheter or other dilation instrument may be inserted into the Eustachian tube and then be inflated or otherwise expanded to thereby dilate the Eustachian tube. The dilated Eustachian tube may provide improved ventilation from the nasopharynx to the middle ear and further provide improved drainage from the middle ear to the nasopharynx. Methods and devices for dilating the Eustachian tube are disclosed in U.S. Pat. No. 10,206,821, entitled “Eustachian Tube Dilation Balloon with Ventilation Path,” issued Feb. 19, 2019, the disclosure of which is incorporated by reference herein, in its entirety; and U.S. Pat. No. 11,013,896, entitled “Method and System for Eustachian Tube Dilation,” issued May 25, 2021, the disclosure of which is incorporated by reference herein, in its entirety.

Some medical instruments may include an adjustable guide that allows the same medical instrument to readily access different anatomical structures (e.g., Eustachian tubes and different passageways associated with drainage of paranasal sinuses, etc.). Examples of dilation instruments with adjustable guides are described in U.S. Pat. No. 10,137,285, entitled “Balloon Dilation System with Malleable Internal Guide,” issued Nov. 27, 2018, the disclosure of which is incorporated by reference herein, in its entirety; U.S. Pat. No. 11,013,897, entitled “Apparatus for Bending Malleable Guide of Surgical Instrument,” issued May 25, 2021, the disclosure of which is incorporated by reference herein, in its entirety; and U.S. Pat. No. 11,534,192, entitled “Methods and Apparatus for Treating Disorders of the Sinuses,” issued Dec. 27, 2022, the disclosure of which is incorporated by reference herein, in its entirety.

In some scenarios, it may be desirable to allow a dilation catheter of a medical instrument to translate longitudinally relative to a guide of the same instrument. This may allow the guide to be initially positioned in relation to a targeted anatomical passageway while the dilation catheter is in a proximal position. The dilation catheter may then be advanced relative to the guide to a distal position to thereby enter the targeted anatomical passageway. While several systems and methods have been made and used to dilate anatomical passageways within a patient, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a perspective view of an example of a medical instrument, with a dilation catheter in a proximal position;

FIG. 2A depicts a side elevation view of the instrument of FIG. 1, with a housing removed, and with the dilation catheter in the proximal position;

FIG. 2B depicts a side elevation view of the instrument of FIG. 1, with a housing removed, and with the dilation catheter in a distal position;

FIG. 3A depicts a perspective view of another example of a medical instrument, with a dilation catheter in a proximal position;

FIG. 3B depicts a perspective view of the instrument of FIG. 3A, with the dilation catheter in a distal position;

FIG. 4 depicts a rear elevation view of a dilation catheter actuator of the instrument of FIG. 3A;

FIG. 5 depicts a front elevation view of the dilation catheter actuator of FIG. 4;

FIG. 6 depicts a top plan view of the dilation catheter actuator of FIG. 4;

FIG. 7 depicts a cross-sectional side view of the dilation catheter actuator of FIG. 4;

FIG. 8 depicts a perspective view of a handle body of the instrument of FIG. 3A;

FIG. 9 depicts another perspective view of the handle body of FIG. 8;

FIG. 10A depicts a side cross-sectional view of the instrument of FIG. 3A, with the dilation catheter in the proximal position;

FIG. 10B depicts a side cross-sectional view of the instrument of FIG. 3A, with the dilation catheter in the distal position;

FIG. 11 depicts a perspective view of another example of a dilation catheter actuator that may be incorporated into a dilation instrument;

FIG. 12 depicts a front elevation view of the dilation catheter actuator of FIG. 11;

FIG. 13 depicts a rear elevation view of the dilation catheter actuator of FIG. 11;

FIG. 14 depicts a side elevation view of the dilation catheter actuator of FIG. 11;

FIG. 15 depicts a cross-sectional side view of the dilation catheter actuator of FIG. 11;

FIG. 16A depicts a perspective view of another example of a body assembly that may be incorporated into a medical instrument, with an actuator in a proximal position;

FIG. 16B depicts a perspective view of the body assembly of FIG. 16A, with the actuator in a distal position;

FIG. 17 depicts an exploded perspective view of the body assembly of FIG. 16A;

FIG. 18 depicts a cross-sectional view of a handle of the body assembly of FIG. 16A, taken along line 18-18 of FIG. 16B;

FIG. 19 depicts a cross-sectional view of the handle of FIG. 18, taken along line 19-19 of FIG. 16B;

FIG. 20 depicts a perspective view of a guide rail hub body of a guide rail hub assembly of the body assembly of FIG. 16A;

FIG. 21 depicts a perspective view of a guide rail actuator of the guide rail hub assembly of the body assembly of FIG. 16A;

FIG. 22 depicts a front end view of the actuator of FIG. 16A;

FIG. 23 depicts a perspective view of the actuator of FIG. 16A;

FIG. 24 depicts a perspective view of an actuation assembly of the body assembly of FIG. 16A;

FIG. 25 depicts a perspective view of a first rack member of the actuation assembly of FIG. 24;

FIG. 26 depicts a perspective view of a second rack member of the actuation assembly of FIG. 24;

FIG. 27A depicts a cross-sectional side view of the body assembly of FIG. 16A, with the actuation assembly in a first state of operation;

FIG. 27B depicts a cross-sectional side view of the body assembly of FIG. 16A, with the actuation assembly in a second state of operation;

FIG. 27C depicts a cross-sectional side view of the body assembly of FIG. 16A, with the actuation assembly in a third state of operation;

FIG. 27D depicts a cross-sectional side view of the body assembly of FIG. 16A, with the actuation assembly in a fourth state of operation;

FIG. 27E depicts a cross-sectional side view of the body assembly of FIG. 16A, with the actuation assembly in a fifth state of operation;

FIG. 28A depicts a distal portion of a shaft assembly coupled with the body assembly of FIG. 16A, while the actuation assembly in the first state of operation of FIG. 27A;

FIG. 28B depicts a distal portion of a shaft assembly coupled with the body assembly of FIG. 16A, while the actuation assembly in the second state of operation of FIG. 27B;

FIG. 28C depicts a distal portion of a shaft assembly coupled with the body assembly of FIG. 16A, while the actuation assembly in the third state of operation of FIG. 27C;

FIG. 28D depicts a distal portion of a shaft assembly coupled with the body assembly of FIG. 16A, while the actuation assembly in the fourth state of operation of FIG. 27D;

FIG. 28E depicts a distal portion of a shaft assembly coupled with the body assembly of FIG. 16A, while the actuation assembly in the fifth state of operation of FIG. 27E;

FIG. 29 depicts a perspective view of another example of a body assembly that may be incorporated into a medical instrument, with a portion of a handle omitted to reveal internal components;

FIG. 30 depicts an enlarged, partial perspective view of components of an actuation assembly of the body assembly of FIG. 29, with a tab of a rack member of the actuation assembly spaced away from a notch of a sled member of the actuation assembly;

FIG. 31 depicts an enlarged, partial perspective view of the components of the actuation assembly of FIG. 30, with the tab disposed in the notch;

FIG. 32 depicts a perspective view of another example of a medical instrument, with a dilation catheter assembly in a proximal position;

FIG. 33 depicts a side elevation view of a distal portion of the instrument of FIG. 32, with an outer sheath omitted to reveal interior components of a shaft assembly;

FIG. 34 depicts a top plan view of the instrument components of FIG. 33;

FIG. 35 depicts a cross-sectional side view of the distal portion of the instrument of FIG. 32, taken along line 35-35 of FIG. 32;

FIG. 36 depicts an exploded perspective view of a dilation catheter assembly and guide rail assembly of the instrument of FIG. 32;

FIG. 37 depicts a perspective view of the guide rail assembly of FIG. 36;

FIG. 38 depicts a perspective view of the dilation catheter assembly of FIG. 36;

FIG. 39 depicts a perspective view of a manifold of the dilation catheter assembly of FIG. 36;

FIG. 40 depicts a cross-sectional perspective view of the manifold of FIG. 39, taken along line 40-40 of FIG. 39;

FIG. 41 depicts a cross-sectional perspective view of the dilation catheter assembly and guide rail assembly of FIG. 36, taken along line 35-35 of FIG. 32;

FIG. 42A depicts a perspective view of guide rail actuation components of the instrument of FIG. 32 in a first stage of operation;

FIG. 42B depicts a perspective view of the guide rail actuation components of FIG. 42B in a second stage of operation;

FIG. 42C depicts a perspective view of the guide rail actuation components of FIG. 42B in a third stage of operation;

FIG. 43A depicts a proximal end view of the dilation catheter assembly and guide rail assembly of FIG. 36, with the guide rail actuation components of the instrument of FIG. 32 in the first stage of operation of FIG. 42A;

FIG. 43B depicts a proximal end view of the dilation catheter assembly and guide rail assembly of FIG. 36, with the guide rail actuation components of the instrument of FIG. 32 in the second stage of operation of FIG. 42C;

FIG. 44 depicts a side elevation view of components of a shaft assembly of the instrument of FIG. 32, including reference planes indicating regions of varying malleability of a guide rail of the guide rail assembly of FIG. 36;

FIG. 45 depicts a perspective view of another example of a medical instrument, with a dilation catheter assembly in a proximal position;

FIG. 46A depicts a cross-sectional side view of a portion of the instrument of FIG. 45, in an example of a first state of operation;

FIG. 46B depicts a cross-sectional side view of a portion of the instrument of FIG. 45, in an example of a second state of operation;

FIG. 46C depicts a cross-sectional side view of a portion of the instrument of FIG. 45, in an example of a third state of operation;

FIG. 46D depicts a cross-sectional side view of a portion of the instrument of FIG. 45, in an example of a fourth state of operation;

FIG. 46E depicts a cross-sectional side view of a portion of the instrument of FIG. 45, in an example of a fifth state of operation;

FIG. 46F depicts a cross-sectional side view of a portion of the instrument of FIG. 45, in an example of a sixth state of operation;

FIG. 46G depicts a cross-sectional side view of a portion of the instrument of FIG. 45, in an example of a seventh state of operation;

FIG. 46H depicts a cross-sectional side view of a portion of the instrument of FIG. 45, in an example of an eighth state of operation;

FIG. 47 depicts an exploded perspective view of a portion of an actuation assembly of the instrument of FIG. 45;

FIG. 48 depicts an enlarged side view of a portion of the instrument of FIG. 45, with a portion of a handle omitted;

FIG. 49 depicts a perspective view of a guide rail actuation knob of the instrument of FIG. 45;

FIG. 50A depicts a cross-sectional end view of the instrument of FIG. 45, taken along line 50-50 of FIG. 48, with the guide rail actuation knob in a first angular position;

FIG. 50B depicts a cross-sectional end view of the instrument of FIG. 45, taken along line 50-50 of FIG. 48, with the guide rail actuation knob in a second angular position;

FIG. 50C depicts a cross-sectional end view of the instrument of FIG. 45, taken along line 50-50 of FIG. 48, with the guide rail actuation knob in a third angular position;

FIG. 51A depicts an enlarged side view of a portion of the instrument of FIG. 45, with a portion of the handle omitted, with the guide rail actuation knob in a proximal position, and with the guide rail actuation knob in the second angular position of FIG. 50B;

FIG. 51B depicts an enlarged side view of a portion of the instrument of FIG. 45, with a portion of the handle omitted, with the guide rail actuation knob in a distal position, and with the guide rail actuation knob in the second angular position of FIG. 50B;

FIG. 51C depicts an enlarged side view of a portion of the instrument of FIG. 45, with a portion of the handle omitted, with the guide rail actuation knob in the distal position, and with the guide rail actuation knob in the third angular position of FIG. 50C;

FIG. 51C depicts an enlarged side view of a portion of the instrument of FIG. 45, with a portion of the handle omitted, with the guide rail actuation knob in the distal position, and with the guide rail actuation knob in the third angular position of FIG. 50C;

FIG. 51D depicts an enlarged side view of a portion of the instrument of FIG. 45, with a portion of the handle omitted, with the guide rail actuation knob in the proximal position, and with the guide rail actuation knob in the third angular position of FIG. 50C;

FIG. 52 depicts a perspective view of another example of a rack member that may be incorporated into the instrument of FIG. 45, with a guide element disposed in a gripping element of the rack member;

FIG. 53 depicts an exploded perspective view of the rack member, guide element, and gripping element of FIG. 52;

FIG. 54 depicts a distal portion of another example of a guide element that may be incorporated into any of the instruments described herein; and

FIG. 55 depicts a distal portion of another example of a guide element that may be incorporated into any of the instruments described herein.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Examples of Dilation Instruments with Translatable Dilation Catheters

In some scenarios, it may be desirable to advance a dilation catheter into an anatomical passageway in or near the ear, nose, or throat of a patient; and expand the dilator to thereby dilate the passageway. For instance, it may be desirable to dilate a paranasal sinus ostium or other passageway associated with drainage of a paranasal sinus cavity, a Eustachian tube, a stenotic region in an airway of a patient, etc. It may also be desirable to incorporate a guide into such an instrument, to assist in guiding the dilation catheter into the targeted anatomical passageway; and to allow the dilation catheter to translate longitudinally relative to the guide. This may allow the guide to be initially positioned in relation to a targeted anatomical passageway while the dilation catheter is in a proximal position. The dilation catheter may then be advanced relative to the guide to a distal position to thereby enter the targeted anatomical passageway. The following provides several examples of how a dilation instrument may integrate an actuator to drive longitudinal movement of a dilation catheter.

A. Dilation Instrument with Rotary Dilation Catheter Actuator

As noted above, it may be desirable to have a dilation instrument that incorporates an actuator to drive translation of a dilation catheter relative to a guide. To the extent that some conventional instruments may provide a translating actuator to drive translation of a dilation catheter relative to a guide, some such translating actuators may provide usability difficulties. For instance, if an operator wishes to grasp the handle of the instrument with a single hand and actuate the actuator with the same hand (e.g., leaving their other hand free to manipulate an endoscope or other instrument, etc.), it may be difficult to advance the translating actuator through its full range of motion, particularly if the operator has small hands. It may therefore be desirable to provide a dilation catheter actuator that does not require the operator to move their finger or thumb through full range of longitudinal motion required to drive corresponding longitudinal motion of a dilation catheter.

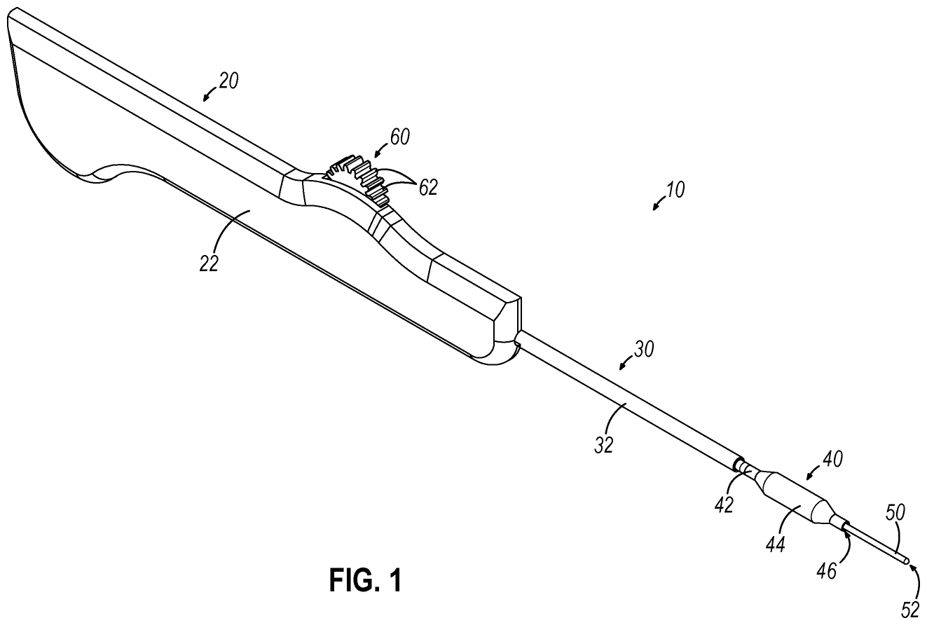

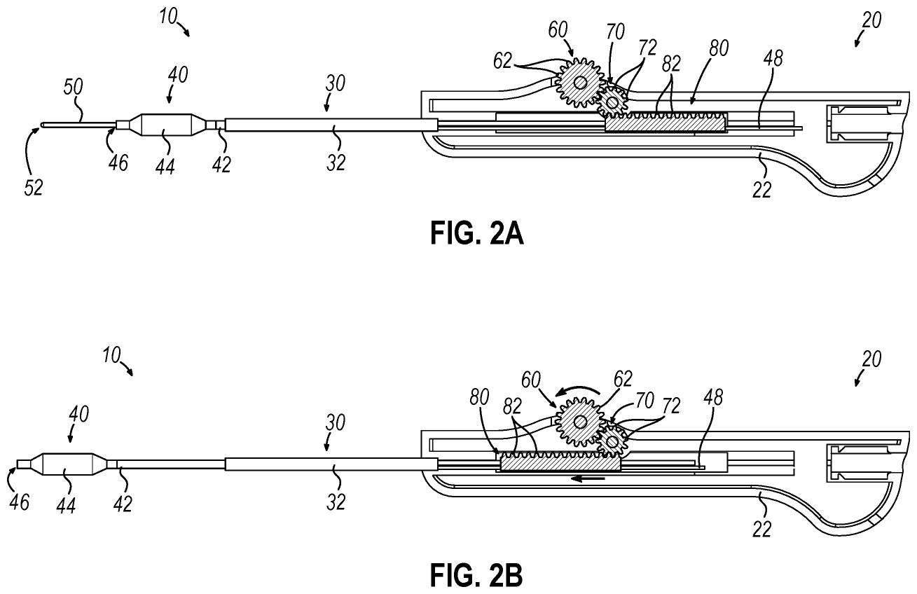

To that end, it may be desirable to provide a dilation instrument that provides a rotary actuator to drive longitudinal motion of a dilation catheter, to facilitate driving the dilation catheter through its full range of longitudinal motion without requiring the operator to move a finger or thumb through a corresponding range of longitudinal motion. FIGS. 1-2B depict an example of a dilation instrument (10) that provides longitudinal actuation of a dilation catheter (40) through rotary movement of an actuator (60). Dilation instrument (10) may be used to dilate an anatomical passageway in or near an ear, nose, or throat of a patient. Instrument (10) of this example includes a body assembly (12) and a shaft assembly (13). Body assembly (12) includes a first housing (22), a second housing (not shown, but may be positioned opposite to first housing (22)), and a dilation catheter actuator (60). Housings (22) cooperate to form a handle. Dilation catheter actuator (60) is rotatably supported by the handle. Dilation catheter actuator (60) is operable to drive longitudinal movement of a dilation catheter (40) as described in greater detail below.

Shaft assembly (30) includes an outer sheath (32), a guide rail (50), and dilation catheter (40). In the present example, these components are configured to be positioned coaxially with each other, such that outer sheath (32) is positioned externally, dilation catheter (40) is positioned internal to outer sheath (32), and guide rail (50) is positioned internal to dilation catheter (40). Outer sheath (32) of the present example is rigid, though other versions may be malleable or otherwise flexible. In the present example, outer sheath (32) does not enter the head of the patient during operation of instrument (10), though some scenarios may exist where outer sheath (32) enters the patient during operation of instrument (00).

Guide rail (50) of the present example is malleable and has an atraumatic distal tip (52). In some versions, distal tip (52) is dome shaped. In some other versions, distal tip (52) is enlarged (e.g., configured as a ball tip or blueberry tip, etc.). The malleability of guide rail (50) may allow guide rail (50) to maintain the bend angle of a bend while guide rail (50) is disposed in the head of the patient, including while dilation catheter (40) is advanced distally relative to guide rail (50). Such operability of guide rail (50) may promote access by dilation catheter (40) to various locations within the head of a patient, such as the maxillary sinus ostium, the frontal recess, the sphenoid sinus ostium, the Eustachian tube, etc., based on the selected bend angle. By way of example only, the bending of guide rail (50) may be performed in accordance with at least some of the teachings of U.S. Pat. No. 11,013,897, entitled “Apparatus for Bending Malleable Guide of Surgical Instrument,” issued May 25, 2021, the disclosure of which is incorporated by reference herein, in its entirety.

Dilation catheter (40) of the present example includes a shaft (42) having an integral balloon (44) and a distal tip (46). Shaft (42) is coaxially positioned with outer sheath (32) and guide rail (50). Shaft (42) defines two inner lumens, including a first lumen in which guide rail (50) is slidably disposed and a second lumen in fluid communication with balloon (44). Balloon (44) may comprise a non-extensible material and may be sized and configured to fit within a targeted anatomical passageway while in the deflated state; then dilate the targeted anatomical passageway while in the inflated state. It should be understood that balloon (44) is shown as being inflated in FIGS. 1A-2B for schematic purposes only; and that balloon (44) may in fact be deflated at such stages of operation.

One or both of distal tips (46, 52) may comprise an indicator element. Each such indicator element may comprise a position sensor and/or an illuminating feature. In versions where an indicator element includes a position sensor, the position sensor may comprise one or more coils. When such a coil is positioned within an alternating electromagnetic field generated by field generators, the alternating magnetic field may induce electrical current in the coil, and this induced electrical current may be communicated as a position-indicative signal along the electrical conduit(s) in the instrument and further to a processor. This phenomenon may enable an image-guided surgery navigation system to determine the location of distal tips (46, 52) within a three-dimensional space (i.e., within the head of the patient, etc.). To accomplish this, the processor may execute an algorithm to calculate location coordinates of distal tip (46, 52) from the position related signals (e.g., from induced currents) of the coil(s) in distal tip (46, 52). Thus, a navigation sensor may serve as a position sensor by generating signals indicating the real-time position of the sensor within three-dimensional space; or by otherwise indicating the real-time position of the sensor within three-dimensional space.

In versions where an indicator element includes an illuminating feature, the illuminating feature may be operable to project light outwardly from indicator element. Such an illuminating feature may provide transillumination through the skin of the patient. In some such versions, the illuminating feature includes an optically transmissive window that is optically coupled with one or more optical fibers, with such one or more optical fibers being optically coupled with a light source. In some other versions, the illuminating feature includes one or more LEDs or other local sources of light positioned locally at distal tip (46, 52).

FIGS. 2A-2B show components that are operable to drive translation of dilation catheter (40). As noted above, these components include a dilation catheter actuator (60) that is rotatably supported by the handle formed by housings (22). Dilation catheter actuator (60) of the present example is in the form of a pinion having teeth (62), where dilation catheter actuator (60) may be driven by a finger or thumb of the same hand that grasps the handle for one-handed operation of instrument (10). It should therefore be understood that, in some cases, dilation catheter actuator (60) may be operated as a thumbwheel. An idler gear (70) is also rotatably supported by housing (22) and has teeth (72) that mesh with teeth (62) of dilation catheter actuator (60), such that idler gear (70) rotates in response to rotation of dilation catheter actuator (60). A rack (80) is slidably supported by housing (22) and has teeth (82) that mesh with teeth (72) of idler gear (70), such that rack (80) translates in response to rotation of idler gear (70). Thus, when dilation catheter actuator (60) is rotated in a first direction, rack (80) translates distally from the proximal position shown in FIG. 2A to the distal position shown in FIG. 2B. When dilation catheter actuator (60) is rotated in a second direction, rack (80) translates proximally from the distal position shown in FIG. 2B to the proximal position shown in FIG. 2A.

A drive rod (48) is fixedly secured to rack (80), such that driver rod (48) translates unitarily with rack (80). A distal portion of drive rod (48) is fixedly coupled with shaft (42) of dilation catheter (40), such that dilation catheter (40) translates unitarily with drive rod (48). Thus, when dilation catheter actuator (60) is rotated in a first direction, dilation catheter (40) translates distally from the proximal position shown in FIG. 2A to the distal position shown in FIG. 2B. When dilation catheter actuator (60) is rotated in a second direction, dilation catheter (40) translates proximally from the distal position shown in FIG. 2B to the proximal position shown in FIG. 2A.

It should be understood from the foregoing that an operator may grasp the handle formed by housings (22) with a single hand, and readily manipulate dilation catheter actuator (60) with a single finger or thumb of that same hand to drive dilation catheter (40) distally and proximally along a full range of longitudinal motion. Moreover, the operator may achieve this full range of longitudinal motion of dilation catheter (40) without having to move the finger or thumb on dilation catheter actuator (60) along the same full range of longitudinal motion. In some cases, the operator may achieve the full range of longitudinal motion of dilation catheter (40) by urging dilation catheter actuator (60) to rotate via one stroke with the finger or thumb of the hand grasping the handle. In some other cases, the operator may drive dilation catheter actuator (60) with two or more strokes of the finger or thumb to drive dilation catheter (40) along the full range of longitudinal motion. In either scenario, this interaction with dilation catheter actuator (60) may be easier for some operators than manipulation of a dilation catheter actuator that must travel longitudinally along the same full range of longitudinal motion of dilation catheter (40), particularly operators with small hands.

While FIGS. 1-2B show dilation catheter (40) being advanced distally along a straight guide rail (50), dilation catheter (40) may also be advanced distally along a guide rail (50) having a bend formed therein, with guide rail (50) maintaining the formed bend as dilation catheter (40) traverses the formed bend to reach the targeted anatomical passageway. Regardless of whether guide rail (50) is bent or straight, after dilation catheter (40) reaches the distal position, balloon (44) may be inflated to dilate the targeted anatomical passageway.

While instrument (10) provides translation of dilation catheter (40) relative to an internal guide in the form of guide rail (50), some variations of instrument (10) may provide translation of dilation catheter relative to an external guide, such as a guide catheter or other kind of external guide. Such external guides may be rigid, malleable, or steerable. Versions of instrument (10) that include an external guide may omit guide rail (50). It should therefore be understood that the presence of guide rail (50) is not necessary for proper performance of dilation catheter actuator (60) in instrument (10).

B. Dilation Instrument with Elongate Sliding Dilation Catheter Actuator having Proximal Handle Entry

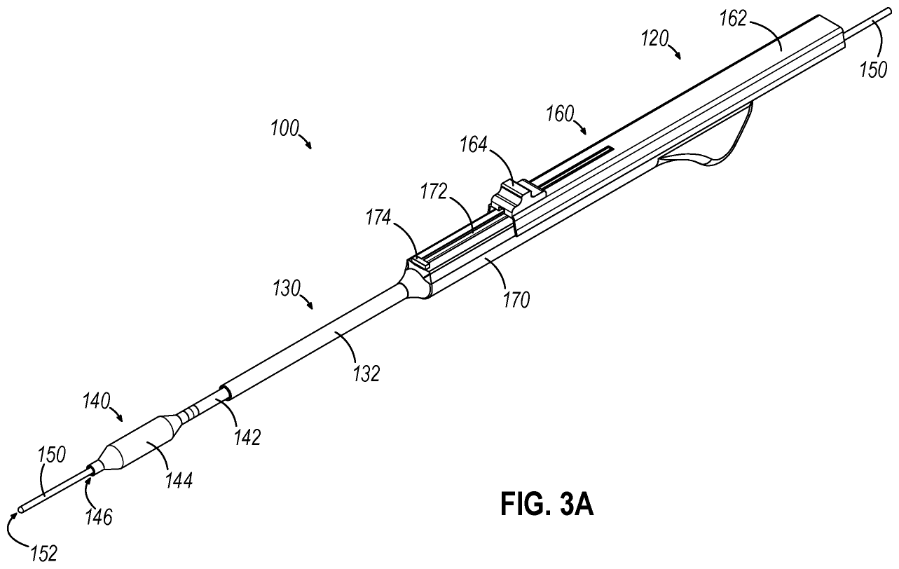

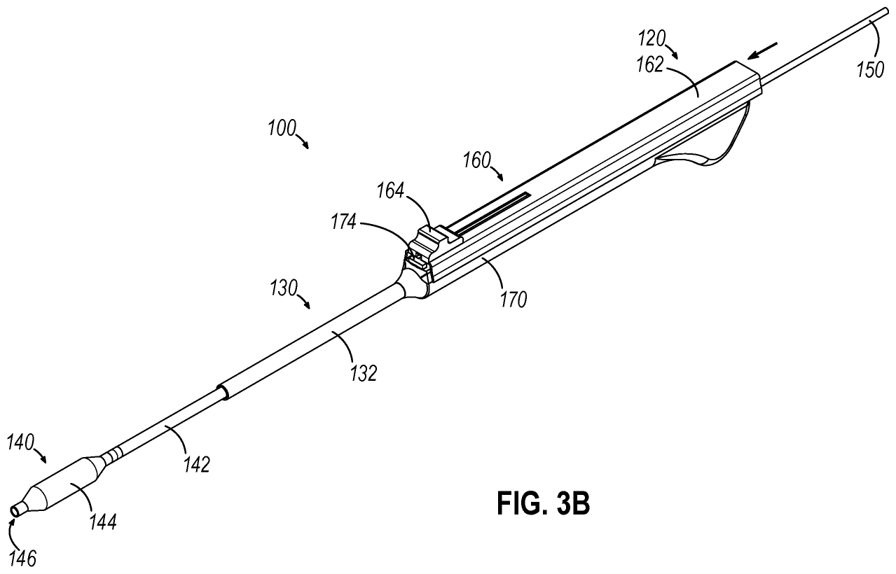

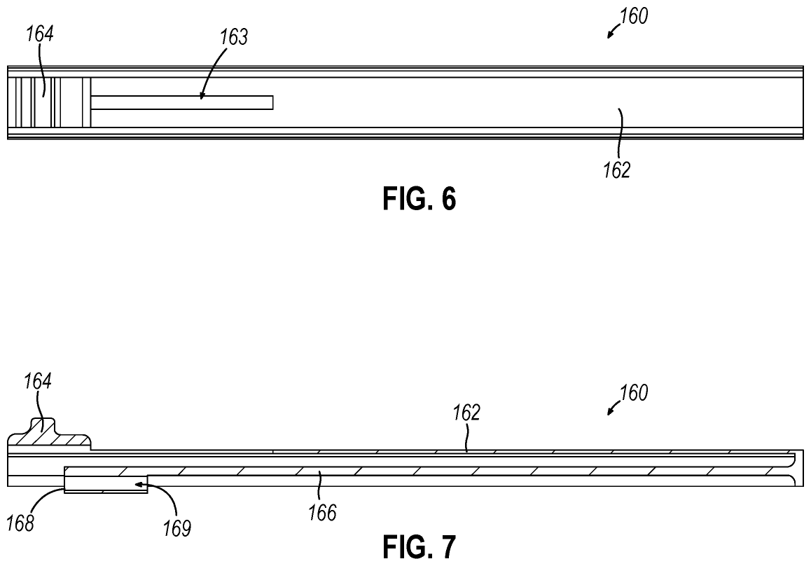

As noted above, it may be desirable to allow an operator to drive a dilation catheter through a full range of longitudinal motion without requiring the operator to drive a finger or thumb through the same range of longitudinal motion. It may also be desirable to allow an operator to readily actuate a translating dilation catheter actuator from different positions along the length of a handle of the dilation instrument, which may provide the operator with greater flexibility in where they may choose to grip the handle along the length of the dilation instrument. FIGS. 3A-10B show an example of a dilation instrument (100) having a dilation catheter actuator (160) that may be translated to drive translation of a dilation catheter (140) without necessarily requiring the operator to drive a finger or thumb through the same full range of longitudinal motion of dilation catheter. Moreover, as will be described in greater detail below, dilation catheter actuator (160) may be readily manipulated from different longitudinal positions along the length of a handle (170) of instrument (100), thereby providing substantial flexibility for different gripping positions that the operator may choose.

Dilation instrument (100) may be used to dilate an anatomical passageway in or near an ear, nose, or throat of a patient. Instrument (100) of this example includes a body assembly (120) and a shaft assembly (130). Body assembly (120) includes a handle body (170) and a dilation catheter actuator (160). Dilation catheter actuator (160) is slidably disposed along a rail (172) formed on an exterior of handle body (170). Dilation catheter actuator (160) is operable to drive longitudinal movement of a dilation catheter (140) as described in greater detail below.

Shaft assembly (130) includes an outer sheath (132), a guide rail (150), and dilation catheter (140). In the present example, these components are configured to be positioned coaxially with each other, such that outer sheath (132) is positioned externally, dilation catheter (140) is positioned internal to outer sheath (132), and guide rail (150) is positioned internal to dilation catheter (140). Outer sheath (132) of the present example is rigid, though other versions may be malleable or otherwise flexible. In the present example, outer sheath (132) does not enter the head of the patient during operation of instrument (100), though some scenarios may exist where outer sheath (132) enters the patient during operation of instrument (100).

Guide rail (150) of the present example is malleable and has an atraumatic distal tip (152). In some versions, distal tip (152) is dome shaped. In some other versions, distal tip (152) is enlarged (e.g., configured as a ball tip or blueberry tip, etc.). The malleability of guide rail (150) may allow guide rail (150) to maintain the bend angle of a bend while guide rail (150) is disposed in the head of the patient, including while dilation catheter (140) is advanced distally relative to guide rail (150). Such operability of guide rail (150) may promote access by dilation catheter (140) to various locations within the head of a patient, such as the maxillary sinus ostium, the frontal recess, the sphenoid sinus ostium, the Eustachian tube, etc., based on the selected bend angle. By way of example only, the bending of guide rail (150) may be performed in accordance with at least some of the teachings of U.S. Pat. No. 11,013,897, entitled “Apparatus for Bending Malleable Guide of Surgical Instrument,” issued May 25, 2021, the disclosure of which is incorporated by reference herein, in its entirety.

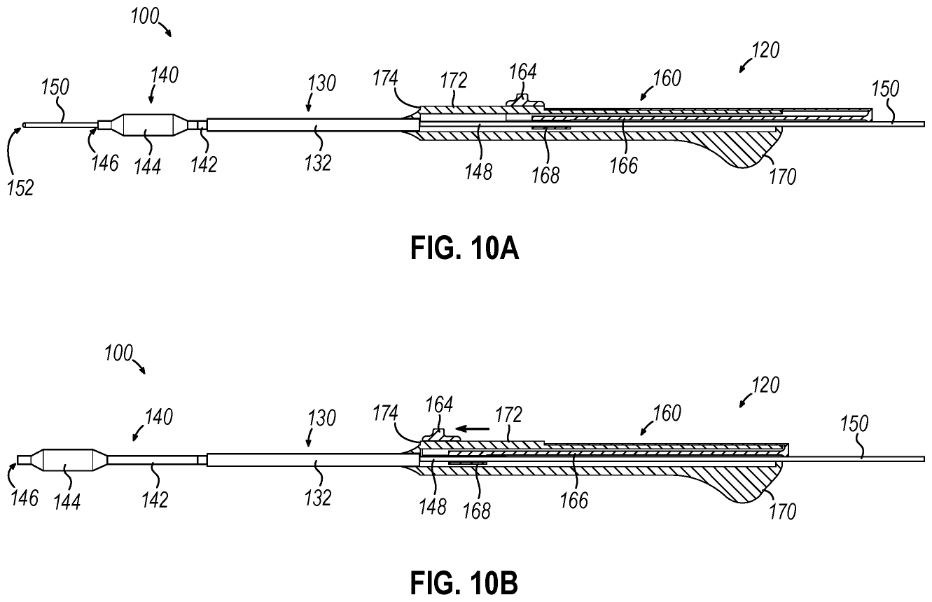

Dilation catheter (140) of the present example includes a shaft (142) having an integral balloon (144) and a distal tip (146). Shaft (142) is coaxially positioned with outer sheath (132) and guide rail (150). Shaft (142) defines two inner lumens, including a first lumen in which guide rail (150) is slidably disposed and a second lumen in fluid communication with balloon (144). Balloon (144) may comprise a non-extensible material and may be sized and configured to fit within a targeted anatomical passageway while in the deflated state; then dilate the targeted anatomical passageway while in the inflated state. It should be understood that balloon (144) is shown as being inflated in FIGS. 3A-3B and 10A-10B for schematic purposes only; and that balloon (144) may in fact be deflated at such stages of operation.

One or both of distal tips (146, 152) may comprise an indicator element. Each such indicator element may comprise a position sensor and/or an illuminating feature. In versions where an indicator element includes a position sensor, the position sensor may comprise one or more coils that provide signals in response to electromagnetic fields emitted by magnetic field generators of an image guided surgery system that is operable to determine the real-time position of the position sensor in three-dimensional space based on the signals provided by the position sensor. In versions where an indicator element includes an illuminating feature, the illuminating feature may be operable to project light outwardly from indicator element. Such an illuminating feature may provide transillumination through the skin of the patient. In some such versions, the illuminating feature includes an optically transmissive window that is optically coupled with one or more optical fibers, with such one or more optical fibers being optically coupled with a light source. In some other versions, the illuminating feature includes one or more LEDs or other local sources of light positioned locally at distal tip (146, 152).

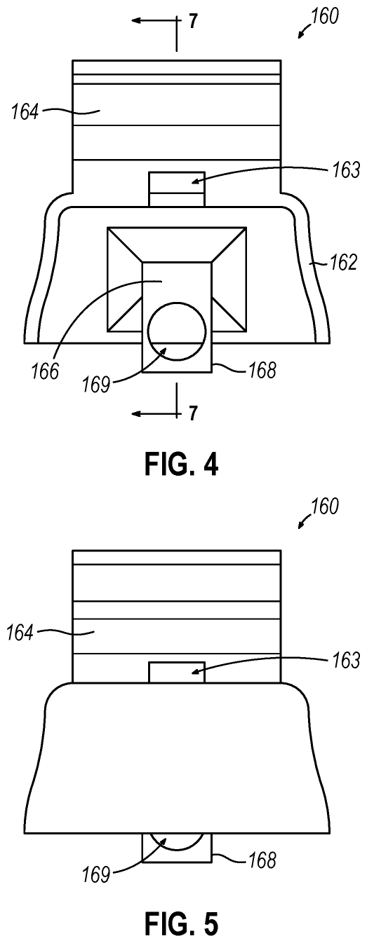

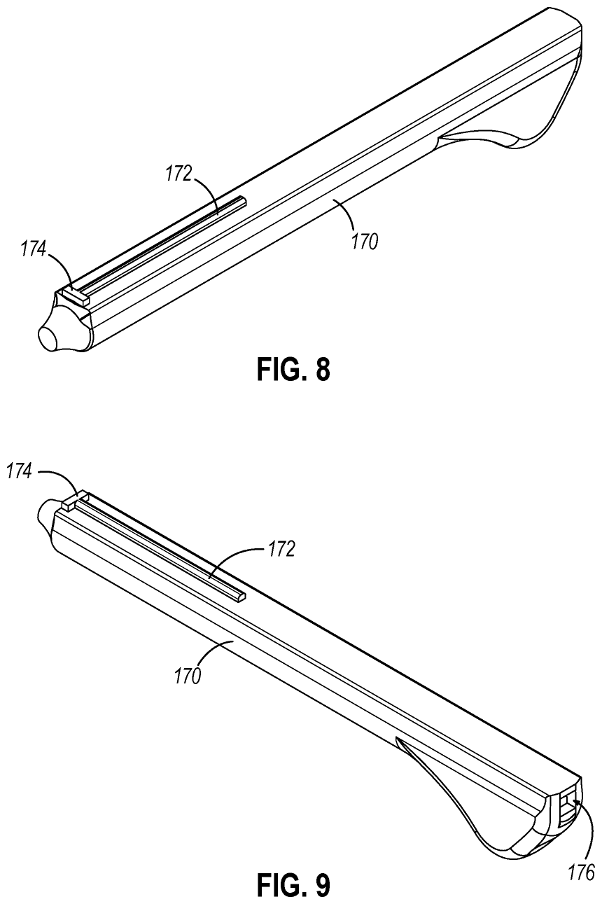

FIGS. 4-7 show dilation catheter actuator (160) in greater detail. As shown, dilation catheter actuator (160) of this example includes an elongate body (162) with an engagement ridge (164) at the distal end of body (162). An elongate slot (163) is formed in body (162) under engagement ridge (164) and proximal to engagement ridge (164). Slot (163) is configured to receive a rail (172) at the distal end of handle (170), which is best shown in FIGS. 8-9. Handle (170) thus slidably supports dilation catheter actuator (160) along the exterior of handle (170) through engagement between rail (172) and slot (163). A transversely oriented stop (174) is provided at the distal end of ridge (164) and is configured to engage the distal end of body (162) to thereby restrict distal advancement of dilation catheter actuator (160) along handle (170).

As best seen in FIGS. 4-5 and 7, an arm (166) protrudes distally from the proximal end of body. The distal end of arm (166) includes a block (168) defining an opening (169). Arm (166) is slidably disposed within an opening (176) that is formed at the proximal end of handle (170) and is best shown in FIG. 9. Arm (166) is freely disposed within a hollow interior region of handle (170), such that arm (166) is operable to translate between a proximal position as shown in FIG. 10A and a distal position as shown in FIG. 10B. A driver rod (148) is disposed in opening (169) and is fixedly secured to block (168). A distal portion of drive rod (148) is fixedly coupled with shaft (142) of dilation catheter (140), such that dilation catheter (140) translates unitarily with drive rod (148). Thus, when dilation catheter actuator (160) is translated distally along handle (170), dilation catheter (140) translates distally from the proximal position shown in FIG. 10A to the distal position shown in FIG. 10B. When dilation catheter actuator (160) is translated proximally along handle (170), dilation catheter (140) translates proximally from the distal position shown in FIG. 10B to the proximal position shown in FIG. 10A.

It should be understood from the foregoing that an operator may grasp handle (170) with a single hand, and readily manipulate dilation catheter actuator (160) with a single finger or thumb of that same hand to drive dilation catheter (140) distally and proximally along a full range of longitudinal motion. In some scenarios, the operator may achieve this full range of longitudinal motion of dilation catheter (140) by moving the finger or thumb on dilation catheter actuator (160) along the same full range of longitudinal motion, via one stroke with the finger or thumb of the hand grasping handle (170).

In some other cases, the operator may achieve the full range of longitudinal motion of dilation catheter (140) by urging dilation catheter actuator (160) longitudinally via two or more strokes of the finger or thumb to drive dilation catheter (140) along the full range of longitudinal motion. For instance, in a first stroke, the operator may urge dilation catheter (140) distally through a first range of longitudinal motion by pushing dilation catheter actuator (160) at engagement ridge (164). The operator may then release engagement ridge (164) and contact dilation catheter actuator (160) at a region proximal to engagement ridge (164), then urge dilation catheter (140) distally through a second range of longitudinal motion. This process may be repeated until dilation catheter (140) has achieved the full range of longitudinal motion.

It should be understood that this multi-stroke actuation process may be achieved due to the substantial length of dilation catheter actuator (160). By way of example only, the length of dilation catheter actuator (160) may be at least half the length of handle (170), or more particularly at least 75% of the length of handle (170), or more particularly substantially equal to the length of handle (170). It should also be understood that the substantial length of dilation catheter actuator (160) may allow the operator to grasp handle (170) at different longitudinal positions along the length of handle (170) and still readily manipulate dilation catheter actuator (160) at those various longitudinal positions along the length of handle (170). In some cases, the above-noted usability aspects provided by dilation catheter actuator (160) may be particularly beneficial to operators with small hands.

While FIGS. 3A-3B and 10A-10B show dilation catheter (140) being advanced distally along a straight guide rail (150), dilation catheter (140) may also be advanced distally along a guide rail (150) having a bend formed therein, with guide rail (150) maintaining the formed bend as dilation catheter (140) traverses the formed bend to reach the targeted anatomical passageway. Regardless of whether guide rail (150) is bent or straight, after dilation catheter (140) reaches the distal position, balloon (144) may be inflated to dilate the targeted anatomical passageway.

While instrument (100) provides translation of dilation catheter (140) relative to an internal guide in the form of guide rail (150), some variations of instrument (100) may provide translation of dilation catheter relative to an external guide, such as a guide catheter or other kind of external guide. Such external guides may be rigid, malleable, or steerable. Versions of instrument (100) that include an external guide may omit guide rail (150). It should therefore be understood that the presence of guide rail (150) is not necessary for proper performance of dilation catheter actuator (160) in instrument (100).

C. Elongate Sliding Dilation Catheter Actuator having Distal Handle Entry

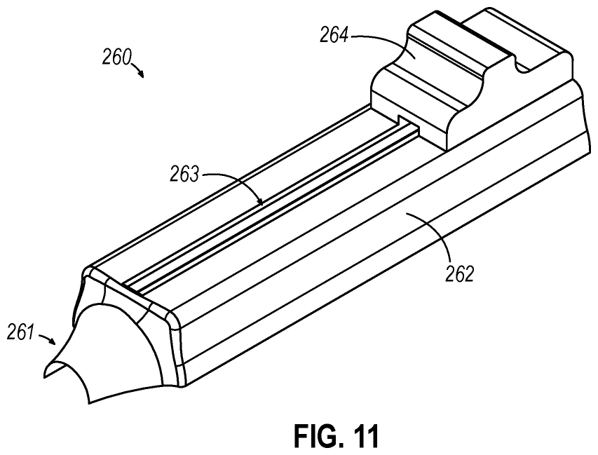

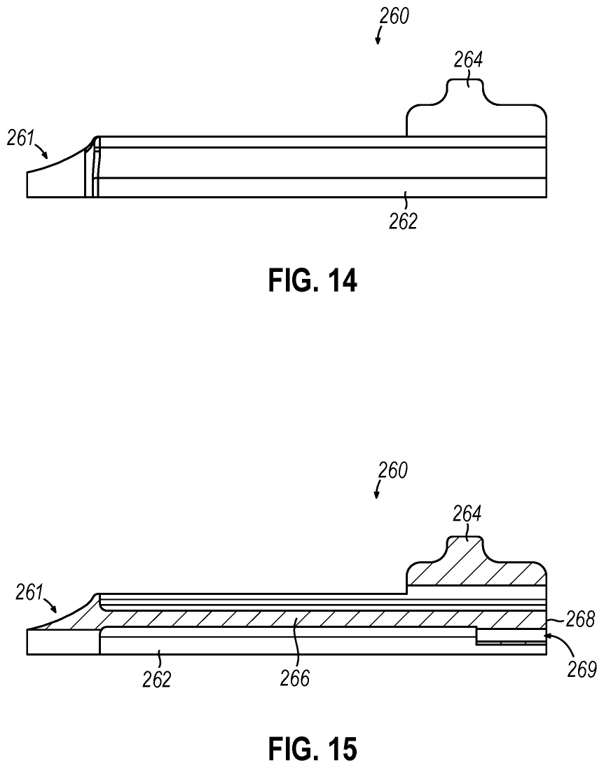

In some scenarios, it may be desirable to provide form and functionality similar to that of dilation catheter actuator (160), but without occupying space proximal to handle (170) when dilation catheter actuator (160) is in a proximal position. To that end, FIGS. 11-15 show an example of an alternative dilation catheter actuator (260) that may be used in place of dilation catheter actuator (160). It should be understood that dilation catheter actuator (260) of this example may be used with an alternative form of handle (170) where opening (176) is at the distal end of handle (170) rather than the proximal end of handle (170).

Dilation catheter actuator (260) of this example includes an elongate body (262) with an engagement ridge (264) at the proximal end of body (262) and a nose portion (261) at the distal end of body (262). An elongate slot (263) is formed in body (262) under engagement ridge (264) and distal to engagement ridge (264). Slot (263) is configured to receive a rail (e.g., like rail (172)) at the distal end of the (e.g., the form of handle (170) modified to include opening (176) at the distal end of handle (170)). The handle may thus slidably support dilation catheter actuator (260) along the exterior of the handle through engagement between the rail and slot (263).

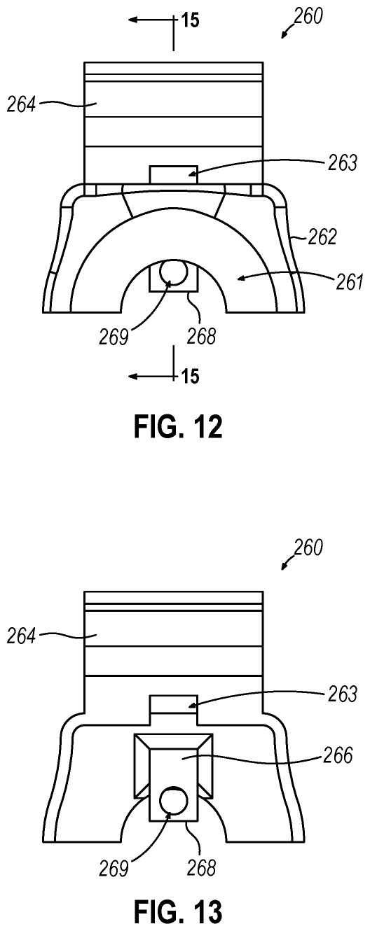

As best seen in FIGS. 12-13 and 15, an arm (266) protrudes proximally from the distal end of body. The proximal end of arm (266) includes a block (268) defining an opening (269). Arm (266) is slidably disposed within an opening that is formed at the distal end of the handle. Arm (266) is freely disposed within a hollow interior region of the handle, such that arm (266) is operable to translate between a proximal position and a distal position. Driver rod (148) may be disposed in opening (269) and be fixedly secured to block (268). Thus, when dilation catheter actuator (260) is translated distally along the handle, dilation catheter (140) translates distally from the proximal position to the distal position. When dilation catheter actuator (160) is translated proximally along handle (170), dilation catheter (140) translates proximally from the distal position to the proximal position.

Despite the differences in form between dilation catheter actuator (160) and dilation catheter actuator (260), dilation catheter actuator (260) may be operated in a manner similar to that described above in the context of dilation catheter actuator (160). Dilation catheter actuator (260) may also provide benefits similar to those described above in the context of dilation catheter actuator (160).

### II. Example of Dilation Instrument with Translatable Dilation Catheter, Adjustable Guide Rail, and Translating Guide Tip Element

As noted above, it may also be desirable to incorporate a guide into such an instrument, to assist in guiding the dilation catheter into the targeted anatomical passageway; and to allow the dilation catheter to translate longitudinally relative to the guide.

It may also be desirable to provide a single instrument that is capable of dilating different anatomical passageways in a patient. To facilitate such capabilities, the dilation instrument may include an adjustable guide rail, such as a malleable guide rail. To the extent that malleable properties may facilitate different bend angles in a guide rail, it may be further desirable to achieve different longitudinal positions and/or angular positions in a guide rail, to thereby further facilitate access to different anatomical passageways.

To the extent that an endoscope may be used to provide some degree of visualization within the ear, nose or throat of the patient, the nature of the anatomy may substantially restrict the field that is viewable by the endoscope. It may therefore be desirable to provide some other features to promote proper positioning of the dilator within the targeted anatomical passageway. For instance, the instrument may include a position sensor that provides signals indicating the location of the position sensor in three-dimensional space, such that an image-guided navigation system may be utilized to provide the operator with feedback indicating the real-time position of one or more features of the instrument in three-dimensional space. In addition, or in the alternative, the instrument may include an illuminating feature that is operable to transmit light through the skin of the patient (e.g., from within a frontal sinus cavity or maxillary sinus cavity, etc.); such that the transillumination may also provide feedback to the operator indicating where part of the medical instrument is positioned in real time.

In some scenarios, the distal end of a dilation catheter is positioned proximally relative to the distal end of a guide feature as the guide feature is initially advanced to a point near the anatomical passageway targeted for dilation. After the distal end of the guide feature reaches a desired position, the dilation catheter may be advanced distally relative to the guide feature, such that the distal end of the dilation catheter enters the targeted anatomical passageway and is positioned distally relative to the distal end of the guide feature. In such scenarios, it may be desirable to first obtain feedback indicating the real time position of the distal end of the guide feature in three-dimensional space while the dilation catheter is in the proximal position; then obtain feedback indicating the real time position of the distal end of the dilation catheter in three-dimensional space while the dilation catheter is in the distal position. To the extent that it may be possible to include a first navigation element (e.g., first position sensor or first illumination source, etc.) at the distal tip of the guide element and a second navigation element (e.g., second position sensor or first illumination source, etc.) at the distal tip of the dilation catheter, it may be desirable to avoid including two navigation elements to thereby reduce cost and complexity of the instrument.

The following provides examples of arrangements where a single dilation instrument may (1) integrate an actuator to drive longitudinal movement of a dilation catheter; (2) provide a guide rail having a combination of adjustable bend angles and adjustable angular positions; and (3) utilize a single navigation element to provide feedback indicating the real time position of the distal end of a guide feature in three-dimensional space while a dilation catheter is in the proximal position; then obtain feedback indicating the real time position of the distal end of the dilation catheter in three-dimensional space while the dilation catheter is in the distal position.

A. Body Assembly with Dual Rack Actuation Assembly

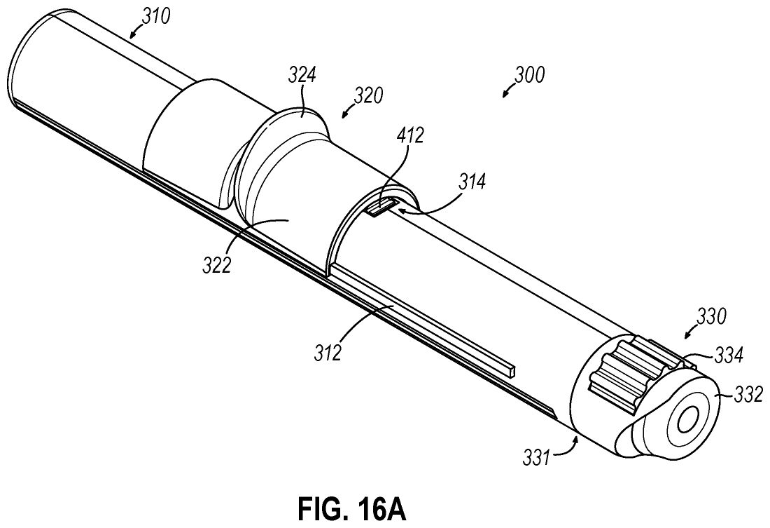

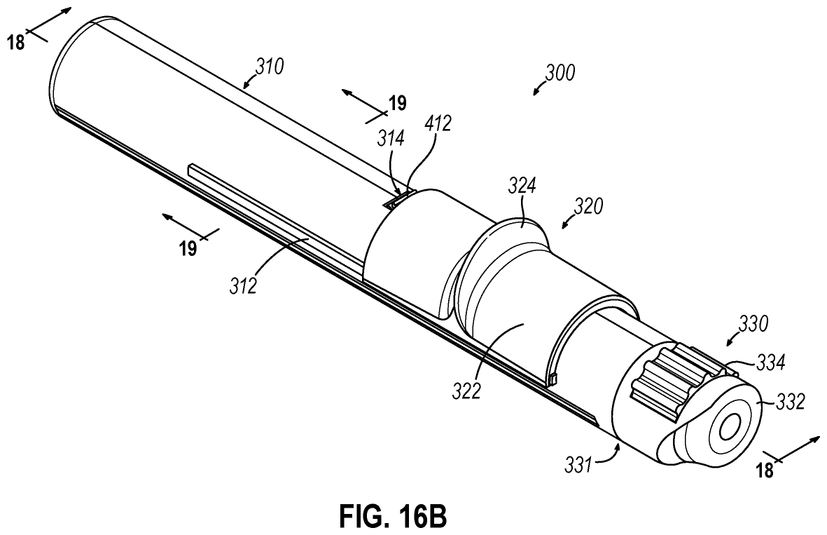

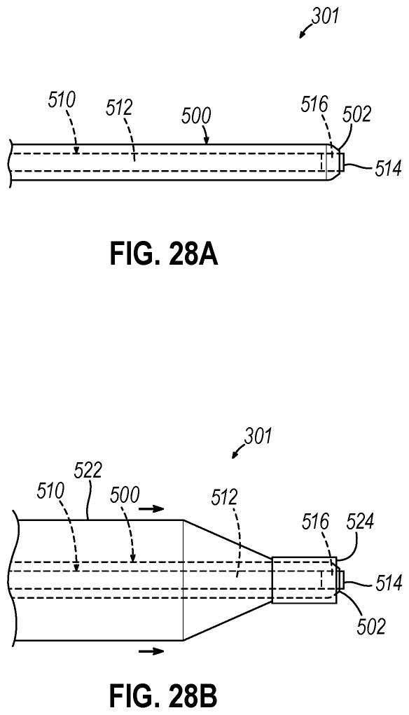

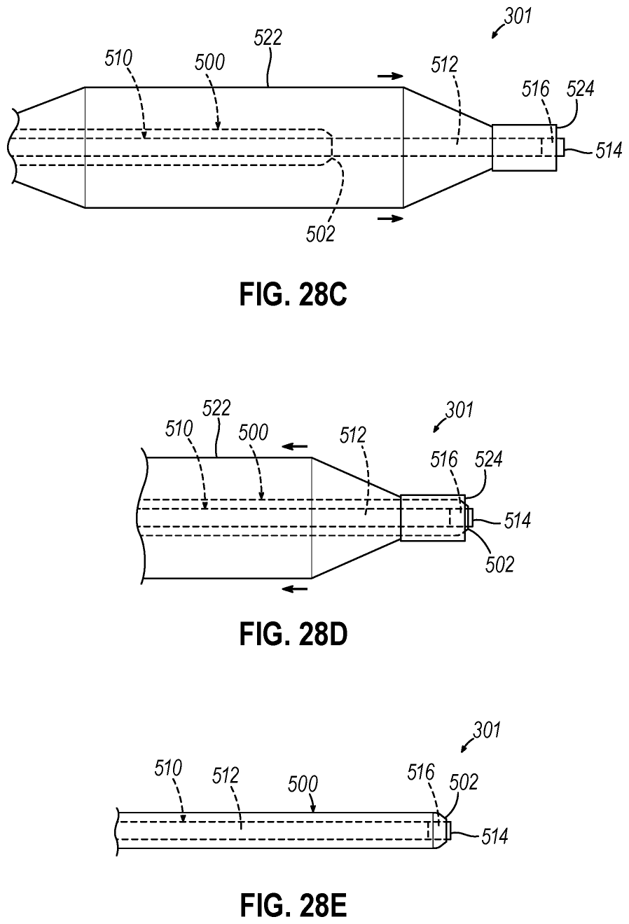

FIGS. 16A-16B show an example of a body assembly (300) that may be integrated into a dilation instrument such as any of the dilation instruments (10, 100) described herein. As described in greater detail below with reference to FIGS. 28A-28E, a shaft assembly (301) that includes a guide rail (500), a dilation catheter (520), and a guide element (510) may be coupled with body assembly (300) and extend distally relative to body assembly (300). Body assembly (300) of this example includes a handle (310), an actuator (320), and a guide rail hub assembly (330). Handle (310) is configured to be manipulated and grasped by the hand of an operator. Actuator (320) is slidably coupled with handle (310) and is operable to translate relative to handle (310) between a proximal position (FIG. 16A) and a distal position (FIG. 16B). Guide rail hub assembly (300) is coupled with handle (310) and is operable to drive rotation of guide rail (500) relative to handle (310) as described in greater detail below.

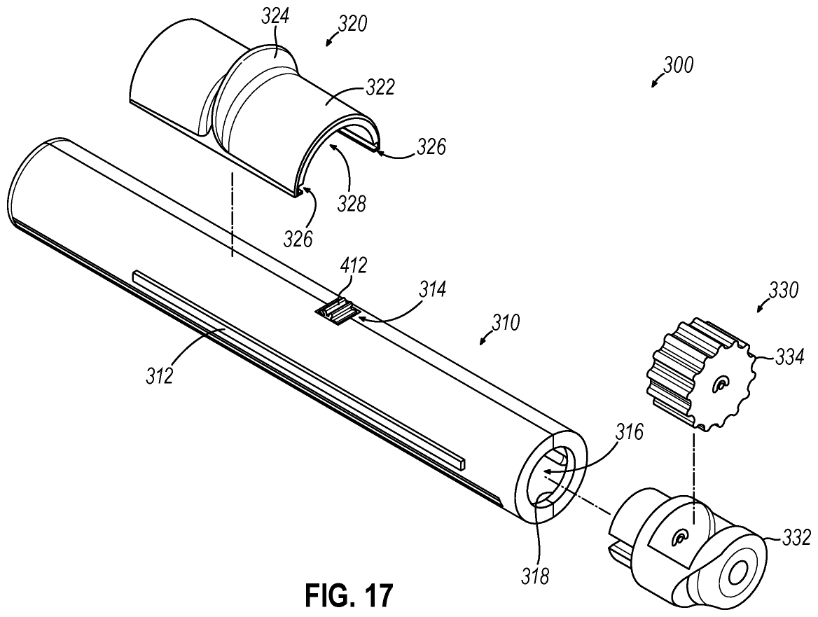

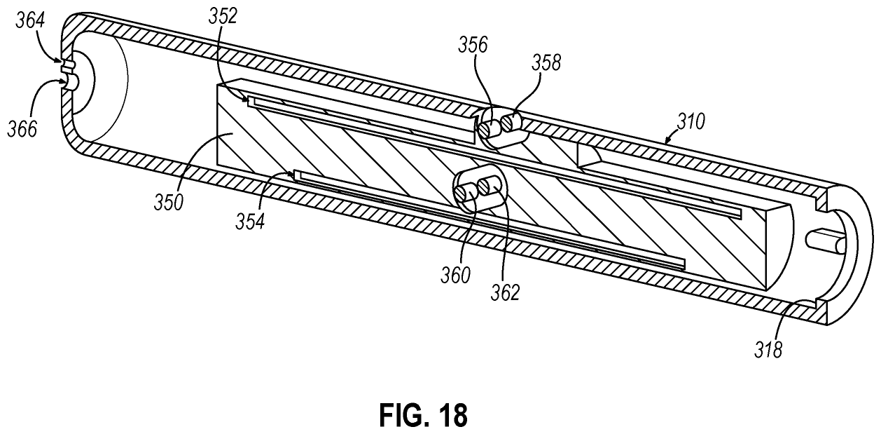

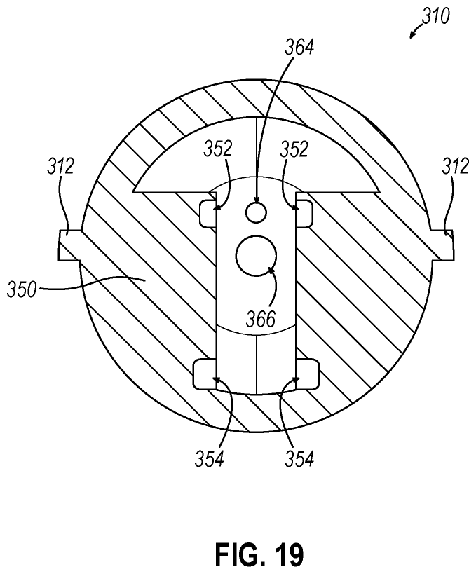

As shown in FIGS. 16A-17 and 19, handle (310) of the present example includes a pair of side rails (312) that extend laterally and longitudinally. As described below, side rails (312) are configured to slidably support actuator (320). As shown in FIGS. 16A-17, handle (310) defines an upper opening (314), through which a portion of a pinion (412) protrudes as also described below. As shown in FIG. 17, handle (310) further defines a distal opening (316) bounded by an inwardly extending annular flange (318). Annular flange (318) is configured to support guide rail hub assembly (300) as also described below. As shown in FIGS. 18-19, the interior of handle (310) includes a boss structure (350) that defines a pair of upper channels (352) and a pair of lower channels (354). Channels (352, 354) extend longitudinally along boss structure (350). As also shown in FIGS. 18-19, a first opening (364) and a second opening (366) are formed at the proximal end of handle (310). As shown in FIG. 18, a set of upper axles (356, 358) and a set of lower axles (360, 362) are also located within handle (310). In some versions, axles (356, 358, 360, 362) are integrally formed (e.g., molded, etc.) in handle (310). In some other versions axles (356, 358, 360, 362) comprise separately-formed pins that are secured within handle (310). Alternatively, axles (356, 358, 360, 362) may be formed in any other suitable configuration.

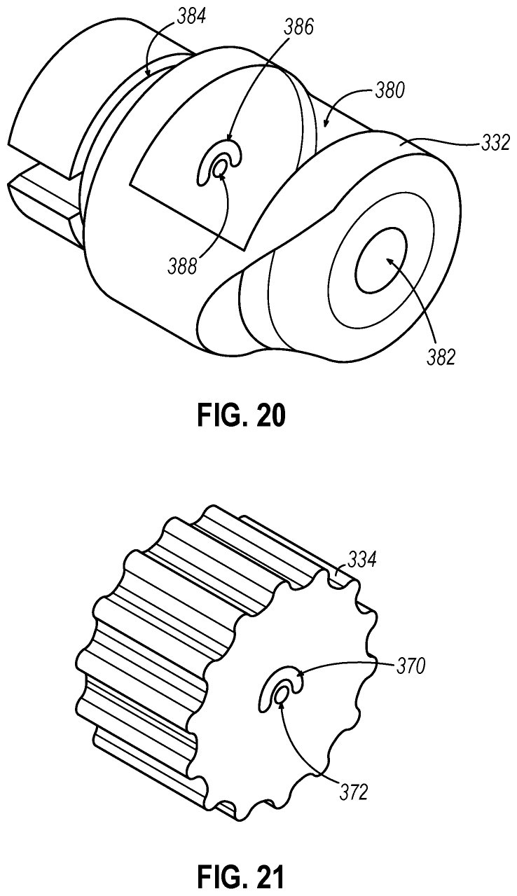

As best seen in FIG. 17, guide rail hub assembly (300) of the present example comprises a guide rail hub body (332) and a guide rail hub actuator (334). As best seen in FIG. 20, guide rail hub body (332) includes a laterally presented recess (380), a distal opening (382), a proximal annular recess (384), a first channel (386), and a second channel (388). Laterally presented recess (380) is configured to receive guide rail hub actuator (334) and allow guide rail hub actuator (334) to rotate relative to guide rail hub body (332), leaving a portion of guide rail hub actuator (334) exposed for contact with a thumb or other finger of an operator. Distal opening (382) is sized to receive the above-noted components of shaft assembly (301) shown in FIGS. 28A-28E. In some versions, shaft assembly (301) also includes an additional outer tubular support member (not shown). Such an outer tubular support member may be fixedly secured relative to guide rail hub body (332); and may allow components such as guide rail (500), dilation catheter (520), a guide element (510) to move relative to the outer tubular support member. In some such versions, the outer tubular support member may restrict lateral deflection of the proximal region of shaft assembly (301) relative to body assembly (300) and/or serve any other suitable purpose(s).

Proximal annular recess (384) is configured to receive annular flange (318) of handle (310), such that handle (310) and guide rail hub body (332) are coupled together through cooperation between annular flange (318) and annular recess (384). In some versions, this coupling allows guide rail hub body (332) to rotate relative to handle (310). In addition, or in the alternative, some versions may allow guide rail hub body (332) to translate relative to handle (310) to some degree. It should therefore be understood that guide rail hub body (332) need not necessarily be rigidly secured relative to handle (310), though some variations may in fact provide rigid securement between guide rail hub body (332) and handle (310). In some versions where guide rail hub body (332) is rotatable relative to handle (310), guide rail hub actuator (334) may be omitted. In some other versions where guide rail hub body (332) is rotatable relative to handle (310), guide rail hub actuator (334) may be coupled with guide rail hub body (332) in a manner such that guide rail hub actuator (334) is not rotatable relative to guide rail hub body (332).

First channel (386) of the present example extends along an arc; and is configured to communicate with at least a portion of a similarly arcuate channel (370) of guide rail hub actuator (334), which is best shown in FIG. 21. In the present example, channels (386, 370) are configured to slidably receive a proximal portion of dilation catheter (520). In particular, channels (386, 370) are configured to allow dilation catheter (520) to translate longitudinally relative to guide rail hub assembly (330), despite a portion of dilation catheter (520) being disposed in channels (386, 370). Moreover, the arcuate configurations of channels (386, 370) are configured to allow guide rail hub actuator (334) to rotate relative to guide rail hub body (332) without providing interference with respect to the portion of dilation catheter (520) disposed in channels (386, 370). In versions where guide rail hub body (332) rotates relative to handle (310), the arcuate configuration of first channel (386) may similarly allow guide rail hub body (332) to rotate relative to handle (310) without providing interference with respect to the portion of dilation catheter (520) disposed in channel (386).

As described in greater detail below and shown in FIGS. 28A-28E, dilation catheter (520) is coaxially positioned relative to guide rail (500) and guide element (510) at the distal region of shaft assembly (301). As also described in greater detail below, guide rail (500) and guide element (510) are coaxially positioned with channels (372, 388), which are laterally offset from channels (386, 370). It should therefore be understood that there may be a manifold hub or other component (not shown) that is positioned distal to guide rail hub actuator (334) that couples a first portion of dilation catheter (520) (i.e., the portion of dilation catheter (520) that extends through channels (386, 370)) with a second portion of dilation catheter (520) (i.e., the portion of dilation catheter (520) that is distal to guide rail hub actuator (334). This manifold hub or other component may provide mechanical communication of longitudinal translation motion from the first portion of dilation catheter (520) to the second portion of dilation catheter (520); and may also provide fluid communication of inflation fluid from the first portion of dilation catheter (520) to the second portion of dilation catheter (520).

Second channel (388) is configured to align with a corresponding channel (372) of guide rail hub actuator (334), which is best shown in FIG. 21. In the present example, channels (372, 388) are coaxial with the axis about which guide rail (500) rotates. In some versions, the proximal end of guide rail (500) is fixedly secured within channel (372), such that guide rail (500) does not enter channel (388); and such that guide rail (500) rotates unitarily with guide rail hub actuator (334) relative to guide rail hub body (332). In some other versions, particularly versions where guide rail hub body (332) is rotatable relative to handle (310), the proximal end of guide rail (500) may enter channel (388) and be fixedly secured therein, such that guide rail (500) rotates unitarily with guide rail hub body (332) relative to handle (310). In either scenario, the proximal end of guide rail (500) may be positioned distally relative to the distal end of handle (310). In other words, there may be various versions where guide rail (500) never enters handle (310), such that handle (310) is not positioned along any portion of guide rail (500), and such that no portion of guide rail (500) is positioned within handle (310). It should also be understood that, in the various examples described above, guide rail (500) does not extend from handle (310), that guide rail (500) is not fixed to handle (310), that guide rail (500) is not secured to handle (310), and that guide rail (500) is not sealed to handle (310).

In versions where the proximal end of guide rail (500) is only positioned in channel (372) of guide rail hub actuator (334), and guide rail (500) does not enter channel (388), guide element (510) may nevertheless be slidably disposed in channel (388). In such versions, guide element (510) may slidably pass through channel (388) and slidably enter the proximal end of guide rail (500) in channel (372) as described below.

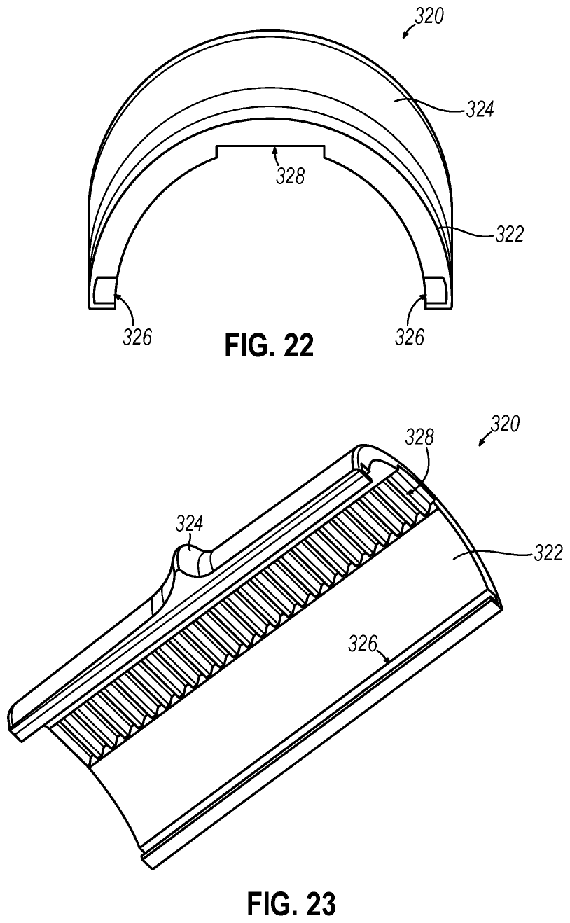

FIGS. 22-23 show actuator (320) in further detail. As shown, actuator (320) includes an arcuate body (322) with a ridge (324) that is configured to facilitate pushing and pulling engagement with a thumb or other finger of a hand of an operator. Body (322) further defines a pair of longitudinally extending recesses (326) that are configured to slidably receive side rails (312) of handle (310). The underside of actuator (320) includes an integral rack (328). The teeth of rack (328) are configured to mesh with the teeth of pinion (412) that are exposed through upper opening (314) handle (310) as described in greater detail below.

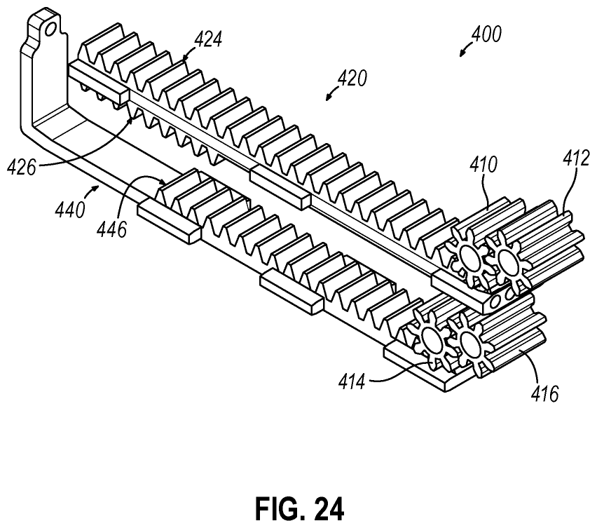

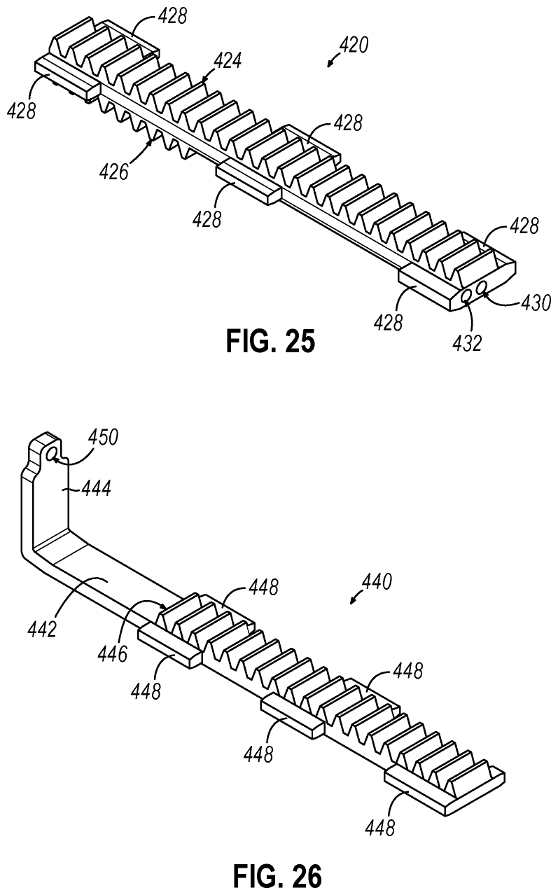

FIG. 24 shows an actuation assembly (400) that is incorporated into handle (310). Actuation assembly (400) of this example includes a first set of pinions (410, 412), a second set of pinions (414, 416), a first rack member (420), and a second rack member (440). As best seen in FIG. 25, first rack member (420) includes an elongate body (422) having an integral upper rack (424) and an integral lower rack (426). A set of rail segments (428) extend outwardly from body (422) and are configured to slidably fit in upper channels (352) of boss structure (350) in handle (310), such that boss structure (350) slidably supports first rack member (420). Body (422) further defines a first passageway (430) and a second passageway (432). In the present example, a proximal portion of dilation catheter (520) is fixedly secured within second passageway (432), such that dilation catheter (520) translates unitarily with first rack member (420) relative to handle (310). An inflation conduit of dilation catheter (520) is slidably disposed in opening (366) of handle (310), such that the inflation conduit of dilation catheter (520) slides freely through opening (366) when dilation catheter (520) translates unitarily with first rack member (420) relative to handle (310).

Also in the present example, guide element (510) is slidably disposed in first passageway (430). Guide element (510) is not fixed in first passageway (430), such that guide element (510) may translate relative to first rack member (420) during a portion of the range of motion when first rack member (420) translates relative to handle (310) as described in greater detail below. After exiting the distal end of first passageway (430), guide element continues to extend distally into the proximal end of guide rail (500). As noted above, the proximal end of guide rail (500) is located in guide rail hub assembly (330), at a longitudinal position that is distal to the distal end of handle (310).

As best seen in FIG. 26, second rack member (440) includes an elongate body (442) having an integral upper rack (446) and a proximal upright portion (444). A set of rail segments (448) extend outwardly from body (442) and are configured to slidably fit in lower channels (354) of boss structure (350) in handle (310), such that boss structure (350) slidably supports second rack member (440). Proximal upright portion (444) defines a passageway (450). A portion of guide element (510) is fixedly secured within passageway (450), such that guide element (510) translates unitarily with second rack member (440) relative to handle (310). Guide element (510) is further slidably disposed in opening (364) of handle (310), such that guide element (510) slides freely through opening (364) when guide element (510) translates unitarily with second rack member (440) relative to handle (310).

As noted above, handle (310) includes axles (356, 358, 360, 362). Pinion (410) of actuation assembly (400) is rotatably mounted to axle (356), pinion (412) is rotatably mounted to axle (358), pinion (414) is rotatably mounted to axle (360), and pinion (416) is rotatably mounted to axle (362). As also noted above, a portion of pinion (412) protrudes through opening (314) of handle (310) for meshing engagement with rack (328) of actuator (320). Thus, as actuator (320) is translated along handle (310) between the proximal position (FIG. 16A) and a distal position (FIG. 16B), this meshing engagement between rack (328) and pinion (412) causes pinion (412) to rotate about axle (356). Pinion (412) is also in meshing engagement with pinion (410), such that rotation of pinion (412) about axle (356) causes pinion (410) to rotate about axle (354). Pinion (410) is also in meshing engagement with upper rack (424) of first rack member (420), such that rotation of pinion (410) causes first rack member (420) to translate longitudinally. Due to these meshing relationships between racks (328, 424) and pinions (410, 412), distal advancement of actuator (320) along handle (310) provides distal translation of first rack member (420) within handle (310); and proximal advancement of actuator (320) along handle (310) provides proximal translation of first rack member (420) within handle (310).

During a certain stage of operation of actuation assembly (400) as described in greater detail below, lower rack (426) of first rack member (420) eventually achieves meshing engagement with pinion (416), such that translation of first rack member (420) drives rotation of pinion (416) about axle (362). Pinion (416) is also in meshing engagement with pinion (414), such that rotation of pinion (416) about axle (362) causes pinion (414) to rotate about axle (354). Pinion (414) is also in meshing engagement with rack (446) of second rack member (440), such that rotation of pinion (414) causes second rack member (440) to translate longitudinally.

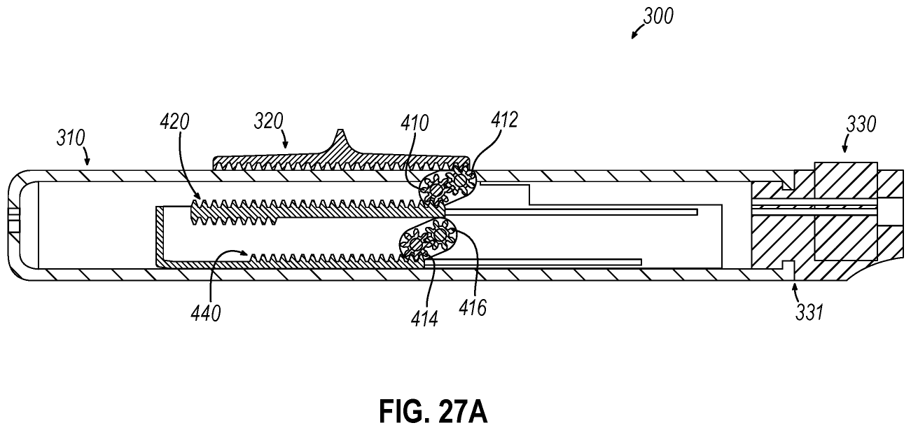

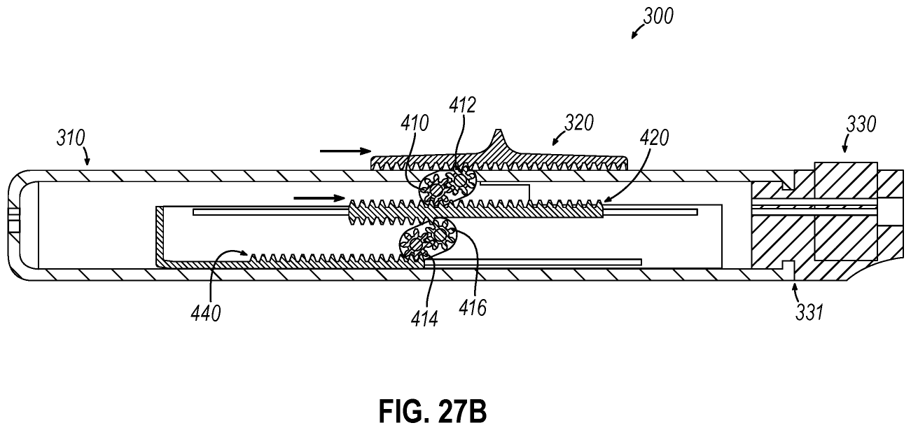

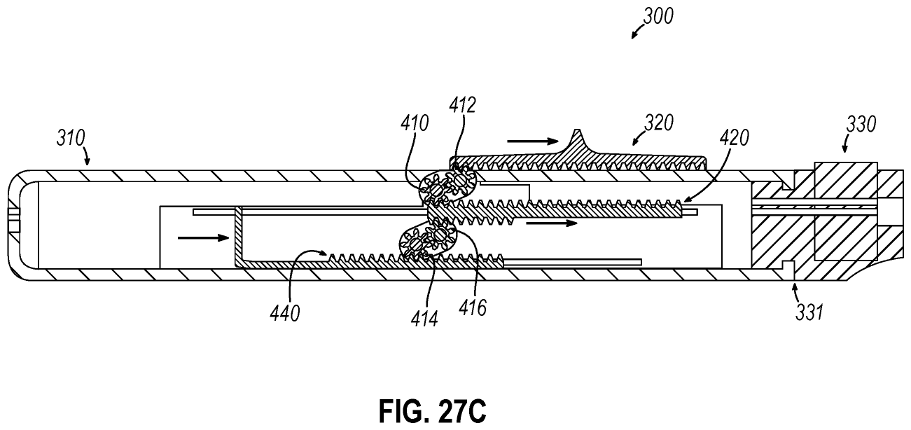

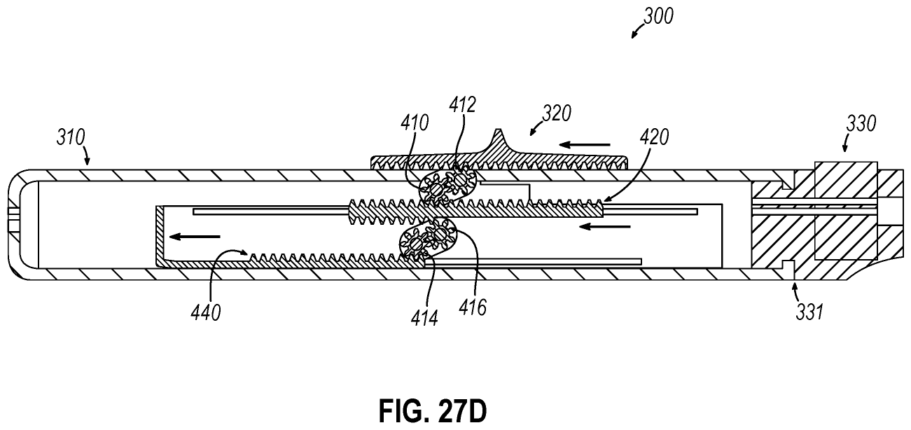

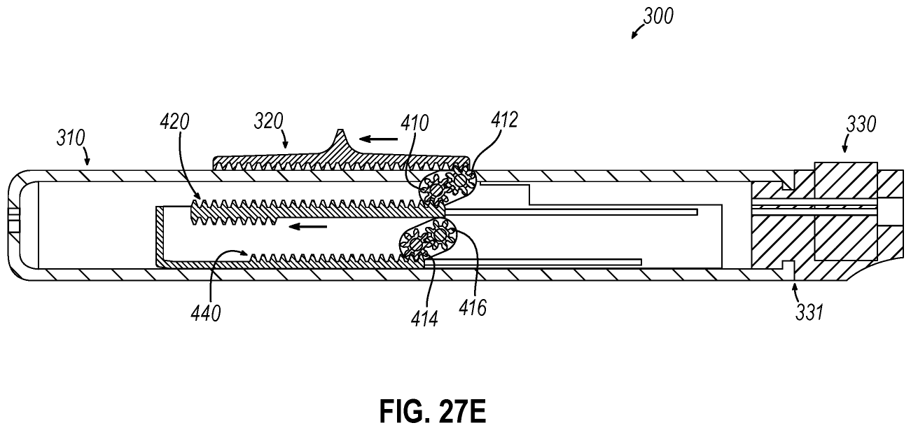

FIGS. 27A-27E show actuation assembly (400) at various stages of operation based on the longitudinal position of actuator (320) along handle (310). FIGS. 28A-28E shows the components of shaft assembly (301) at the same stages of operation.

As shown in FIGS. 28A-28E, the components of shaft assembly (301) are configured to be positioned coaxially with each other, such that guide rail (500) is positioned internal to dilation catheter (520), and guide element (510) is positioned internal to guide rail (500). Guide rail (500) of the present example is malleable and has an atraumatic distal tip (502). In some versions, distal tip (502) is dome shaped. In some other versions, distal tip (502) is enlarged (e.g., configured as a ball tip or blueberry tip, etc.). The malleability of guide rail (500) allows guide rail (500) to be bent to a desired bend angle before being inserted into the head of the patient. The malleability of guide rail (500) may allow guide rail (500) to maintain the bend angle while guide rail (500) is disposed in the head of the patient, including while dilation catheter (520) is advanced distally relative to guide rail (500). Such operability of guide rail (500) may promote access by dilation catheter (520) to various locations within the head (H) of a patient (P), such as the maxillary sinus ostium, the frontal recess, the sphenoid sinus ostium, the Eustachian tube, etc., based on the selected bend angle. By way of example only, the bending of guide rail (500) may be performed in accordance with at least some of the teachings of U.S. Pat. No. 11,013,897, entitled “Apparatus for Bending Malleable Guide of Surgical Instrument,” issued May 25, 2021, the disclosure of which is incorporated by reference herein, in its entirety. Guide rail (500) defines an inner lumen, in which guide element (510) is slidably disposed.

Guide element (510) of the present example comprises a shaft (512) having a distal tip (514). By way of example only, shaft (512) may comprise a polymeric material, a metallic material, a tubular structure, a helical structure (e.g., similar to a guidewire, etc.), and/or any other suitable material(s) or configuration(s). An indicator element (516) is positioned at distal tip (514) of guide element (510). Indicator element (516) is configured to indicate the position of distal tip (514) in three-dimensional space.

In some versions, indicator element (516) comprises one or more position sensors. For instance, indicator element (516) may comprise one or more coils that provide signals in response to electromagnetic fields emitted by magnetic field generators of an image-guided surgery system, and a processor of the image-guided surgery system is operable to determine the real-time position of distal tip (514) in three-dimensional space based on the signals provided by indicator element (516). In such versions, one or more wires, conductive traces, or other electrically conductive elements may extend from indicator element (516) along at least part of the length of guide element (510). Signals from indicator element (516) may be communicated to the processor via wire or wirelessly.

In addition to comprising a position sensor, or in lieu of comprising a position sensor, indicator element (516) may comprise an illuminating feature that is operable to project light outwardly from indicator element (516). Such an illuminating feature may provide transillumination through the skin of the patient as described above. In some such versions, indicator element (516) includes an optically transmissive window that is optically coupled with one or more optical fibers, with such one or more optical fibers extending along at least part of the length of guide element (510) to a light source. In some such versions, the proximal end of guide element (510) includes a connector (e.g., luer feature, etc.) that allows the operator to optically couple guide element (510) with various kinds of light sources, such as light sources providing different colors of light. In some other versions, indicator element (516) includes one or more LEDs or other local sources of light. In some such versions, one or more wires, conductive traces, or other electrically conductive elements may extend from indicator element (516) along at least part of the length of guide element (510) to a source of electrical power. It should be understood that indicator element (516) may comprise a position sensor, an illuminating feature, or a combination of a position sensor and an illuminating feature.

FIGS. 27A and 28A show body assembly (300) and shaft assembly (301) and in an initial stage of operation, where dilation catheter (520) is in a proximal position. As shown in FIG. 27A, actuator (320) and first rack member (420) are proximally positioned relative to handle (310) at this stage. As shown in FIG. 28A, distal tip (514) of guide element (510) is positioned at distal tip (502) of guide rail (500). Also in this state, distal end distal tip (524) of dilation catheter (520) is spaced proximally from distal tips (502, 514). Thus, a distal region of guide rail (500) is exposed relative to dilation catheter (520). While guide rail (500) is shown in a straight configuration in FIG. 28A, guide rail (500) may alternatively be in a bent configuration as noted above. The operator may select and execute the desired bend angle in guide rail (500) based on the location and/or configuration of the targeted anatomical passageway. Shaft assembly (301) may be inserted into the patient (P) while dilation catheter (520) is in the proximal position shown in FIG. 28A. With distal tip (514) of guide element (510) positioned at distal tip (502) of guide rail (500), the real-time position feedback provided by indicator element (516) may effectively indicate the real-time position of distal tip (502) in three-dimensional space.

Once the operator has sufficiently positioned distal tip (502) at the desired location, based on feedback from indicator element (516), the operator may begin advancing actuator (320) distally along handle (310) as shown in FIG. 27B. This distal advancement of actuator (320) drives first rack member (420) distally as shown in FIG. 27B. The distal advancement of first rack member (420) drives dilation catheter (520) distally as shown in FIG. 28B. During the distal range of travel of dilation catheter (520), distal tip (524) eventually reaches the same longitudinal position of distal tips (502, 514), as shown in FIG. 28B. Up to this point of operation, second rack member (440) and guide element (510) remain stationary, as lower rack (426) has not yet started to drive rotation of pinion (416); though lower rack (426) has just made initial contact with pinion (416).

As the operator continues to advance actuator (320) distally along handle (310) as shown in FIG. 27C, this further advancement of actuator (320) drives first rack member (420) further distally as shown in FIG. 27C. Moreover, since lower rack (426) is now in meshing engagement with pinion (416), the further advancement of actuator (320) drives second rack member (440) distally with first rack member (420). Thus, as shown in FIG. 28C, as dilation catheter (520) continues to advance distally, guide element (510) translates distally with dilation catheter (520), maintaining distal tip (514) of guide element (510) at the same longitudinal position as distal tip (524) of dilation catheter (520). Thus, with distal tip (514) of guide element (510) positioned at distal tip (524) of dilation catheter (520), the real-time position feedback provided by indicator element (516) may effectively indicate the real-time position of distal tip (524) in three-dimensional space.

After reaching the distal position shown in FIGS. 27C and 28C, the operator may inflate balloon (522) to dilate the targeted anatomical passageway. In some cases, balloon (522) may be inflated and deflated repeatedly to achieve sufficient dilation. While balloon (522) is shown in the inflated state in FIGS. 28B-28D, it should be understood that balloon (522) may in fact be in a deflated state during the stages of operation shown in FIGS. 28B and 28D. Similarly, balloon (522) may in fact be in a deflated state during part(s) of the stage of operation shown in FIG. 28C.

When the dilation is complete, balloon (522) may be deflated and retracted proximally as shown in the transition from FIGS. 27C and 28C to FIGS. 27D and 28D. As shown, dilation catheter (520) eventually reaches the position where distal tip (524) reaches the same longitudinal position of distal tips (502, 514). At this point, lower rack (426) disengages pinion (416) as shown in FIG. 27E, such that guide element (510) ceases further proximal movement while dilation catheter (520) continues to translate proximally; and such that distal tip (514) of guide element (510) remains at distal tip (502) of guide rail (500) as shown in FIG. 28E.

In view of the foregoing, during the stage of operation between that shown in FIGS. 27A and 28A and that shown in FIGS. 27B and 28B, the real-time position feedback provided by indicator element (516) may effectively indicate the real-time position of distal tip (502) in three-dimensional space. During the stage of operation between that shown in FIGS. 27B and 28B and that shown in FIGS. 27C and 28C, the real-time position feedback provided by indicator element (516) may effectively indicate the real-time position of distal tip (524) in three-dimensional space. During the stage of operation between that shown in FIGS. 27C and 28C and that shown in FIGS. 27D and 28D, the real-time position feedback provided by indicator element (516) may continue to effectively indicate the real-time position of distal tip (524) in three-dimensional space. During the stage of operation between that shown in FIGS. 27D and 28D and that shown in FIGS. 27E and 28E, the real-time position feedback provided by indicator element (516) may again effectively indicate the real-time position of distal tip (502) in three-dimensional space.

The foregoing teachings may be readily combined with at least some of the teachings of U.S. Provisional Pat. App. No. 63/467,679, entitled “Guide Rail Actuation Assembly for Balloon Dilation Instrument,” filed May 19, 2023, the disclosure of which is incorporated by reference herein, in its entirety. By way of example only, the aspects described above with respect to guide rail (500) and/or other aspects of shaft assembly (301) may be provided in accordance with at least some of the teachings of U.S. Provisional Pat. App. No. 63/467,679. In addition, or in the alternative, the foregoing teachings may be readily combined with at least some of the teachings of U.S. Provisional Pat. App. No. 63/467,683, entitled “Balloon Dilation Instrument with Translating Guide Tip Element,” filed May 19, 2023, the disclosure of which is incorporated by reference herein, in its entirety. By way of example only, the aspects described above with respect to guide element (510) and/or other aspects of shaft assembly (301) may be provided in accordance with at least some of the teachings of U.S. Provisional Pat. App. No. 63/467,683.

B. Body Assembly with Rack and Sled Actuation Assembly

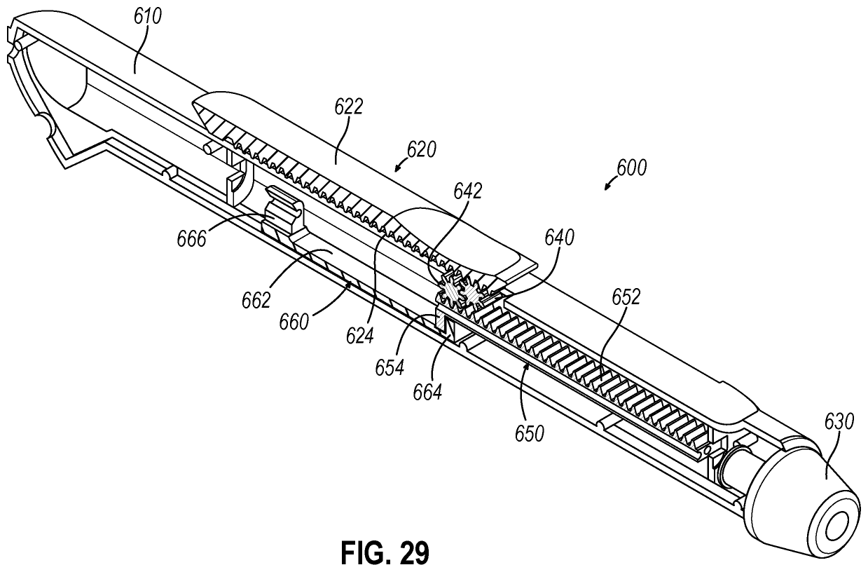

FIG. 29 shows an example of another body assembly (600) that may be integrated into a dilation instrument such as any of the dilation instruments (10, 100) described herein. It should be understood that shaft assembly (301) described above with reference to FIGS. 28A-28E may be readily coupled with body assembly (600) and extend distally relative to body assembly (600). Body assembly (600) of this example includes a handle (610), an actuator (620), and a guide rail hub assembly (630). Handle (610) is configured to be manipulated and grasped by the hand of an operator. Actuator (620) is slidably coupled with handle (610) and is operable to translate relative to handle (610) between a proximal position and a distal position. In particular, handle (610) includes a pair of side rails (not shown) that extend laterally and longitudinally. Such side rails of handle (610) may be similar to side rails (312) of handle (310), such that the side rails of handle (610) are configured to slidably support actuator (620). As shown in FIG. 29, handle (610) also defines an upper opening, through which a portion of a pinion (640) protrudes as also described below.

Guide rail hub assembly (630) is coupled with handle (610) and is operable to drive rotation of guide rail (500) relative to handle (610) as described above. By way of example only, guide rail hub assembly (630) may be configured and operable like guide rail hub assembly (330) or in any other suitable fashion. The proximal end of guide rail (500) may be positioned distally relative to the distal end of handle (610). In other words, there may be various versions where guide rail (500) never enters handle (610), such that handle (610) is not positioned along any portion of guide rail (500), and such that no portion of guide rail (500) is positioned within handle (610). It should also be understood that, in the various examples described above, guide rail (500) does not extend from handle (610), that guide rail (500) is not fixed to handle (610), that guide rail (500) is not secured to handle (610), and that guide rail (500) is not sealed to handle (610).

Body assembly (600) further includes an actuation assembly that, like actuation assembly (400) of body assembly (300), is operable to drive staged longitudinal translation of guide element (510) and dilation catheter (520). The actuation assembly of body assembly (600) includes a pair of pinions (640, 642), a rack member (650), and a sled member (660). Pinions (640, 642) are rotatably supported by corresponding axles, which are secured to handle (610) similar to axles (356, 358, 360, 362) described above. A portion of pinion (640) protrudes through an opening of handle (610), similar to the protrusion of pinion (412) through opening (314) of handle (310) as described above. The protruding portion of pinion (412) is in meshing engagement with teeth of a rack (624) that is integrally formed on the underside of actuator (620). The exterior (622) of actuator (620) is configured to be engaged by a thumb or other finger of the hand of the operator, such that the operator may engage exterior (622) to drive translational movement of actuator (620) along handle (610). This translational movement of actuator (620) along handle (610) causes rotation of pinion (640), due to the meshing engagement between pinion (640) and the teeth of rack (624). Pinion (640) also meshes with pinion (642), such that rotation of pinion (640) causes rotation of pinion (642). Translation of actuator (620) along handle (610) thus causes rotation of both pinions (640, 642).

Rack member (650) and sled member (660) are each slidably supported within handle (610) via corresponding channels, which are similar to channels (352, 354) described above. Rack member (650) includes an upper rack (652), which meshes with pinion (642) such that rotation of pinion (642) drives translation of rack (652) along handle (610). Dilation catheter (520) is fixedly secured to rack member (650), such that dilation catheter (520) translates unitarily with rack member (650) relative to handle (610). An inflation conduit of dilation catheter (520) is slidably disposed in a proximal opening of handle (610), such that the inflation conduit of dilation catheter (520) slides freely through the proximal opening of handle (610)) when dilation catheter (520) translates unitarily with rack member (650) relative to handle (610).

Also in the present example, guide element (510) is slidably supported disposed in a passageway formed through rack member (650). Guide element (510) is not fixed in the corresponding passageway of rack member (650), such that guide element (510) may translate relative to rack member (650) during a portion of the range of motion when rack member (650) translates relative to handle (610). After exiting the distal end of rack member (650), guide element continues to extend distally into the proximal end of guide rail (500). As noted above, the proximal end of guide rail (500) is located in guide rail hub assembly (630), at a longitudinal position that is distal to the distal end of handle (610).

Sled member (660) includes an upper cam surface (662), a distal upright portion (664), and a proximal upright portion (666). A portion of guide element (510) is fixedly secured to proximal upright portion (666), such that guide element (510) translates unitarily with sled member (660) relative to handle (610). Guide element (510) is further slidably disposed in a proximal opening of handle (610), such that guide element (510) slides freely through the proximal opening of handle (610) when guide element (510) translates unitarily with sled member (660) relative to handle (610).

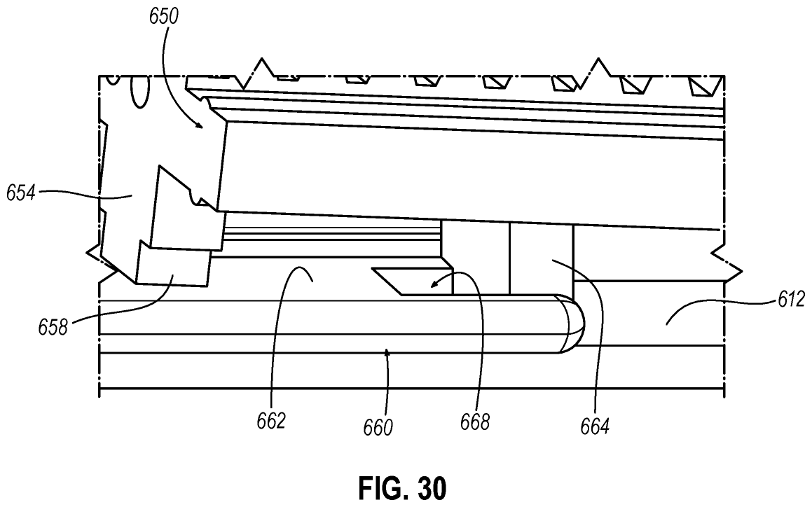

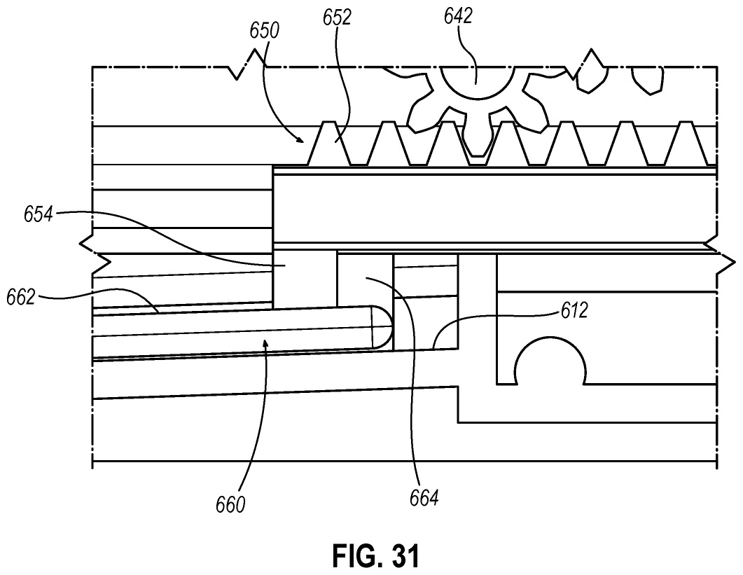

Sled member (660) is slidably supported along an obliquely oriented bearing surface (612) defined within handle (610). This provides at least a distal portion of sled member (660) at an orientation that is oblique relative to rack member (650), such that the distal end of sled member (660) is positioned closer to a horizontal plane passing through rack member (650) than the proximal end of sled member (660). This arrangement provides selective coupling between rack member (650) and sled member (660), based on the longitudinal position of rack member (650). In particular, and as best seen in FIG. 30, a tab (658) of a downward projection (654) at the proximal end of rack member (650) is configured to travel over cam surface (662) of sled member (660) as rack member (650) travels from a proximal position (similar to what is shown in FIG. 27A) to an intermediate position (similar to what is shown in FIG. 27B). During this range of travel of rack member (650) from the proximal position to the intermediate position, distal movement of rack member (650) does not drive sled member (660) distally. In addition, during this range of travel of rack member (650) from the proximal position to the intermediate position, dilation catheter (520) advances distally while guide element (510) remains longitudinally stationary, as shown in the transition from FIG. 28A to FIG. 28B.

When rack member (650) reaches the intermediate position, tab (658) reaches a notch (668) formed at the distal end of sled member (660), just proximal to distal upright portion (664). When tab (658) reaches notch (668), tab (658) enters notch (668). As rack member (650) continues to advance distally from the intermediate position (similar to what is shown in FIG. 27B) to a distal position (similar to what is shown in FIG. 27C), downward projection (654) of rack member (650) bears against distal upright portion (664) of sled member (660), such that sled member (660) translates distally with rack member (650). This joint distal translation of rack member (650) and sled member (660) provides joint distal translation of dilation catheter (520) and guide element (510), similar to what is shown in the transition from FIG. 28B to FIG. 28C.

After actuator (620) reaches the distal position and balloon (522) has been expanded as desired to provide dilation and/or some other effect(s), the operator may retract actuator (620) back toward the proximal position. This may cause proximal retraction of rack member (650) proximally, due to meshing engagement between racks (624, 652) and pinions (640, 642) as described above. As rack member (650) retracts proximally, rack member (650) may drive sled member (660) proximally due to engagement of tab (658) in notch (668). This joint proximal translation of rack member (650) and sled member (660) provides joint proximal translation of dilation catheter (520) and guide element (510), similar to what is shown in the transition from FIG. 28C to FIG. 28D. When rack member (650) reaches the intermediate position during proximal translation, sled member (660) reaches a point along bearing surface (612) where enough vertical separation is created between the distal end of sled member (660) and the proximal end of rack member (650), such that tab (658) exits notch (668). At this point, rack member (650) no longer drives rack member (650) proximally, such that guide element (510) remains stationary while dilation catheter (520) continues to translate proximally as shown in the transition from FIG. 28D to FIG. 28E.

### III. Examples of Dilation Instrument with Adjustable Guide Rail

In some scenarios, it may be desirable to advance a dilation catheter into an anatomical passageway in or near the ear, nose, or throat of a patient; and expand the dilator to thereby dilate the passageway. For instance, it may be desirable to dilate a paranasal sinus ostium or other passageway associated with drainage of a paranasal sinus cavity, a Eustachian tube, a stenotic region in an airway of a patient, etc. It may also be desirable to provide a single instrument that is capable of dilating different anatomical passageways in a patient. To facilitate such capabilities, the dilation instrument may include an adjustable guide, such as a malleable guide. To the extent that a malleable guide may facilitate different bend angles in a guide, it may be further desirable to achieve different longitudinal positions and/or angular positions in a guide, to thereby further facilitate access to different anatomical passageways. The following describes examples of dilation instruments with a guide having a combination of adjustable bend angles, adjustable longitudinal positions, and/or adjustable angular positions.

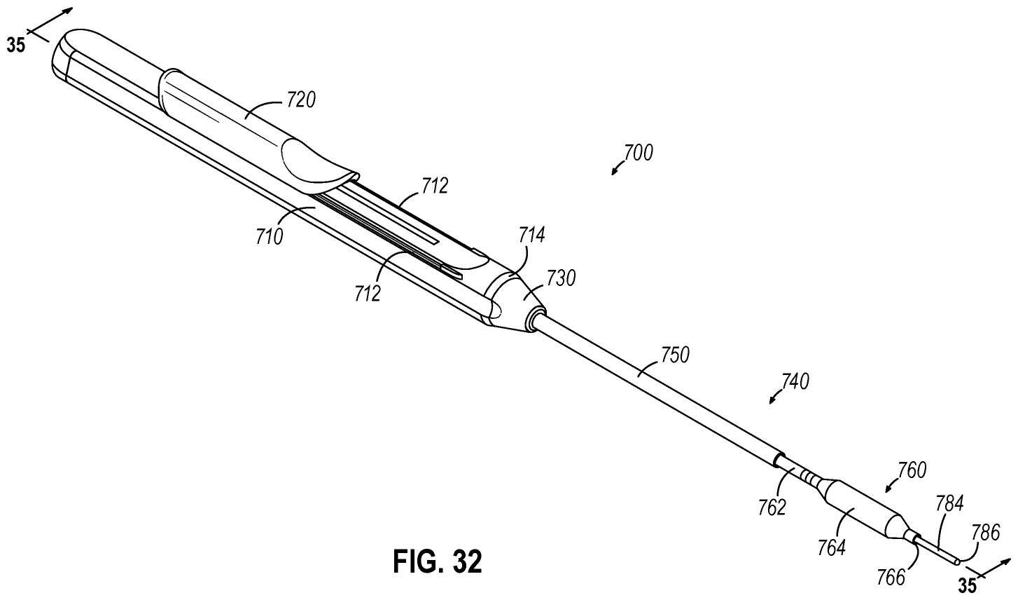

FIG. 32 shows an example of a dilation instrument (700) having a handle (710), a guide rail actuation knob (730), and a shaft assembly (740). A dilation catheter actuator (720) is slidably supported on a pair of longitudinally extending rails (712) that protrude outwardly from handle (710). Dilation catheter actuator (720) is configured to slide longitudinally along rails (712) to drive longitudinal movement of a dilation catheter assembly (760) relative to handle (710). By way of example only, a dilation catheter actuation mechanism like the dilation catheter actuation mechanism described above in connection with FIGS. 16A-28E may be provided within handle (710), with dilation catheter actuator (720) being operable in a manner similar to that described above with respect to actuator (320). Alternatively, any other suitable kind of mechanism may provide translation of dilation catheter assembly (760) relative to handle (710) in response to translation of dilation catheter actuator (720) along rails (712). Moreover, variations of instrument (700) may include any other suitable kind of actuator may be provided in lieu of the depicted dilation catheter actuator (720).

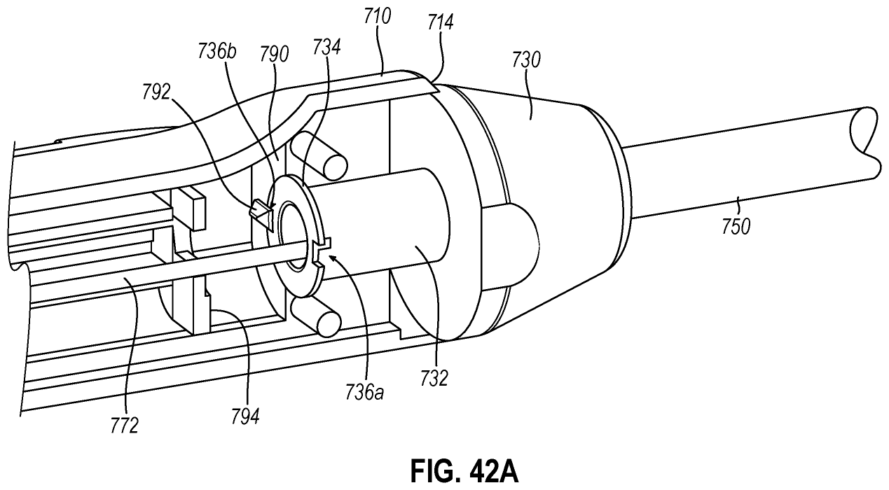

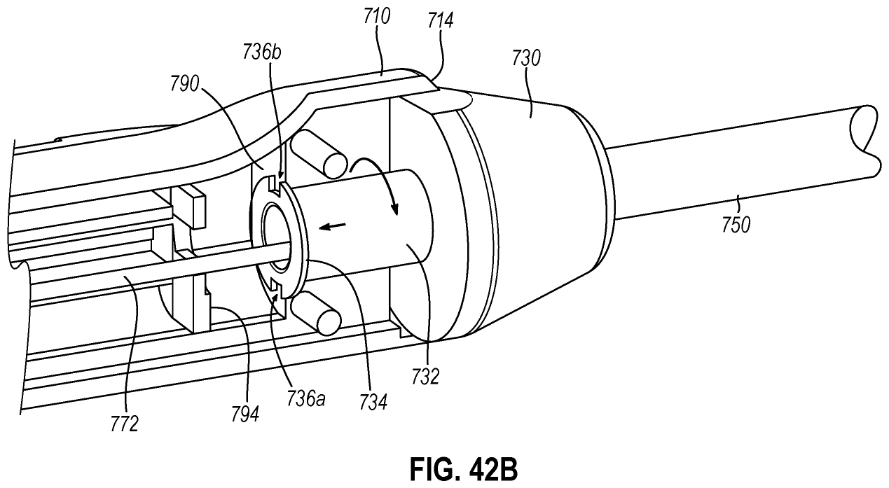

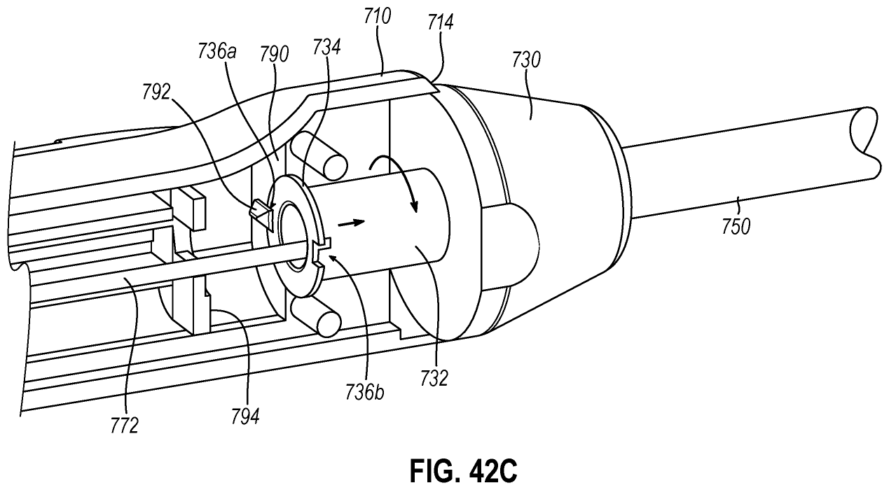

Guide rail actuation knob (730) is positioned distally relative to the distal end (714) of handle (710) in this example. Guide rail actuation knob (730) is movably coupled with handle (710) such that guide rail actuation knob (730) is operable to translate and rotate relative to handle (710) as will be described in greater detail below with reference to FIGS. 42A-42C. In some other variations, guide rail actuation knob (730) is only operable to translate relative to handle (710). In still other variations, guide rail actuation knob (730) is only operable to rotate relative to handle (710).

Shaft assembly (740) of the present example includes an outer sheath (750), dilation catheter assembly (760), and a guide rail assembly (780). While not shown, some versions of shaft assembly (740) may also include a guide element that is configured and operable like guide element (510) described above. For instance, such a guide element may be slidably disposed in guide rail (784) of guide rail assembly (780); and dilation catheter actuator (720) may provide staged actuation of the guide element and dilation catheter assembly (760) in a manner similar to that described above with reference to FIGS. 27A-28E. Alternatively, the guide element may be actuated in any other suitable fashion. Moreover, some versions of shaft assembly (740) may lack a guide element that is similar to guide element (510).

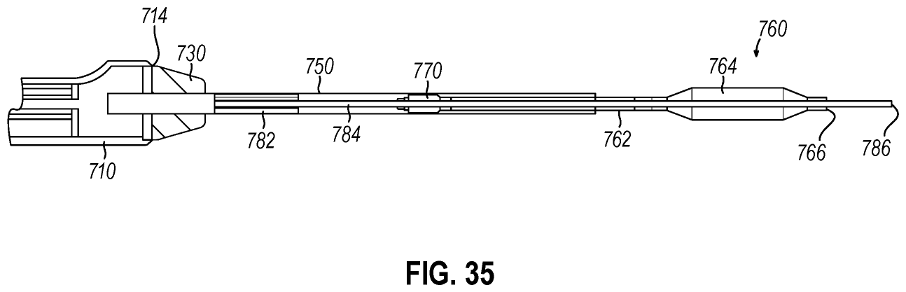

Outer sheath (750) comprises a rigid tube that is rigidly secured to guide rail actuation knob (730), such that outer sheath (750) will translate and rotate unitarily with guide rail actuation knob (730) in this example. As best seen in FIG. 35, portions of dilation catheter assembly (760) and guide rail assembly (780) enter a portion outer sheath (750). While a portion of dilation catheter assembly (760) extends proximally past the proximal end of outer sheath (750) and enters handle (710) via guide rail actuation knob (730), guide rail assembly (780) proximally terminates within outer sheath (750) at a longitudinal position that is distal to the distal end of guide rail actuation knob (730) and distal to distal end (714) of handle (710). Thus, no portion of guide rail assembly (780) enters guide rail actuation knob (730) or handle (710) in this example.

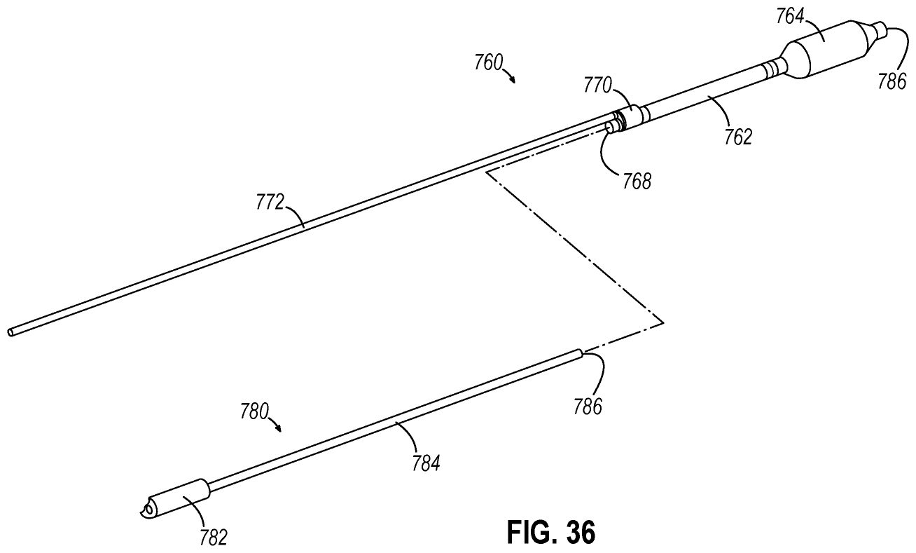

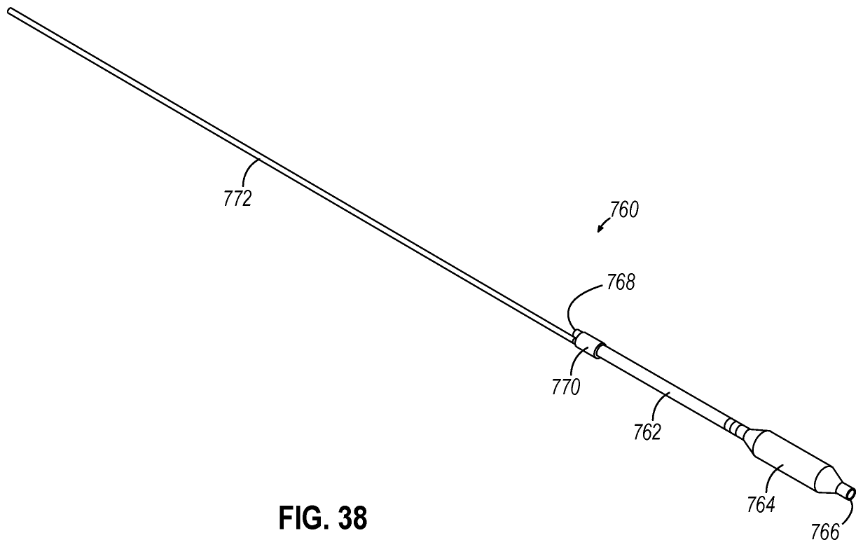

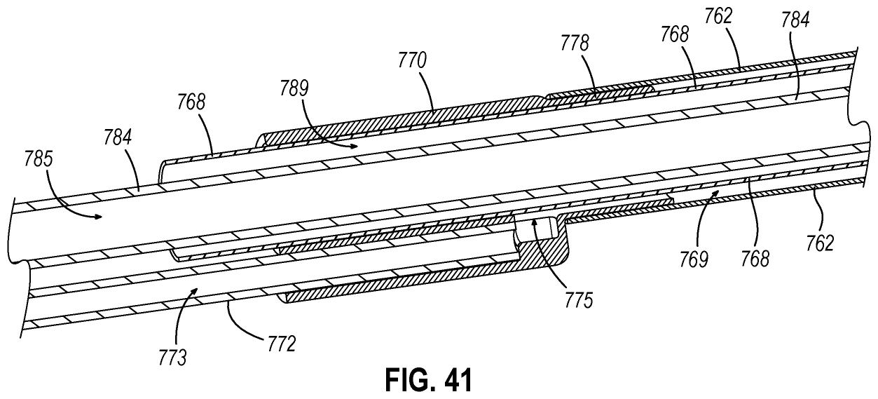

As best seen in FIGS. 36 and 38-41, dilation catheter assembly (760) of this example includes an outer shaft (762), a balloon (764), an inner shaft (768), a manifold (770), and a drive rod (772). Balloon (764) may comprise a non-extensible material and may be sized and configured to fit within a targeted anatomical passageway while in the deflated state; then dilate the targeted anatomical passageway while in the inflated state. As best seen in FIG. 41, inner shaft (768) is disposed within outer shaft (762), and shafts (762, 768) together define a gap (769) between the inner diameter of outer shaft (762) and the outer diameter of inner shaft (768). This gap (769) provides a pathway for communication of fluid to and from balloon (764) for inflation and deflation, respectively, of balloon (764).

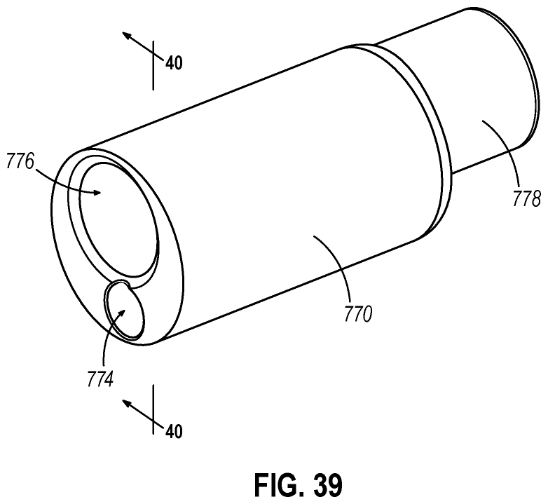

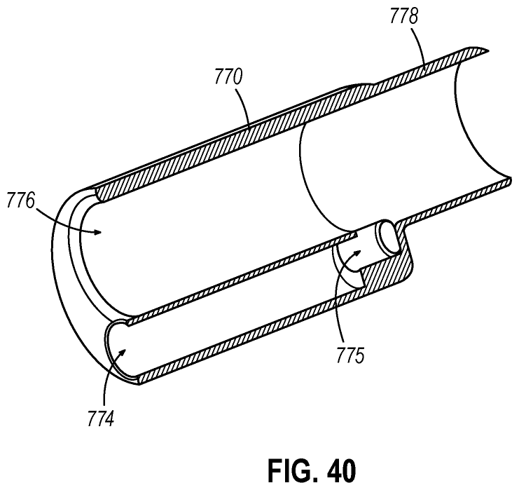

As best seen in FIGS. 39-40, manifold (770) of the present example includes an upper bore (776), a lower bore (774), a transverse opening (775), and a distal nose portion (778). Transverse opening (775) is formed at the distal end of lower bore (776) and provides a path for fluid communication between bores (774, 776). As best seen in FIG. 41, the proximal end of outer shaft (762) is fixedly secured to distal nose portion (778) (e.g., via adhesive, etc.), such that the proximal end of outer shaft (762) terminates at the proximal end of distal nose portion (778). As also shown in FIG. 41, inner shaft (768) is disposed within upper bore (776) and is fixedly secured to manifold (770) (e.g., via adhesive, etc.). While the proximal end of inner shaft (768) protrudes from and terminates proximal to the proximal end of manifold (770) in this example, the proximal end of inner shaft (768) may alternatively be flush with or distal to the proximal end of manifold (770) in other variations.

As also shown in FIG. 41, the distal end of drive rod (772) is disposed within lower bore (774) and is fixedly secured to manifold (770) (e.g., via adhesive, etc.). The proximal end of drive rod (772) is secured to a dilation catheter actuation assembly within handle (710), whereby dilation catheter actuator (720) is operable to drive longitudinal movement of drive rod (772) relative to handle (710). Since drive rod (772), inner shaft (768), and outer shaft (762) are all fixedly secured to manifold (770), manifold (770) is operable to transmit longitudinal motion from drive rod (772) to shafts (762, 768). Since balloon (764) is fixedly secured to shafts (762, 768) all of these components of dilation catheter assembly (760) translate together unitarily. The proximal end of drive rod (772) is also coupled with a source of inflation fluid (e.g., saline, etc.) and defines a lumen (773) that is in fluid communication with transverse opening (775) as shown in FIG. 41. Transverse opening (775) is also in fluid communication with gap (769) between the inner diameter of outer shaft (762) and the outer diameter of inner shaft (768). Thus, inflation fluid may be communicated from the fluid source to balloon (764) via lumen (773), transverse opening (775), and gap (769).

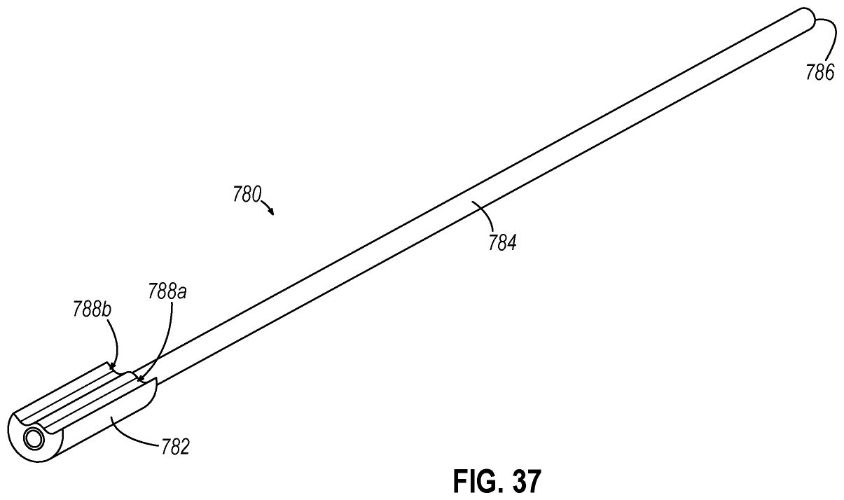

As best seen in FIGS. 36-37, guide rail assembly (780) of the present example includes a proximal body (782) and guide rail (784). The proximal end of guide rail (784) is fixedly secured within body (782), such that guide rail (784) and body (782) will move together unitarily. Body (782) includes a pair of longitudinally extending recesses (788a, 788b), as will be described in greater detail below. Body (782) is fixedly secured within outer sheath (750), as best seen in FIG. 35, such that body (782) and outer sheath (750) will also move together unitarily. As noted above, outer sheath (750) is fixedly secured to guide rail actuation knob (730), such that outer sheath (750) will move unitarily with guide rail actuation knob (730). Thus, guide rail actuation knob (730), outer sheath (750), body (782), and guide rail (784) all move together unitarily in this example. Referring back to FIG. 35, body (782) is positioned distally relative to the distal end of guide rail actuation knob (730) and distal to distal end (714) of handle (710). Even in scenarios where body (782) translates proximally relative to handle (710) in response to proximal retraction of guide rail actuation knob (730) as described in greater detail below, neither any portion of body (782) nor any portion of guide rail (784) enters handle (710) or is otherwise positioned proximally relative to distal end (714) of handle (710) at any stage of operation of instrument (700) in this example.

Guide rail (784) of the present example is malleable and has an atraumatic distal tip (786). In some versions, distal tip (786) is dome shaped. In some other versions, distal tip (786) is enlarged (e.g., configured as a ball tip or blueberry tip, etc.). The malleability of guide rail (784) allows guide rail (784) to be bent to a desired bend angle before being inserted into the head of the patient. The malleability of guide rail (784) may allow guide rail (784) to maintain the bend angle while guide rail (784) is disposed in the head of the patient, including while dilation catheter assembly (760) is advanced distally relative to guide rail (784). Such operability of guide rail (784) may promote access by dilation catheter assembly (760) to various locations within the head (H) of a patient (P), such as the maxillary sinus ostium, the frontal recess, the sphenoid sinus ostium, the Eustachian tube, etc., based on the selected bend angle. By way of example only, the bending of guide rail (784) may be performed in accordance with at least some of the teachings of U.S. Pat. No. 11,013,897, entitled “Apparatus for Bending Malleable Guide of Surgical Instrument,” issued May 25, 2021, the disclosure of which is incorporated by reference herein, in its entirety.

As shown in FIGS. 36 and 41, guide rail (784) is slidably disposed within a lumen (789) defined by inner shaft (768), such that manifold (770) and shafts (762, 768) are slidably disposed along guide rail (784). As also shown in FIG. 41, guide rail (784) of the present example also defines a lumen (785). In versions of instrument (700) that include a guide element similar to guide element (510) described above, such a guide element may be disposed in lumen (785) of guide rail (784). Alternatively, any other suitable kind(s) of element may be disposed in lumen (785) of guide rail (784). In some other variations, guide rail (784) may be solid such that lumen (785) is omitted.

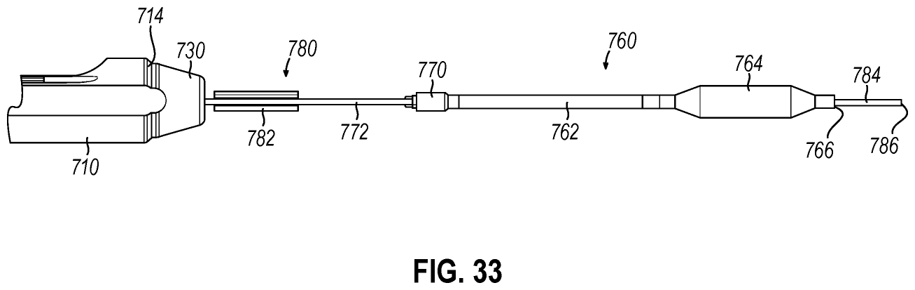

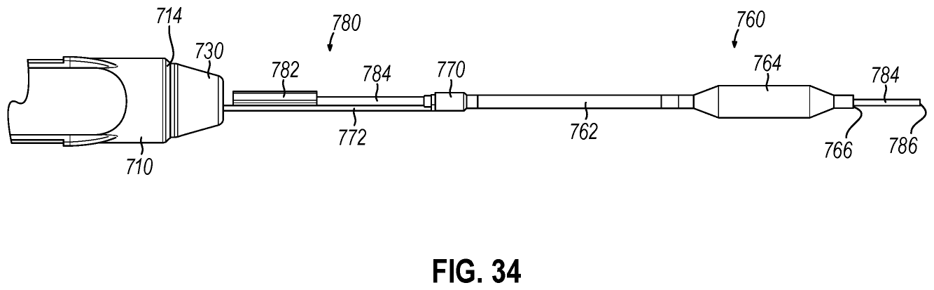

As shown in FIGS. 32-35, shaft assembly (740) is configured such that the distal end (766) of dilation catheter assembly (760) is proximal to distal tip (786) of guide rail (784) when dilation catheter assembly (760) is in a proximal position. However, when dilation catheter actuator (720) is driven distally along rails (712), thereby driving dilation catheter assembly (760) distally, distal end (766) of dilation catheter assembly (760) may be positioned distal to distal tip (786) of guide rail (784).

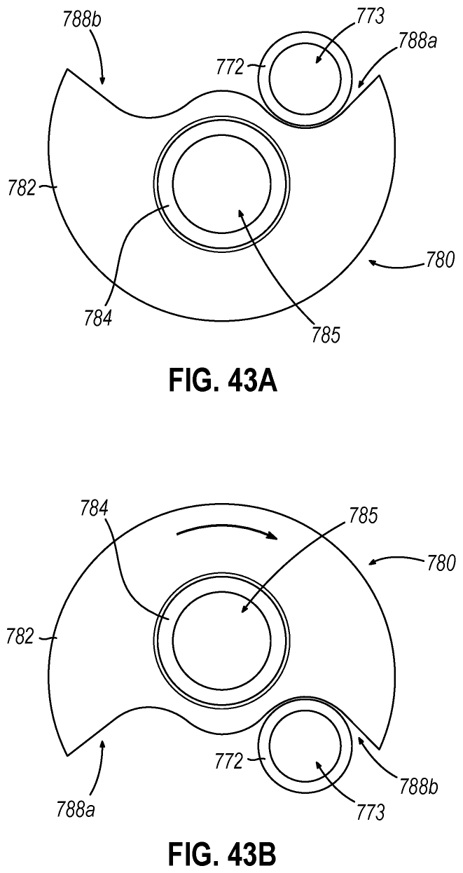

FIGS. 42A-42C and 43A-43B show an example of how guide rail actuation knob (730) may be actuated to rotate guide rail assembly (780) about a longitudinally extending axis, to thereby reorient distal tip (786) about that axis. As shown in FIGS. 42A-42C, a barrel (732) extends proximally from guide rail actuation knob (730) and has a bore in which drive rod (772) is freely received. An annular flange (734) is positioned at the proximal end of barrel (732).

Flange (734) includes a first notch (736a) and a second notch (736b). As also shown in FIGS. 42A-42C, handle (710) includes a distal inner wall (790) and a proximal inner wall (794). While only one housing half of handle (710) is shown in FIGS. 42A-42C, it should be understood that distal inner wall (790) and/or proximal inner wall (794) may span across both housing halves of handle (710). Distal inner wall (790) includes a proximal projection (792) that is sized and configured for receipt in a notch (736a, 736b). In versions where distal inner wall (790) spans across both housing halves of handle (710), a second projection (792) may be similarly positioned on the region of distal inner wall (790) on the other housing half of handle (710). It should be understood that any suitable number of projections (792) may be provided in handle (710), in any suitable arrangement. Similarly, flange (734) may have any suitable number of notches (736a, 736b) in any suitable arrangement.

While not shown in the present example, a resilient member (e.g., coil spring, wave spring stack, etc.) may be longitudinally interposed between the proximal face of flange (734) and the distal face of proximal inner wall (794). Such a resilient member may impart a distally oriented bias on flange (734), thereby imparting a distally oriented bias on guide rail actuation knob (730), outer sheath (750), body (782), and guide rail (784) relative to handle (710). This distally oriented bias on flange (734) may also press the distal face of flange (734) against the proximal face of distal inner wall (790), with projection (792) being received in a notch (736b), as shown in FIG. 42A. Projection (792) and notch (736b) may thus cooperate to provide a detent that substantially maintains the angular position of guide rail actuation knob (730), outer sheath (750), body (782), and guide rail (784) about the longitudinal axis, relative to handle (710).

To re-orient guide rail (784) about the longitudinal axis, the operator may drive guide rail actuation knob (730) proximally relative to handle (710) decouple projection (792) and the corresponding notch (736a, 736b), then rotate guide rail actuation knob (730) about the longitudinal axis relative to handle (710) as shown in FIG. 42B. In the present example, the operator rotates guide rail actuation knob (730) until notch (736a) is aligned with projection (792). The operator then ceases rotation guide rail actuation knob (730) relative to handle (710), and releases guide rail actuation knob (730) to allow the resilient member (not shown) to drive guide rail actuation knob (730) distally. Guide rail actuation knob (730) eventually reaches a point where the distal face of flange (734) is pressed against the proximal face of distal inner wall (790), with projection (792) being received in a notch (736a) as shown in FIG. 42C. Thus, in the sequence shown in FIGS. 42A-42C, guide rail actuation knob (730), outer sheath (750), body (782), and guide rail (784) have been rotated about the longitudinal axis 180 degrees relative to handle (710). Projection (792) and notch (736a) cooperate to provide a detent that substantially maintains the angular position of guide rail actuation knob (730), outer sheath (750), body (782), and guide rail (784) about the longitudinal axis, relative to handle (710), in the state shown in FIG. 42C.

FIGS. 43A-43B show the change in relative positioning between body (782) of guide rail assembly (780) and drive rod (772) of dilation catheter assembly (760). In particular, the state shown in FIG. 43A corresponds to the state shown in FIG. 42A; while the state shown in FIG. 43B corresponds to the state shown in FIG. 42C. As can be seen, longitudinally extending recess (788a), accommodates drive rod (772) in the state shown in FIGS. 43A and 43B; while longitudinally extending recess (788b), accommodates drive rod (772) in the state shown in FIGS. 43B and 43C.

As described above, guide rail actuation knob (730), outer sheath (750), body (782), and guide rail (784) are rotatable about the longitudinal axis relative to handle (710) through an angular range of 180 degrees. In some other versions, instrument (700) is configured such that guide rail actuation knob (730), outer sheath (750), body (782), and guide rail (784) are rotatable about the longitudinal axis relative to handle (710) through an angular range that is larger or smaller than 180 degrees. Similarly, as described above, notches (736a, 736b) and projection (792) are configured to substantially maintain the angular position of guide rail actuation knob (730), outer sheath (750), body (782), and guide rail (784) relative to handle (710) at two different angular positions. In some other versions, instrument (700) is configured such that notches (736a, 736b) and projection (792) are configured to substantially maintain the angular position of guide rail actuation knob (730), outer sheath (750), body (782), and guide rail (784) relative to handle (710) at more than two different angular positions. In still other variations, instrument (700) may be configured such that guide rail (784) is not rotatable relative to handle (710).

### IV. Example of Guide Rail with Varying Young's Modulus

As noted above, guide rail (784) is malleable. In some versions, the entire length of guide rail (784) is malleable. In some other versions, a proximal region of guide rail (784) is fully rigid while only a distal region of guide rail (784) is malleable. In either scenario, it may be desirable to provide varying Young's modulus (i.e., resistance to buckling in axial compression) along one or more regions of guide rail (784). For instance, it may be desirable to provide a relatively low Young's modulus in the distal region of guide rail (784), which may facilitate bending to achieve desired bend angles for reaching different anatomical passageways with balloon (764); while providing a relatively high Young's modulus elsewhere along guide rail (784), to prevent inadvertent deflection (e.g., buckling) of guide rail (784). However, even in cases where a distal region of guide rail (784) provides a relatively low Young's modulus, it may be further desirable for that same distal region of guide rail (784) to avoid kinking when bent; and for that same distal region of guide rail (784) to sufficiently maintain a formed bend (e.g., avoid buckling) as that distal region of guide rail (784) is pushed into a small anatomical passageway (e.g., paranasal sinus ostium, Eustachian tube, etc.).

In the present example, guide rail (784) is initially provided with a Young's modulus that is consistent along the length of guide rail (784). A distal portion of guide rail (784) is then treated to reduce the Young's modulus along only the distal portion; while the rest of guide rail (784) maintains the initial Young's modulus. Also in the present example, guide rail (784) comprises stainless steel; and the treatment to reduce Young's modulus of the stainless steel comprises applying heat to the stainless steel.

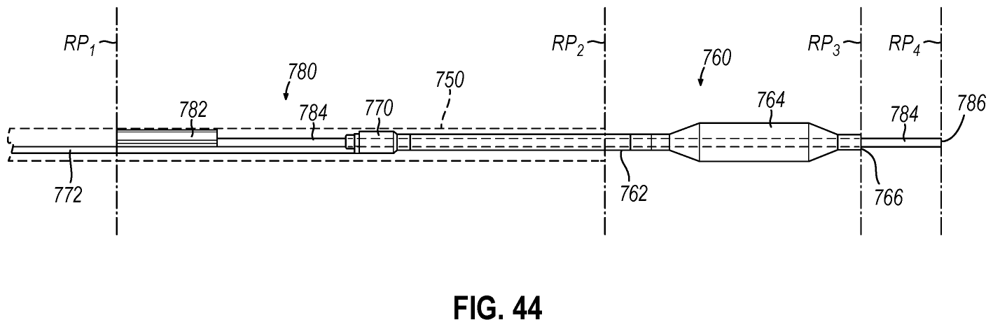

FIG. 44 shows components of shaft assembly (740) in relation to several reference planes (RP₁, RP₂, RP₃, RP₄), with outer sheath (750) shown in phantom. As shown, a first reference plane (RP₁) is positioned at the proximal end of guide rail (784); a second reference plane (RP₂) is positioned at the distal end of outer sheath (750); a third reference plane (RP₃) is positioned at distal end (766) of dilation catheter assembly (760) (when dilation catheter assembly (760) is in a proximally retracted position along guide rail (784)); and a fourth reference plane (RP₄) is positioned at distal tip (786) of guide rail (784). In this example, heat is applied primarily to the region of guide rail (784) extending between the third and fourth reference planes (RP₃, RP₄). This reduces the Young's modulus of the region of guide rail (784) extending between the third and fourth reference planes (RP₃, RP₄), thereby facilitating bending of the region of guide rail (784) extending between the third and fourth reference planes (RP₃, RP₄). However, this region of guide rail (784) extending between the third and fourth reference planes (RP₃, RP₄) still has sufficient strength to avoid kinking when bent; and to sufficiently maintain a formed bend (e.g., avoid buckling) as this region of guide rail (784) is pushed into a small anatomical passageway (e.g., paranasal sinus ostium, Eustachian tube, etc.).

In the present example, the application of heat to the region of guide rail (784) extending between the third and fourth reference planes (RP₃, RP₄) may incidentally also have some effect on the Young's modulus of the region of guide rail (784) extending between the second and third reference planes (RP₂, RP₃). To the extent that the Young's modulus of the region of guide rail (784) extending between the second and third reference planes (RP₂, RP₃) is slightly reduced relative to the initial Young's modulus of guide rail (784), the Young's modulus of the region of guide rail (784) extending between the second and third reference planes (RP₂, RP₃) may still be higher than the Young's modulus of the region of guide rail (784) extending between the third and fourth reference planes (RP₃, RP₄).

Also in the present example, the heat applied to the region of guide rail (784) extending between the third and fourth reference planes (RP₃, RP₄), and the effects of such heat, do not reach the region of guide rail (784) extending between the first and second reference planes (RP₁, RP₂). Thus, this region of guide rail (784) maintains the initial Young's modulus of guide rail (784), such that the Young's modulus of the region of guide rail (784) extending between the first and second reference planes (RP₁, RP₂) is higher than the Young's modulus of the region of guide rail (784) extending between the second and third reference planes (RP₂, RP₃); and substantially higher than the Young's modulus of the region of guide rail (784) extending between the third and fourth reference planes (RP₃, RP₄).

It should be understood that the precise position of the second and third reference planes (RP₂, RP₃) may vary. It should also be understood that the Young's modulus of guide rail (784) may not necessarily suddenly transition from one value to a substantially different value upon crossing the second or third reference plane (RP₂, RP₃). For instance, the Young's modulus may progressively increase along a spectrum in the region of guide rail (784) between the second and third reference planes (RP₂, RP₃). Thus, the region of guide rail (784) between the second and third reference planes (RP₂, RP₃) may be generally viewed as a transition region.

By way of further example only, the length of the region of guide rail (784) between the third and fourth reference planes (RP₃, RP₄) may be approximately 1 inch. By way of further example only, the region of guide rail (784) between the third and fourth reference planes (RP₃, RP₄) may have a Young's modulus ranging from approximately 20 N/mm² to approximately 70 N/mm²; or more particularly ranging from approximately 30 N/mm² to approximately 45 N/mm²; or more particularly approximately 40 N/mm.

### V. Second Example of Dilation Instrument with Adjustable Guide Rail, with Additional Translating Guide Tip Element Features

As noted above, it may be further desirable to achieve different longitudinal positions and/or angular positions in a malleable guide of a dilation instrument, to thereby facilitate access to different anatomical passageways. As also noted above, it may be desirable to provide an additional guide element (e.g., providing position sensing and/or transillumination) that is operable to translate in different stages relative to a dilation catheter. The following describes an example of a dilation instrument that provides a combination of an adjustable malleable guide and enhanced control over a translating guide element.

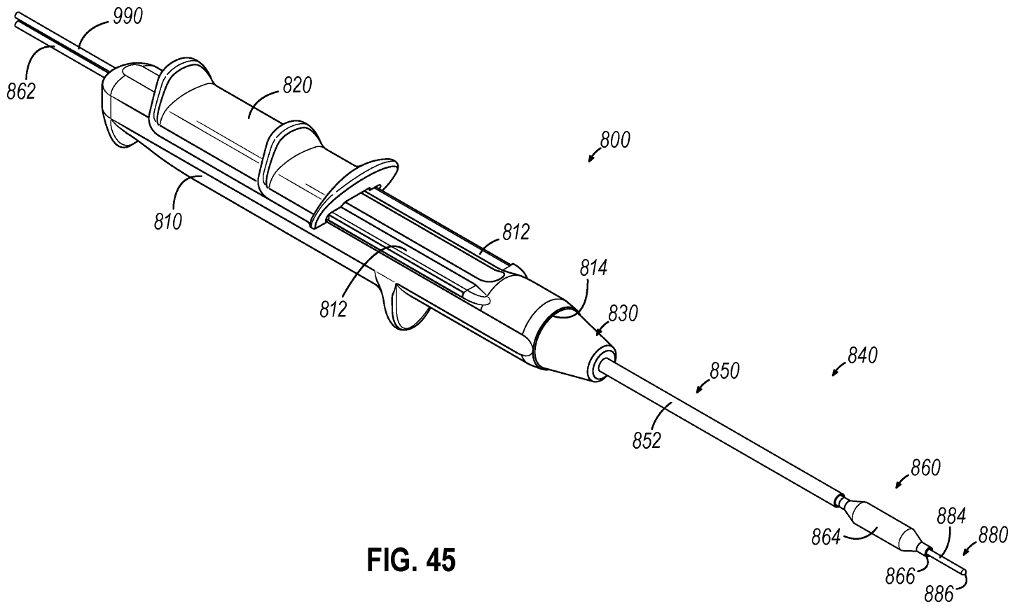

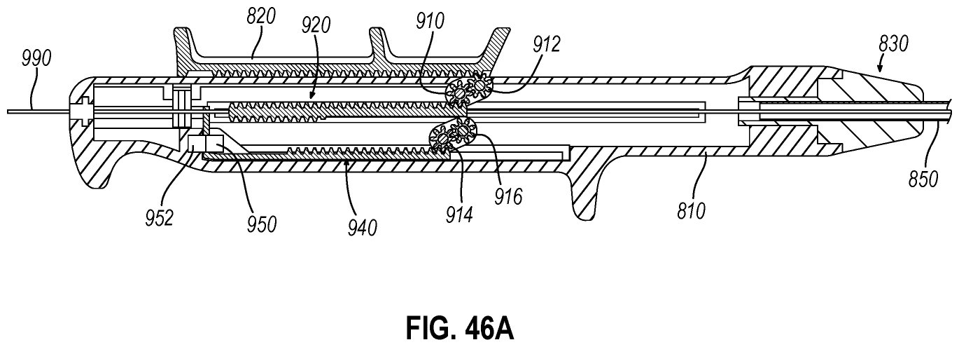

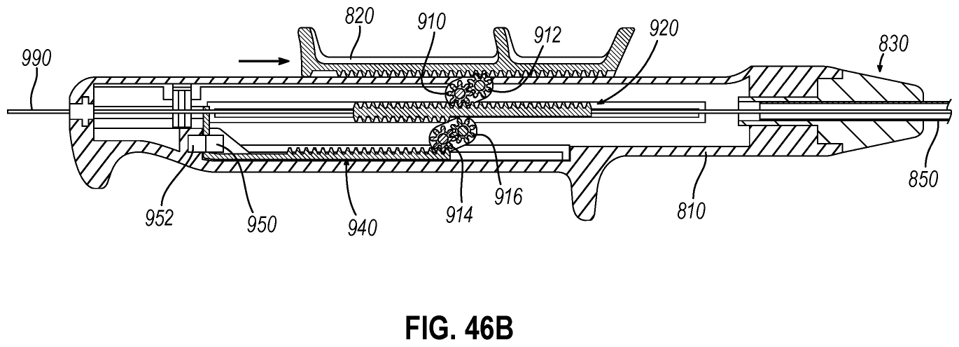

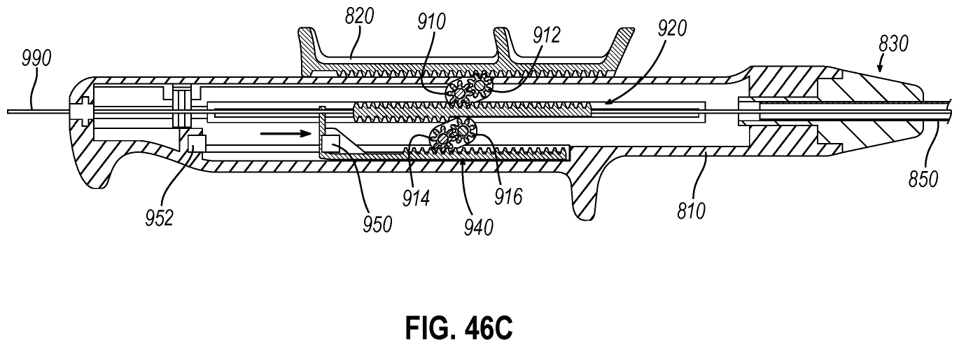

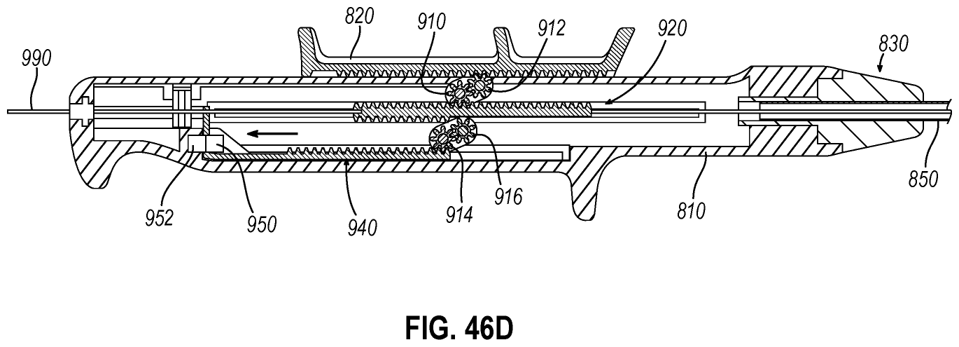

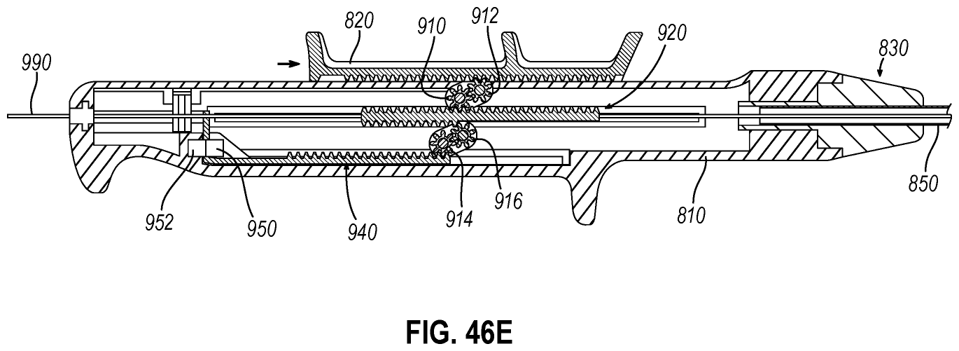

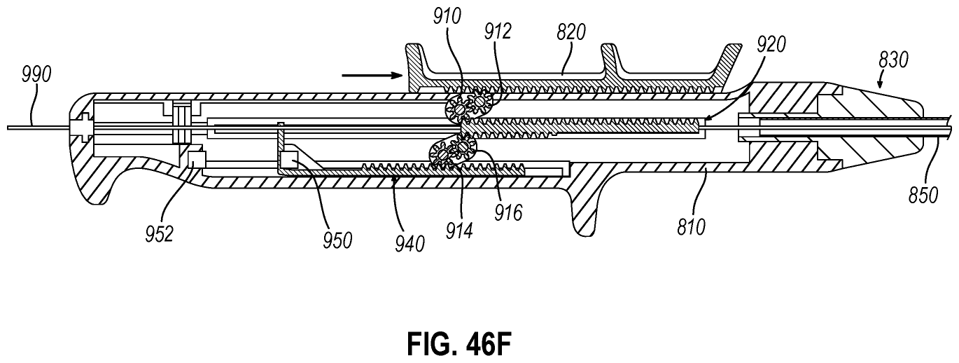

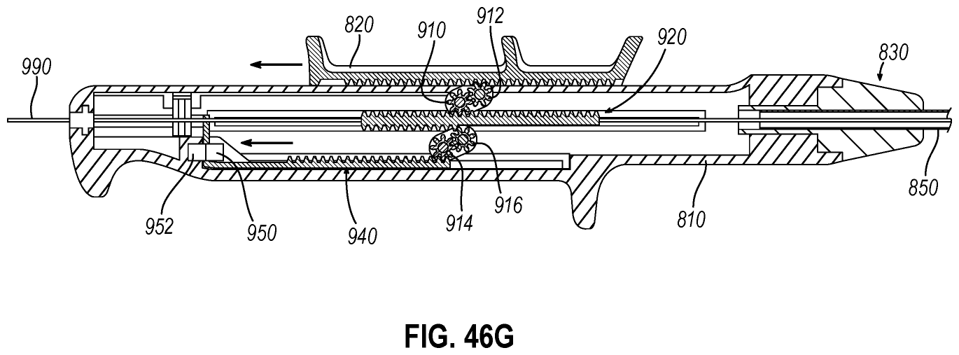

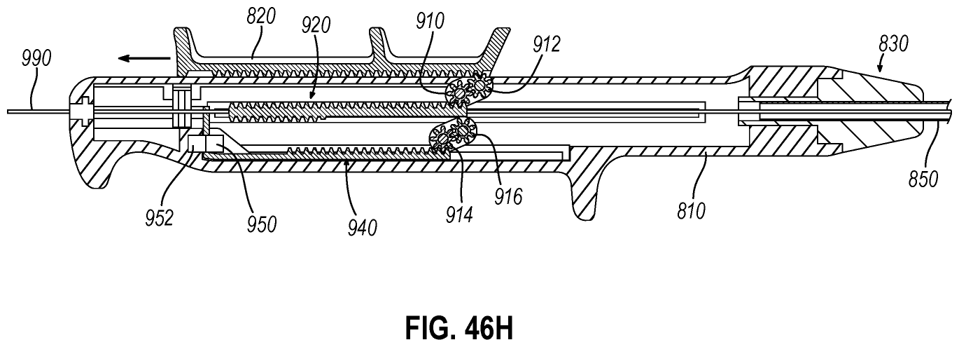

FIG. 45 shows an example of a dilation instrument (800) that is configured and operable like dilation instrument (700) of FIG. 32, except for the differences described below. Dilation instrument (800) of this example includes a handle (810), a guide rail actuation knob (830), and a shaft assembly (840). A dilation catheter actuator (820) is slidably supported on a pair of longitudinally extending rails (812) that protrude outwardly from handle (810). Dilation catheter actuator (820) is configured to slide longitudinally along rails (812) to drive longitudinal movement of a dilation catheter assembly (860) relative to handle (810). By way of example only, and as described in greater detail below with reference to FIGS. 46A-46H, a dilation catheter actuation mechanism like the dilation catheter actuation mechanism described above in connection with FIGS. 16A-28E may be provided within handle (810), with dilation catheter actuator (820) being operable in a manner similar to that described above with respect to actuator (320). Alternatively, any other suitable kind of mechanism may provide translation of dilation catheter assembly (860) relative to handle (810) in response to translation of dilation catheter actuator (820) along rails (812). Moreover, variations of instrument (800) may include any other suitable kind of actuator may be provided in lieu of the depicted dilation catheter actuator (820).

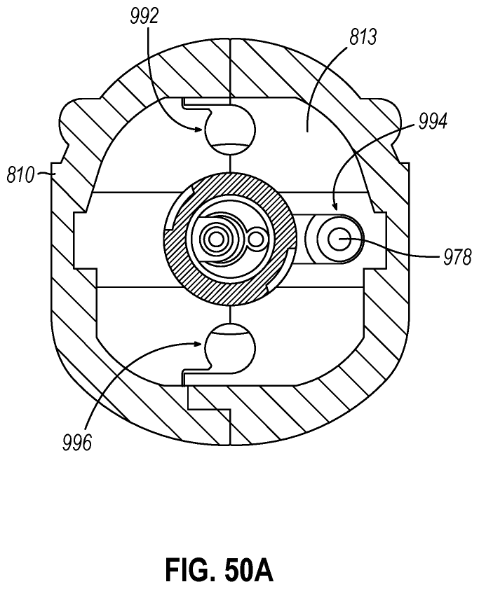

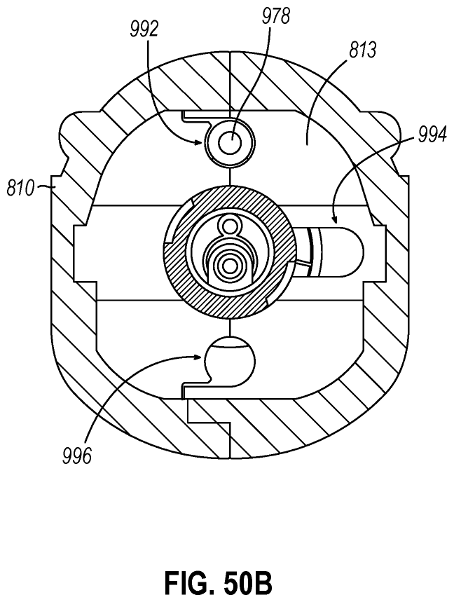

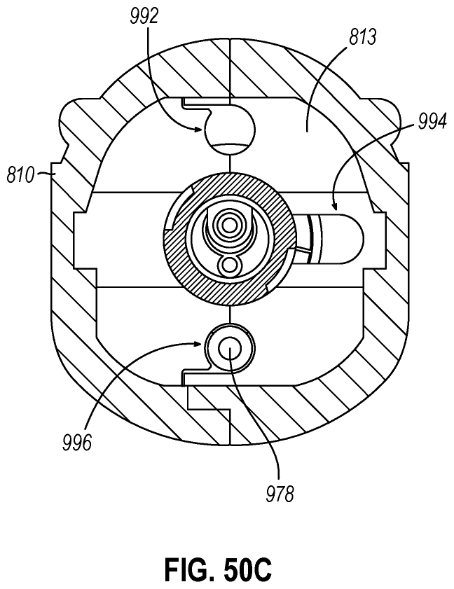

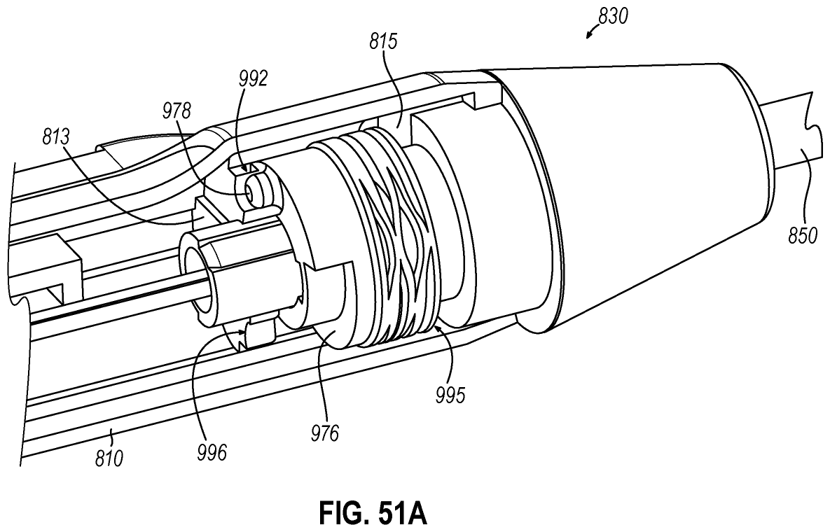

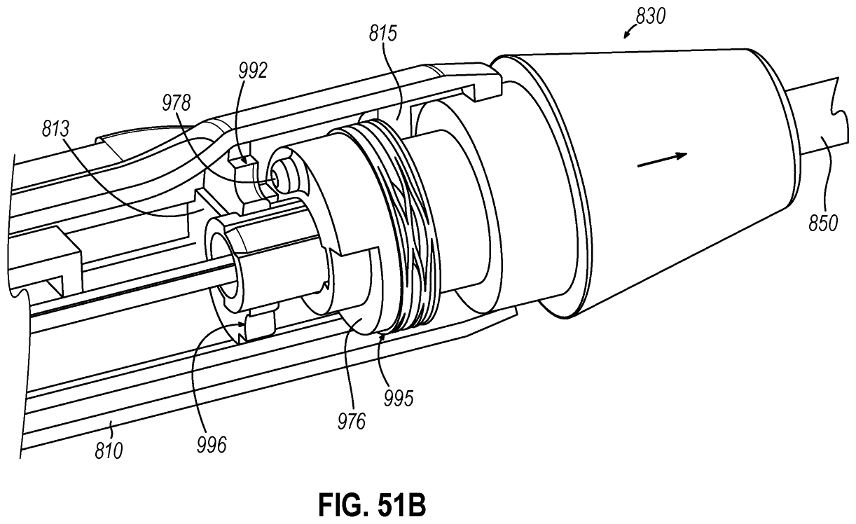

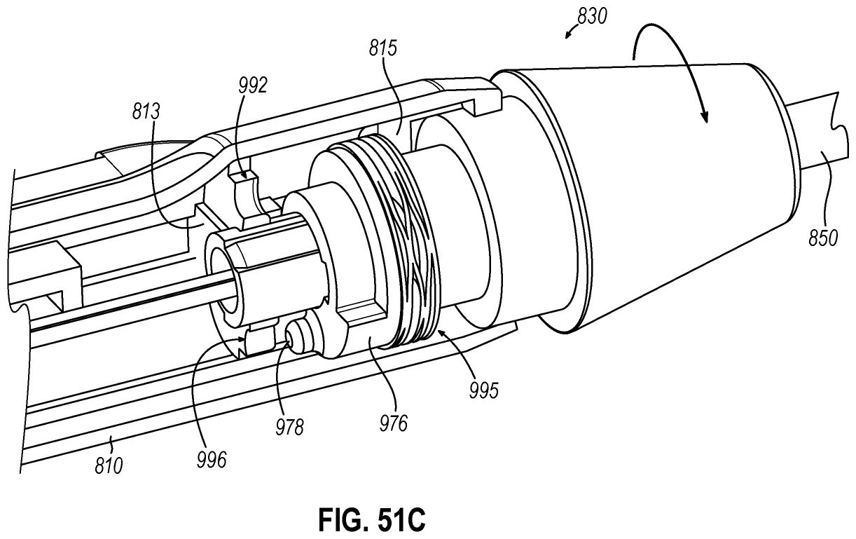

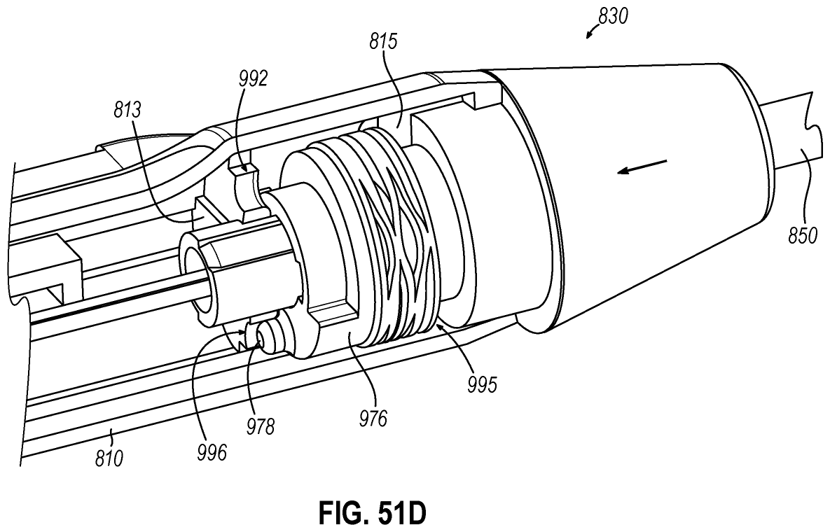

Guide rail actuation knob (830) is positioned distally relative to the distal end (814) of handle (810) in this example. Guide rail actuation knob (830) is movably coupled with handle (810) such that guide rail actuation knob (830) is operable to translate and rotate relative to handle (810) as will be described in greater detail below with reference to FIGS. 50A-51D. In some other variations, guide rail actuation knob (830) is only operable to translate relative to handle (810). In still other variations, guide rail actuation knob (830) is only operable to rotate relative to handle (810). Some components of shaft assembly (840) are fixedly secured to guide rail actuation knob (830), such that these components of shaft assembly (840) will rotate and translate with guide rail actuation knob (830) relative to handle (810).

Shaft assembly (840) of the present example includes an outer sheath (850), dilation catheter assembly (860), a guide rail assembly (880), and a guide element (990). Outer sheath (850) is fixedly secured to guide rail actuation knob (830); and may be configured and operable like outer sheath (750) described above. Dilation catheter assembly (860) may be configured and operable like dilation catheter assembly (760) described above; and includes an outer shaft (862) and a balloon (864). While not shown, dilation catheter assembly (860) further includes an inner shaft, a manifold, and a drive rod; which are configured and operable like an inner shaft (768), a manifold (770), and a drive rod (772), respectively. Guide rail assembly (880) may be configured and operable like guide rail assembly (780) described above; and includes a malleable guide rail (884) with an atraumatic distal tip (886). In the present example, no portion of guide rail assembly (780) enters handle (810) or is otherwise positioned proximally relative to distal end (814) of handle (810) at any stage of operation of instrument (800) in this example. Guide rail assembly (880) is fixedly secured to outer sheath (850). Guide rail assembly (880) is thus fixedly secured relative to guide rail actuation knob (830), such that guide rail assembly (880) and outer sheath (850) will rotate and translate with guide rail actuation knob (830) relative to handle (810).

Guide element (990) may be configured and operable like guide element (510) described above, such that guide element (990) may have a distal tip with an indicator element, like indicator element (516), that is configured to indicate the position of the distal tip in three-dimensional space. In the present example, the indicator element includes an illuminating feature that is operable to project light outwardly to thereby provide transillumination through the skin of the patient as described above. In addition, or in the alternative, the indicator element may include one or more position sensors (e.g., coils) that provide position-indicative signals to an image-guided surgery system. As described elsewhere herein, guide rail (884) is disposed within outer shaft (862) of dilation catheter assembly (860), such that dilation catheter assembly (860) translates about guide rail (884) in this example; while guide element (990) is disposed within guide rail (884), such that guide element (990) translates within guide rail (884) in this example.

FIGS. 46A-46G show components that may be used to actuate dilation catheter assembly (860) and guide element (990) relative to handle (810). As shown, a set of rack members (920, 940) and pinions (910, 912, 914, 916) are disposed within handle (810). Rack members (920, 940) may be configured and operable like rack members (420, 440), as described above, respectively. Pinions (910, 912, 914, 916) may be configured and operable like pinions (410, 412, 414, 416), as described above, respectively. Pinion (912) is in meshing engagement with teeth on the underside of dilation catheter actuator (820); and with teeth of pinion (910). Pinion (910) is also in meshing engagement with teeth on the upper side of rack member (920). Dilation catheter assembly (860) is coupled with rack member (920), such that dilation catheter assembly (860) will translate unitarily with rack member (920) relative to handle (810). Thus, translation of dilation catheter actuator (820) along handle (810) will drive corresponding translation of dilation catheter assembly (860) relative to handle (810) via pinions (910, 912) and rack member (920).

Pinion (916) is positioned to mesh with teeth on the underside of rack member (920), depending on the longitudinal position of rack member (920) within handle (810) as described below. Pinion (916) is also in meshing engagement with pinion (914), which is also in meshing engagement with teeth on an upper side of rack member (940). Guide element (990) is coupled with rack member (940), such that guide element (990) will translate unitarily with rack member (940) relative to handle (810).

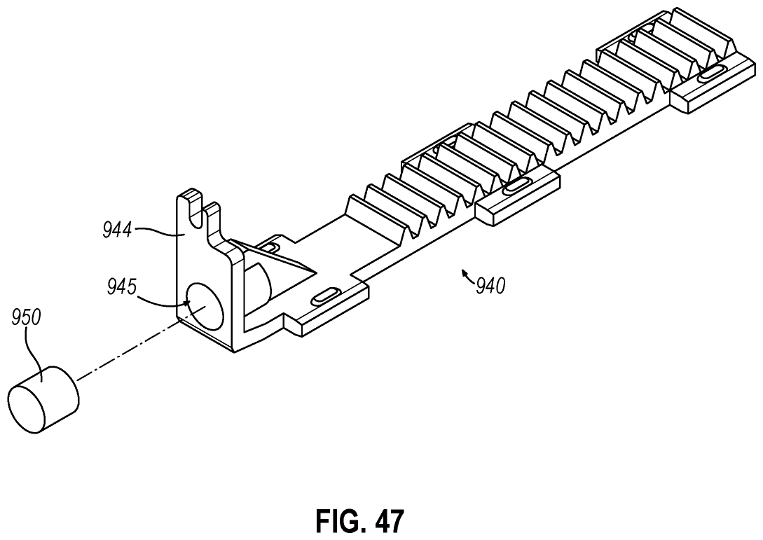

In the present example, and as best shown in FIG. 47, a permanent magnet (950) is disposed in a proximally facing recess (945) in proximal upright portion (944) of rack ember (940). As best shown in FIGS. 46A-46H, a permanent magnet (952) is fixedly secured within an interior region of handle (810). The pole of permanent magnet (950) facing permanent magnet (952) is opposite to the pole of permanent magnet (952) facing permanent magnet (950). Magnets (950, 952) are also positioned along a shared axis. Thus, when magnet (950) is within sufficient proximity to magnet (952) (e.g., as shown in FIGS. 46A-46B, 46D-46E, and 46G-46H), magnets (950, 952) may be magnetically coupled. Such magnetic coupling may substantially prevent inadvertent distal movement of rack member (940) relative to handle (810), while also allowing intentional distal movement of rack member (940) relative to handle (810), during operation of dilation instrument (800) as described below. By way of example only, such inadvertent distal movement of rack member (940) relative to handle (810) might otherwise occur when dilation catheter assembly (860) is translated relative to handle (810) during a stage of operation where it is intended for guide element (990) to remain longitudinally stationary relative to handle (810), such as when there is friction between one or more components associated with movement of dilation catheter assembly (860) and one or more components associated with movement of guide element (990). By way of further example only, such inadvertent distal movement of rack member (940) relative to handle (810) might otherwise occur when an operator inadvertently engages a portion of guide element (990) protruding proximally from handle (810).

In some variations, a ferrous element (e.g., a piece of steel, etc.) is used to replace one of permanent magnets (950, 952). In such variations, magnetic attraction between the ferrous element and the one permanent magnet (950, 952) may provide the same effect of substantially preventing inadvertent distal movement of rack member (940) relative to handle (810), while also allowing intentional distal movement of rack member (940) relative to handle (810), during operation of dilation instrument (800). As yet another variation, mechanical detent features, latches, or other retention features may be used in place of permanent magnets (950, 952) to substantially prevent inadvertent distal movement of rack member (940) relative to handle (810), while also allowing intentional distal movement of rack member (940) relative to handle (810), during operation of dilation instrument (800).

### A. Example of Staged Operation of Guide Element and Dilation Catheter Assembly

FIG. 46A shows an example of a first stage of operation that is similar to the stage of operation shown in FIGS. 27A and 28A, as described above. At this stage of operation dilation catheter assembly (860) is in a proximal position. Dilation catheter actuator (820) and each rack member (820, 940) are proximally positioned relative to handle (810) at this stage. The distal tip of guide element (990) is positioned at distal tip (886) of guide rail (884). Also in this state, distal end (866) of dilation catheter assembly (860) is spaced proximally from the distal tip of guide element (990) and distal tip (886) of guide rail (884). Thus, a distal region of guide rail (884) is exposed relative to dilation catheter assembly (860). The arrangement of shaft assembly (840) in this state may be similar to the arrangement of shaft assembly (301) shown in FIG. 28A and described above. In some cases, the distal region of guide rail (884) is in a bent configuration as described herein at the operational state shown in FIG. 46A. Shaft assembly (840) may be inserted into the patient (P) while the components of dilation instrument (800) are arranged as shown in FIG. 46A. As noted above, the magnetic coupling between magnets (950, 952) may substantially prevent inadvertent distal movement of rack member (940) relative to handle (810) while the components of dilation instrument (800) are arranged as shown in FIG. 46A, during insertion of shaft assembly (840) into the patient (P).

In the present example, guide element (990) has an illuminating feature at the distal tip of guide element (990), such that the operator may visually observe the face of the patient (P) to detect transillumination effects that verify appropriate positioning of the distal end of shaft assembly (840). For instance, if the operator is attempting to dilate an ostium of a maxillary sinus, the operator may visually observe the cheek of the patient (P) over the maxillary sinus cavity of the targeted ostium. When the cheek is transilluminated by the illuminating feature at the distal tip of guide element (990), such transillumination may visually indicate that the distal portion of shaft assembly (840) has been appropriately positioned at the targeted maxillary sinus ostium. As another example, if the operator is attempting to dilate a frontal recess, the operator may visually observe the forehead cheek of the patient over the frontal sinus cavity of the targeted frontal recess. When the forehead is transilluminated by the illuminating feature at the distal tip of guide element (990), such transillumination may visually indicate that the distal portion of shaft assembly (840) has been appropriately positioned at the targeted frontal recess.

In some cases, after shaft assembly (840) is initially inserted into the patient (P) and the operator observes transillumination at the expected region (e.g., through the cheek, through the forehead, etc.), the operator may wish to further confirm that shaft assembly (840) has appropriately reached the desired region near the targeted ostium, recess, or other passageway. To that end, the operator may wish to independently advance guide element (990) through the targeted passageway (e.g., maxillary sinus ostium or frontal recess, etc.) and into the cavity (e.g., maxillary sinus cavity or frontal sinus cavity) to observe additional transillumination effects indicating successful passage of guide element (990) through the targeted passageway and into the cavity. This additional advancement of guide element (990) may be provided in accordance with the operational states shown in FIGS. 46B-46C.

As shown in the transition from the state shown in FIG. 46A to the state shown in FIG. 46B, the operator may first advance dilation catheter actuator (820) distally to a first intermediate position, to provide clearance for independent longitudinal movement of rack member (940). With dilation catheter actuator (820) in the first intermediate position, rack member (920) is also in a first intermediate position. With rack member (920) at this first intermediate position, the teeth on the underside of rack member (920) are not yet engaged with pinion (916), such that pinions (914, 916) may rotate freely within handle (810) when rack member (920) is at the first intermediate position shown in FIG. 46B. In some versions, handle (810) and dilation catheter actuator (820) include complementary detent features that provide tactile feedback to the operator to indicate when dilation catheter actuator (820) has reached the first intermediate position shown in FIG. 46B. In addition, or in the alternative, handle (810) and dilation catheter actuator (820) may include complementary indicator features that visually indicate to the operator to indicate when dilation catheter actuator (820) has reached the first intermediate position shown in FIG. 46B. It should be understood that, in the operational state shown in FIG. 46B, the magnetic coupling between magnets (950, 952) may continue to substantially prevent inadvertent distal movement of rack member (940) relative to handle (810).

After dilation catheter actuator (820) and rack member (920) have reached the respective first intermediate positions shown in FIG. 46B, the operator may then grasp a portion of guide element (990) protruding proximally from handle (810) and thereby push guide element (990) distally relative to handle (810), as is shown in the transition from the state shown in FIG. 46B to the state shown in FIG. 46C. As the operator pushes guide element (990) distally relative to handle (810), this pushes rack member (940) distally within handle (810). It should be understood that the distal forces imparted on guide element (990) by the operator pushing on the proximal portion of guide element (990) protruding proximally from handle (810) may suffice to overcome the magnetic coupling between magnets (950, 952). The distal movement of rack member (940) causes both pinions (914, 916) to spin freely or “freewheel” within handle (810). The corresponding distal movement of guide element (990) also urges the distal tip of guide element (990) distally relative to distal tip (886) of guide rail (884).

If distal tip (886) of guide rail (884) is appropriately positioned at the targeted anatomical passageway (e.g., maxillary sinus ostium, frontal recess, etc.) during the transition from the state shown in FIG. 46B to the state shown in FIG. 46C, the distally advanced guide element (990) will traverse the targeted anatomical passageway and enter the adjacent cavity (e.g., maxillary sinus cavity, frontal sinus cavity, etc.). The entry of guide element (990) into the cavity will be visually observable through the corresponding transillumination effects. For instance, the operator may observe the source of light from the distal tip of guide element (990) moving along the cheek or forehead of the patient as guide element (990) moves within the maxillary sinus cavity or frontal recess. If such effects are not observed, then the absence of such effects may indicate that distal tip (886) of guide rail (884) is not appropriately positioned at the targeted anatomical passageway.

After independently advancing guide element (990) to confirm whether distal tip (886) of guide rail (884) is appropriately positioned at the targeted anatomical passageway, the operator may continue to grasp the portion of guide element (990) protruding proximally from handle (810) and thereby pull guide element (990) proximally relative to handle (810), as is shown in the transition from the state shown in FIG. 46C to the state shown in FIG. 46D. As the operator pulls guide element (990) proximally relative to handle (810), this pulls rack member (940) proximally within handle (810). The proximal movement of rack member (940) causes both pinions (914, 916) to spin freely or “freewheel” within handle (810). The corresponding proximal movement of guide element (990) also urges the distal tip of guide element (990) proximally relative to distal tip (886) of guide rail (884), such that the spatial relationship between the distal tip of guide element (990) and distal tip (886) of guide rail (884) may return to a relationship similar to that shown between distal tip (514) of guide element (510) and distal tip (502) of guide rail (500) in FIG. 28A. In addition, once rack member (940) returns to the proximal position shown in FIG. 46D, magnets (950, 952) may again magnetically couple with each other. The operator may understand that rack member (940) has fully returned to the proximal position shown in FIG. 46D when the operator feels a sudden increase in tension in guide element (990) while pulling on guide element (990).

To the extent that the operator did not observe transillumination effects indicating that the distal tip (886) of guide rail (884) was appropriately positioned at the targeted anatomical passageway when performing the steps described above with reference to the transition from the state shown in FIG. 46B to the state shown in FIGS. 46C, the operator may reposition shaft assembly (840) within the patient (P) and repeat the steps described above with reference to the transition from the state shown in FIG. 46B to the state shown in FIG. 46D until the operator confirms that guide rail (884) is appropriately positioned at the targeted anatomical passageway.

Once the operator has confirmed that guide rail (884) is appropriately positioned at the targeted anatomical passageway, the operator may continue to advance dilation catheter actuator (820) further distally relative to handle (810). During such advancement, dilation catheter actuator (820) reaches a second intermediate position as shown in FIG. 46E. With dilation catheter actuator (820) in the second intermediate position, rack member (920) is also in a second intermediate position. With rack member (920) at this second intermediate position, the teeth on the underside of rack member (920) begin to engage pinion (916). Thus, as the operator continues to advance dilation catheter actuator (820) further distally from the second intermediate position, the corresponding further distal advancement of rack member (920) causes pinions (914, 916) to rotate. The rotation of pinions (914, 916) causes rack member (940) to advance distally to the position shown in FIG. 46F. It should be understood that the distal forces imparted on rack member (940) by the rotation of pinions (914, 916), as driven by distal movement of dilation catheter actuator (820), may suffice to overcome the magnetic coupling between magnets (950, 952).

During the transition from the state shown in FIG. 46E to the state shown in FIG. 46F, guide element (990) may translate distally with dilation catheter assembly (860), with the distal tip of guide element (990) being maintained at the same longitudinal position as distal end (866) of dilation catheter assembly (860). This movement may thus correspond to the movement of dilation catheter (520) and guide element (510) described above with reference to the transition from the state shown in FIG. 28B to the state shown in FIG. 28C. As dilation catheter assembly (860) advances to the distal position associated with the state shown in FIG. 46F, balloon (864) enters the targeted anatomical passageway. Thus, after reaching the distal position shown in FIG. 46F, the operator may inflate balloon (864) to dilate the targeted anatomical passageway. In some cases, balloon (864) may be inflated and deflated repeatedly to achieve sufficient dilation. While balloon (864) is shown in the inflated state in FIG. 45, it should be understood that balloon (864) may in fact be in a deflated state during the stages of operation shown in FIGS. 46A-46F; and then be inflated after reaching the stage of operation shown in FIG. 46F.

When the dilation is complete, balloon (864) may be deflated and retracted proximally by proximal movement of dilation catheter actuator (820) relative to handle (810) as shown in the transition from the state shown in FIG. 46F to the state shown in FIG. 46G. Dilation catheter assembly (860) eventually reaches the position where distal end (866) of dilation catheter assembly (860) and the distal tip of guide element (990) both reach the same longitudinal position as distal tip (886) of guide rail (884). At this point, the lower teeth on rack member (920) disengages pinion (916), such that guide element (990) ceases further proximal movement while dilation catheter assembly (860) continues to translate proximally; and such that the distal tip of guide element (990) remains at distal tip (886) of guide rail (884). The operator continues to proximally retract dilation catheter actuator (820) along handle (810), as shown in the transition from the state shown in FIG. 46G to the state shown in FIG. 46H, to thereby return dilation catheter assembly (860) back to the proximal-most position. Guide element (990) may remain longitudinally stationary relative to handle (810) as dilation catheter assembly (860) translates proximally relative to handle (810) during the transition from the state shown in FIG. 46G to the state shown in FIG. 46H.

### B. Example of Guide Rail Adjustment Features

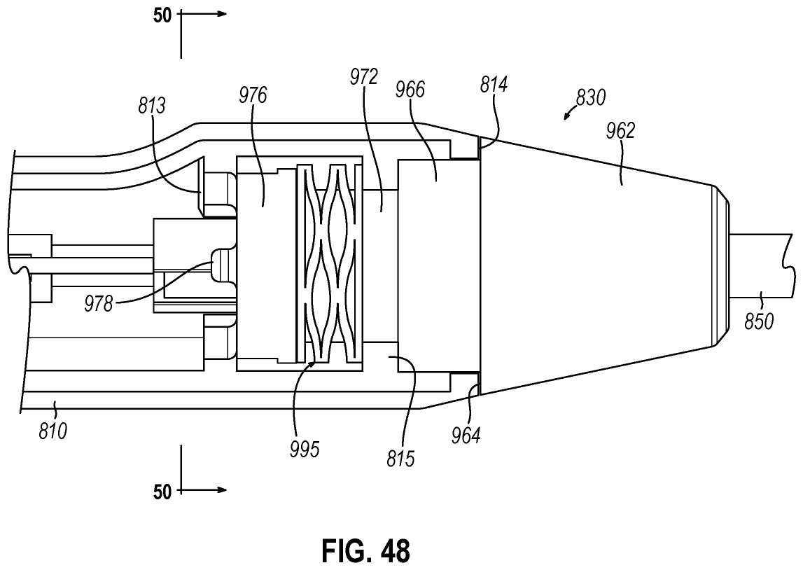

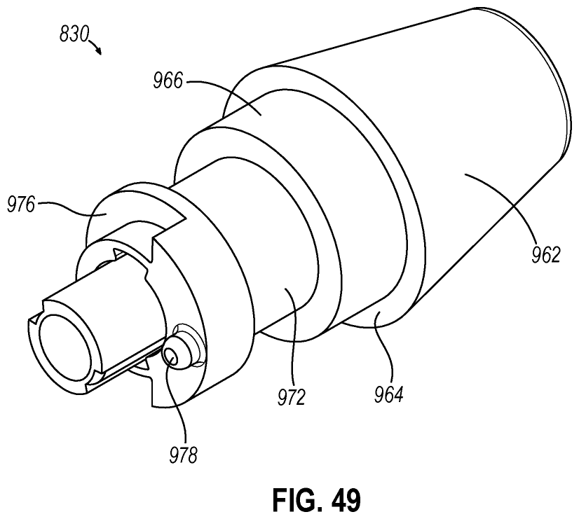

As noted above, guide rail actuation knob (830) may be rotated and translated relative to handle (810) to thereby rotate and translate guide rail assembly (880) and outer sheath (850) relative to handle (810). FIGS. 48-49 show guide rail actuation knob (830) of the present example in further detail. As best seen in FIG. 49, guide rail actuation knob (830) includes a distal nose portion (962) having a proximal face (964). A first cylindraceous portion (966) extends proximally relative to proximal face (964). A second cylindraceous portion (972), which has a smaller diameter than first cylindraceous portion (966), extends proximally relative to first cylindraceous portion (966). A flange portion (976) extends radially outwardly from second cylindraceous portion (972). A protrusion (978) extends proximally from flange portion (976).

As best seen in FIG. 48, guide rail actuation knob (830) is positioned in relation to handle (810) such that proximal face (964) abuts distal end (814); and such that distal nose portion (962) is distal to distal end (814). First cylindraceous portion (966) extends into handle (810) and abuts a first inner wall (815) in handle (810). Second cylindraceous portion (972) projects proximally through an opening formed in first inner wall (815). Flange portion (976) is positioned to abut a second inner all (813) in handle (810).

A wave spring assembly (995) is coaxially positioned about second cylindraceous portion (972) and is longitudinally interposed between flange portion (976) and first inner wall (815). It should be understood that wave spring assembly (995) is shown in a partially compressed state in FIG. 48; and that in the state of operation shown in FIG. 48, wave spring assembly (995) may in fact be in an expanded state such that wave spring assembly (995) simultaneously resiliently bears distally against first inner wall (815) and proximally against flange portion (976). Wave spring assembly (995) thus resiliently urges guide rail actuation knob (830) relative to handle (810) toward a proximal position. However, wave spring assembly (995) is compressible to allow distal movement of guide rail actuation knob (830) relative to handle (810) as described below.

As shown in FIGS. 50A-50C, second inner wall (813) includes three openings (992, 994, 996). Each opening (992, 994, 996) is sized to receive protrusion (978) of guide rail actuation knob (830). For instance, FIG. 50A shows guide rail actuation knob (830) in an angular position where protrusion (978) is disposed in opening (994) of second inner wall (813). For instance, FIG. 50B shows guide rail actuation knob (830) in an angular position where protrusion (978) is disposed in opening (992) of second inner wall (813). For instance, FIG. 50C shows guide rail actuation knob (830) in an angular position where protrusion (978) is disposed in opening (996) of second inner wall (813). It should be understood that the angular positions of openings (992, 994, 996) correspond to predetermined angular positions of guide rail assembly (880) and outer sheath (850) relative to handle (810), about the central longitudinal axis of shaft assembly (840).

FIGS. 51A-51D show an example of a sequence of operation for adjusting the angular position of guide rail assembly (880) relative to handle (810), about the central longitudinal axis of shaft assembly (840), via guide rail actuation knob (830). FIG. 51A shows guide rail actuation knob (830) in a proximal longitudinal position relative to handle (810); and at an angular position where protrusion (978) is disposed in opening (992) of second inner wall (813). Wave spring assembly (995) imparts a proximal bias resiliently urging guide rail actuation knob (830) toward this proximal longitudinal position. To adjust the angular position of guide rail assembly (880) relative to handle (810), the operator may simultaneously grasp handle (810) and guide rail actuation knob (830) and then push guide rail actuation knob (830) distally to the position shown in FIG. 51B. This distal movement of guide rail actuation knob (830) compresses wave spring assembly (995) and removes protrusion (978) from opening (992) of second inner wall (813).

While maintaining guide rail actuation knob (830) in the distal position, the operator rotates guide rail actuation knob (830) about the central longitudinal axis of shaft assembly (840), can be seen in the transition from the state shown in FIG. 51B to the state shown in FIG. 51C. In this example, the operator rotates guide rail actuation knob (830) 180 degrees, to thereby align protrusion with opening (996) of second inner wall (813). The operator then releases guide rail actuation knob (830), allowing wave spring assembly (995) to resiliently return guide rail actuation knob (830) to a proximal longitudinal position. This proximal movement of guide rail actuation knob (830) seats protrusion (978) in opening (996) of second inner wall (813), as shown in FIG. 51D. It should be understood that a similar process may be carried out to rotate guide rail actuation knob (830) to an angular position where protrusion (978) would be seated in opening (994) of second inner wall (813). It should also be understood that second inner wall (813) may include any suitable number of openings in any suitable angular positions. Moreover, flange portion (976) and second inner wall (813) may include any other suitable complementary structural features that provide different predetermined angular positions for guide rail actuation knob (830) and guide rail assembly (880).

### C. Example of Gripping Feature for Guide Element

As noted above, there may be scenarios where an operator wishes to independently advance guide element (990) through the targeted passageway (e.g., maxillary sinus ostium or frontal recess, etc.) and into the cavity (e.g., maxillary sinus cavity or frontal sinus cavity) to observe additional transillumination effects indicating successful passage of guide element (990) through the targeted passageway and into the cavity. Such independent advancement of guide element (990) may be performed after shaft assembly (840) is initially inserted into the patient (P), while dilation catheter actuator (820) and dilation catheter assembly (760) remain stationary in a partially advanced position. In the above description with reference to FIGS. 46B-46D, the operator may grasp a portion of guide element (990) protruding proximally from handle (810) and thereby push guide element (990) distally relative to handle (810) after dilation catheter actuator (820) and dilation catheter assembly (760) have been advanced distally enough to provide clearance for distal movement of rack member (940).

However, there may also be scenarios where the operator wishes to independently advance guide element (990) through the targeted passageway and into the cavity to observe additional transillumination effects indicating successful passage of guide element (990) through the targeted passageway and into the cavity before dilation catheter actuator (820) and dilation catheter assembly (760) have been advanced distally at all. In other words, the operator may wish to independently advance guide element (990) through the targeted passageway and into the cavity to observe additional transillumination effects indicating successful passage of guide element (990) through the targeted passageway and into the cavity while dilation catheter actuator (820) and dilation catheter assembly (760) are in the respective proximal positions shown in FIG. 46A. To that end, rather than providing a fixed, rigid coupling between guide element (990) and upright portion (944) of rack ember (940), a variation may provide a firm yet slidable coupling between guide element (990) and an upright portion of a rack member.

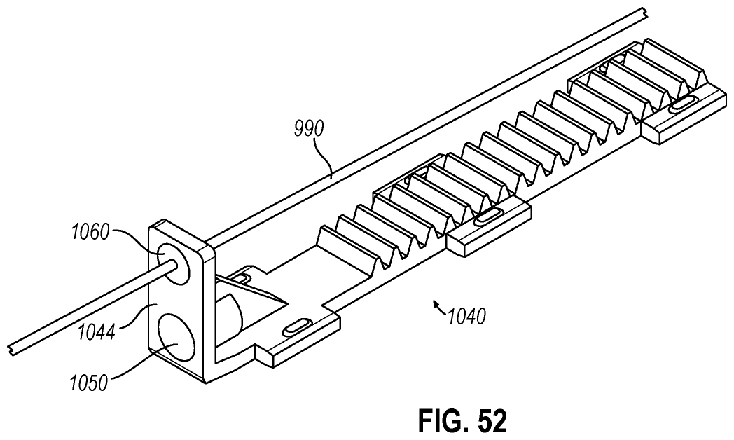

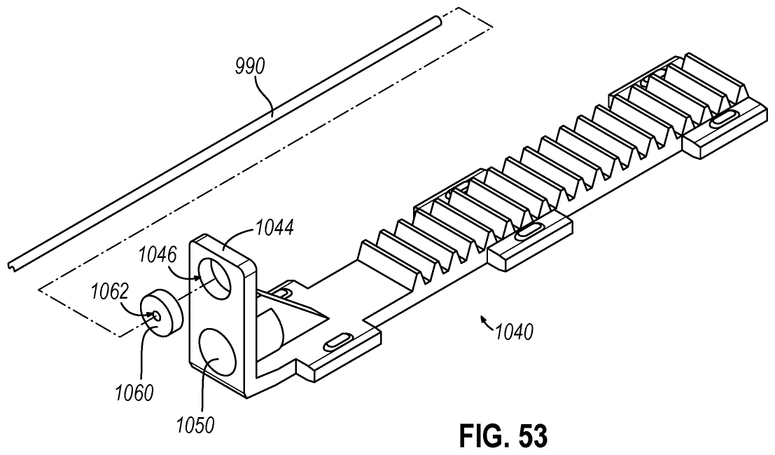

To that end, FIGS. 52-23 show an example of an alternative rack member (1040) that may be incorporated into dilation instrument (800) in place of rack member (940). Except as otherwise described below, rack member (1040) of this example may be configured and operable just like rack member (940) described above. Rack member (1040) of this example includes an integral permanent magnet (1050) like permanent magnet (950) of rack member (940). Rack member (1040) of this example also includes an upright portion (1044). However, unlike upright portion (944) of rack member (940), upright portion (1044) of rack member (1040) includes an opening (1046) with an elastomeric seal (1060) secured therein. By way of example only, elastomeric seal (1060) may comprise a disk or diaphragm of silicone or any other suitable material(s).

Elastomeric seal (1060) defines an opening (1062) having a diameter that is slightly smaller than the outer diameter of guide element (990). Since elastomeric seal (1060) is deformable, guide element (990) may be inserted into opening. However, elastomeric seal (1060) will resiliently bear inwardly against the outer diameter of guide element (990); and thereby provide substantial friction against guide element (990). This friction is sufficient for elastomeric seal (1060) to drive guide element (990) distally and proximally as rack member (1040) and elastomeric seal (1060) are driven distally and proximally in accordance with the teachings herein; yet this friction may also be overcome such that the operator may translate guide element (990) distally and proximally relative to elastomeric seal (1060) while rack member (1040) and elastomeric seal (1060) remain stationary.

In some versions, guide element (990) includes a marking to indicate guide element (990) being in a proximal-most position, such as at the stage of operation shown in FIG. 46A. Such a marking may be visible in relation to the proximal end of handle (810). In the event that the operator drives guide element (990) distally relative to rack member (1040) and elastomeric seal (1060) as described above, before dilation catheter actuator (820) and dilation catheter assembly (760) are advanced distally from the respective positions shown in FIG. 46A, the operator may observe the marking on guide element (990) in relation to handle (810) when the operator wishes to return guide element (990) back to the proximal position of FIG. 46A.

### VI. Examples of Reinforcement Features for Guide Elements

As noted above, a guide element (510, 990) may comprise a polymeric material, a metallic material, a tubular structure, a helical structure (e.g., similar to a guidewire, etc.), and/or any other suitable material(s) or configuration(s). In some versions, it may be desirable to provide additional column strength to a distal portion of guide element (510, 990). For instance, such additional column strength may tend to reduce the risk of the distal portion of guide element (510, 990) buckling as the distal portion of guide element (510, 990) is pushed through a small passageway such as a paranasal sinus ostium.

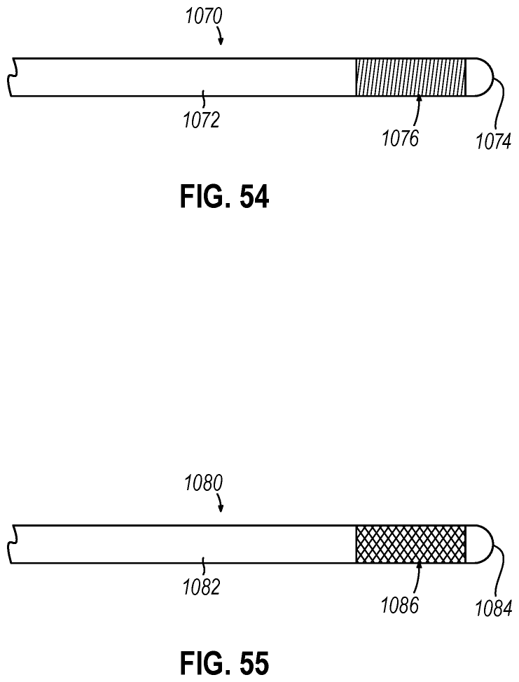

FIG. 54 shows an example of a guide element (1070) incorporating a reinforcement feature (1076) near a distal end (1074) of guide element (1070). Guide element (1070) may be configured and operable like any guide element (510, 990) described herein, except for the differences described below. Guide element (1070) of this example includes a polymeric shaft (1072) having substantial flexibility yet some tendency to buckle as distal end (1074) of shaft (1072) is pushed through a small passageway such as a paranasal sinus ostium. Reinforcement feature (1076) addresses this tendency by providing additional column strength to shaft (1072) near distal end (1074). Reinforcement feature (1076) of this example comprises a coil structure. Reinforcement feature (1076) may comprise metallic wire, a metallic band, or any other suitable material(s). Reinforcement feature (1076) may be fixedly secured about the exterior of shaft (1072). In addition, or in the alternative, reinforcement feature (1076) may be at least partially embedded within shaft (1072).

FIG. 55 shows another example of a guide element (1080) incorporating a reinforcement feature (1086) near a distal end (1084) of guide element (1080). Guide element (1080) may be configured and operable like any guide element (510, 990) described herein, except for the differences described below. Guide element (1080) of this example includes a polymeric shaft (1082) having substantial flexibility yet some tendency to buckle as distal end (1084) of shaft (1082) is pushed through a small passageway such as a paranasal sinus ostium. Reinforcement feature (1086) addresses this tendency by providing additional column strength to shaft (1082) near distal end (1084). Reinforcement feature (1086) of this example comprises a braid structure. Reinforcement feature (1086) may comprise metallic wires, metallic bands, polymeric fibers, or any other suitable material(s). Reinforcement feature (1086) may be fixedly secured about the exterior of shaft (1082). In addition, or in the alternative, reinforcement feature (1086) may be at least partially embedded within shaft (1082).

### VII. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a handle; (b) a guide member extending distally from the handle; (c) a dilation catheter slidably disposed relative to the guide member, the dilation catheter being operable to translate relative to the handle along a longitudinal range of motion from a proximal-most position to a distal-most position, the dilation catheter including: (i) an expandable element configured to dilate a passageway within a head of a patient, and (ii) a distal end; and (d) a dilation catheter actuator, the dilation catheter actuator comprising a rotary member operable to rotate relative to the handle to thereby drive translation of the dilation catheter along the longitudinal range of motion.

### Example 2

The apparatus of Example 1, the rotary member comprising a thumbwheel.

### Example 3

The apparatus of any of Examples 1 through 2, the rotary member comprising a pinion.

### Example 4

The apparatus of Example 3, further comprising a rack slidably disposed in the handle, the pinion being engaged with the rack such that the rotary member is rotatable to drive translation of the rack, the rack being configured to drive translation of the dilation catheter.

### Example 5

The apparatus of Example 4, further comprising a driver rod, the driver rod being interposed between the rack and the dilation catheter such that the rack is configured to drive translation of the dilation catheter via the driver rod.

### Example 6

The apparatus of any of Examples 4 through 5, further comprising an idler gear rotatably supported by the handle, the pinion being engaged with the rack via the idler gear.

### Example 7

The apparatus of any of Examples 1 through 6, a first portion of the rotary member being exposed relative to the handle, a second portion of the rotary member being positioned in an interior region of the handle.

### Example 8

The apparatus of any of Examples 1 through 7, the guide member comprising a guide rail having a distal end.

### Example 9

The apparatus of Example 8, the guide rail being slidably positioned within a lumen of the dilation catheter.

### Example 10

The apparatus of any of Examples 8 through 9, at least a portion of the guide rail being malleable.

### Example 11

The apparatus of any of Examples 8 through 10, the guide member and the dilation catheter being configured such that the distal end of the guide rail is distal to the distal end of the dilation catheter when the dilation catheter is in the proximal-most position, the guide member and the dilation catheter being further configured such that the distal end of the guide rail is proximal to the distal end of the dilation catheter when the dilation catheter is in the distal-most position.

### Example 12

The apparatus of any of Examples 1 through 11, further comprising a rigid sheath extending distally from the handle, the dilation catheter being positioned within the rigid sheath.

### Example 13

The apparatus of Example 12, the guide member being positioned within the dilation catheter.

### Example 14

The apparatus of any of Examples 1 through 13, the expandable element comprising a balloon.

### Example 15

The apparatus of any of Examples 1 through 14, the rotary member being configured for rotation via a plurality of strokes to thereby drive translation of the dilation catheter along the longitudinal range of motion.

### Example 16

An apparatus, comprising: (a) a handle, the handle having a first longitudinal end and a second longitudinal end, the first longitudinal end defining an opening; (b) a guide member extending distally from the handle; (c) a dilation catheter slidably disposed relative to the guide member, the dilation catheter being operable to translate relative to the handle along a longitudinal range of motion from a proximal-most position to a distal-most position, the dilation catheter including: (i) an expandable element configured to dilate a passageway within a head of a patient, and (ii) a distal end; and (d) a dilation catheter actuator, the dilation catheter actuator comprising: (i) a body operable to translate along an exterior of the handle to thereby drive translation of the dilation catheter along the longitudinal range of motion, and (ii) a longitudinally extending arm, the longitudinally extending arm being disposed in the opening of the first longitudinal end of the handle.

### Example 17

The apparatus of Example 16, the first longitudinal end being proximal to the second longitudinal end.

### Example 18

The apparatus of Example 17, the body having a distal end and a proximal end, the longitudinally extending arm extending distally from the proximal end of the body.

### Example 19

The apparatus of Example 16, the first longitudinal end being distal to the second longitudinal end.

### Example 20

The apparatus of Example 19, the body having a distal end and a proximal end, the longitudinally extending arm extending proximally from the distal end of the body.

### Example 21

The apparatus of any of Examples 16 through 20, the handle comprising an exterior rail, the dilation catheter actuator being slidably supported by the exterior rail.

### Example 22

The apparatus of Example 21, the body defining a slot, the exterior rail being slidably disposed in the slot.

### Example 23

The apparatus of any of Examples 21 through 22, the handle further comprising a stop at an end of the exterior rail, the stop being configured to arrest longitudinal movement of the dilation catheter actuator along the handle.

### Example 24

The apparatus of any of Examples 16 through 23, the longitudinally extending arm having a free end, the dilation catheter being fixedly secured relative to the free end of the longitudinally extending arm.

### Example 25

The apparatus of Example 24, further comprising a driver rod, the driver rod being interposed between the free end of the longitudinally extending arm and the dilation catheter such that the longitudinally extending arm is configured to drive translation of the dilation catheter via the driver rod.

### Example 26

The apparatus of Example 25, the free end of the longitudinally extending arm comprising a block defining an opening, the driver rod being fixedly secured within the opening of the block.

### Example 27

The apparatus of any of Examples 16 through 26, the handle having a length, the dilation catheter actuator having a length, the length of the dilation catheter actuator being at least half the length of the handle.

### Example 28

The apparatus of Example 27, the length of the dilation catheter actuator being at least 75% of the length of the handle.

### Example 29

The apparatus of Example 26, the length of the dilation catheter actuator being substantially equal to the length of the handle.

### Example 30

The apparatus of any of Examples 16 through 29, the guide member comprising a guide rail having a distal end.

### Example 31

The apparatus of Example 30, the guide rail being slidably positioned within a lumen of the dilation catheter.

### Example 32

The apparatus of any of Examples 30 through 31, at least a portion of the guide rail being malleable.

### Example 33

The apparatus of any of Examples 30 through 32, the guide member and the dilation catheter being configured such that the distal end of the guide rail is distal to the distal end of the dilation catheter when the dilation catheter is in the proximal-most position, the guide member and the dilation catheter being further configured such that the distal end of the guide rail is proximal to the distal end of the dilation catheter when the dilation catheter is in the distal-most position.

### Example 34

The apparatus of any of Examples 16 through 33, further comprising a rigid sheath extending distally from the handle, the dilation catheter being positioned within the rigid sheath.

### Example 35

The apparatus of Example 34, the guide member being positioned within the dilation catheter.

### Example 36

The apparatus of any of Examples 16 through 35, the expandable element comprising a balloon.

### Example 37

The apparatus of any of Examples 16 through 36, the rotary member being configured for rotation via a plurality of strokes to thereby drive translation of the dilation catheter along the longitudinal range of motion.

### Example 38

An apparatus, comprising: (a) a handle; (b) a guide member extending distally from the handle; (c) a dilation catheter slidably disposed relative to the guide member, the dilation catheter being operable to translate relative to the handle along a longitudinal range of motion from a proximal-most position to a distal-most position, the dilation catheter including: (i) an expandable element configured to dilate a passageway within a head of a patient, and (ii) a distal end; and (d) an actuation assembly, the actuation assembly comprising: (i) a slide configured to translate about an exterior of the handle, and (ii) a first rotary member operable to rotate relative to the handle, in response to translation of the slide about the exterior of the handle, to thereby drive translation of the dilation catheter along the longitudinal range of motion.

### Example 39

The apparatus of Example 38, the handle comprising one or more external rails, the slide being slidably supported by the one or more external rails.

### Example 40

The apparatus of any of Examples 38 through 39, the slide comprising an integral rack, the first rotary member comprising a pinion, the pinion being engaged with the integral rack.

### Example 41

The apparatus of Example 40, the handle defining an opening, a portion of the first rotary member protruding through the opening of the handle to engage the integral rack of the slide.

### Example 42

The apparatus of any of Examples 40 through 41, the integral rack being positioned exteriorly relative to the handle.

### Example 43

The apparatus of any of Examples 38 through 41, the actuation assembly further comprising a first rack member positioned within the handle, the dilation catheter being secured to the first rack member, the first rotary member being operable to drive translation of the first rack member to thereby drive translation of the dilation catheter along the longitudinal range of motion.

### Example 44

The apparatus of Example 43, further comprising a second rotary member interposed between the first rotary member and the first rack member, the second rotary member being configured to communicate motion from the first rotary member to the first rack member.

### Example 45

The apparatus of any of Examples 43 through 44, further comprising a guide element slidably disposed relative to the dilation catheter, the guide element including an indicator element, the indicator element being configured to indicate a position of the distal end of the second guide in three-dimensional space.

### Example 46

The apparatus of Example 45, the indicator element comprising one or more of a position sensor coil or an illuminating element.

### Example 47

The apparatus of any of Examples 45 through 46, the actuation assembly further comprising a translatable body slidably disposed in the handle, the guide element being secured to the translatable body, the translatable body being operable to drive translation of the guide element relative to the handle.

### Example 48

The apparatus of Example 47, the translatable body comprising a second rack member.

### Example 49

The apparatus of Example 48, the actuation assembly further comprising one or more pinions configured to couple the second rack member with the second rack member.

### Example 50

The apparatus of Example 48, the translatable body comprising a sled member with a resiliently biased cantilevered portion, the first rack member being configured to slidably engage the resiliently biased cantilevered portion.

### Example 51

The apparatus of any of Examples 47 through 50, the translatable body including a first retention feature, the handle including a second retention feature, the second retention feature being configured to cooperate with the first retention feature to resist translational movement of the translatable body relative to the handle.

### Example 52

The apparatus of Example 51, the second retention feature being configured to provide magnetic attraction with the first retention feature to resist translational movement of the translatable body relative to the handle.

### Example 53

The apparatus of Example 52, one of the first retention feature or the second retention feature comprising a permanent magnet.

### Example 54

The apparatus of Example 53, the other of the first retention feature or the second retention feature comprising a permanent magnet.

### Example 55

The apparatus of any of Examples 45 through 54, the actuation assembly being configured to provide distal translation of the dilation catheter relative to the handle while the guide element remains longitudinally stationary relative to the handle as the slide translates distally from a proximal position to an intermediate position relative to the handle.

### Example 56

The apparatus of Example 55, the actuation assembly being configured to provide simultaneous distal translation of the dilation catheter and the guide element relative to the handle as the slide translates distally from the intermediate position to a distal position relative to the handle.

### Example 57

The apparatus of Example 56, the actuation assembly being configured to provide simultaneous proximal translation of the dilation catheter and the guide element relative to the handle as the slide translates proximally from the distal position to the intermediate position relative to the handle.

### Example 58

The apparatus of Example 57, the actuation assembly being configured to provide proximal translation of the dilation catheter relative to the handle while the guide element remains longitudinally stationary relative to the handle as the slide translates proximally from the intermediate position to the proximal position relative to the handle.

### Example 59

An apparatus, comprising: (a) a handle having a proximal end and a distal end; (b) a guide rail extending distally from the body assembly, the guide rail having a malleable distal portion with a distal end, the guide rail having a proximal end that is positioned distally relative to the distal end of the handle; and (c) a dilation catheter assembly slidably disposed relative to the guide rail, the dilation catheter assembly including: (i) an expandable element configured to dilate a passageway within a head of a patient, and (ii) a distal end.

### Example 60

The apparatus of Example 59, further comprising a body fixedly secured to the proximal end of the guide rail.

### Example 61

The apparatus of Example 60, the body having a proximal end, the proximal end of the body being positioned distally relative to the distal end of the handle.

### Example 62

The apparatus of any of Examples 60 through 61, further comprising an outer sheath, the guide rail and the dilation catheter assembly being positioned within the outer sheath.

### Example 63

The apparatus of Example 62, the body being fixedly secured to the outer sheath.

### Example 64

The apparatus of any of Examples 62 through 63, the outer sheath having a proximal end, the proximal end of the body being positioned distally relative to the proximal end of the outer sheath.

### Example 65

The apparatus of any of Examples 59 through 64, further comprising a guide rail actuation assembly operable to drive movement of the guide rail relative to the handle.

### Example 66

The apparatus of Example 65, the guide rail actuation assembly being operable to drive angular movement of the guide rail relative to the handle.

### Example 67

The apparatus of any of Examples 65 through 66, the guide rail actuation assembly being operable to drive translational movement of the guide rail relative to the handle.

### Example 68

The apparatus of any of Examples 65 through 67, the guide rail actuation assembly comprising a knob, the knob being movable relative to the handle to thereby drive movement of the guide rail relative to the handle.

### Example 69

The apparatus of Example 68, the knob being positioned distally relative to the distal end of the handle.

### Example 70

The apparatus of any of Examples 68 through 69, the knob having a distal end, the proximal end of the guide rail being positioned distally relative to the distal end of the knob.

### Example 71

The apparatus of any of Examples 68 through 70, the knob having a first retention feature, the handle having a second retention feature, the first retention feature being configured to selectively engage the second retention feature to thereby retain the knob and the guide rail at a first angular position relative to the handle.

### Example 72

The apparatus of Example 71, the handle having a third retention feature, the first retention feature being configured to selectively engage the third retention feature to thereby retain the knob and the guide rail at a second angular position relative to the handle.

### Example 73

The apparatus of Example 72, the handle having a fourth retention feature, the first retention feature being configured to selectively engage the fourth retention feature to thereby retain the knob and the guide rail at a third angular position relative to the handle.

### Example 74

The apparatus of any of Examples 71 through 73, the first retention feature comprising a protrusion on a flange portion of the knob, the second retention feature comprising an opening formed in an inner wall of the handle.

### Example 75

The apparatus of any of Examples 71 through 74, the knob being translatable relative to the handle between a first longitudinal position and a second longitudinal position, the first retention feature being positioned to engage the second retention feature when the knob is in the first longitudinal position, the first retention feature being positioned to disengage the second retention feature when the knob is in the second longitudinal position.

### Example 76

The apparatus of Example 75, the knob being rotatable relative to the handle when the knob is in the second longitudinal position.

### Example 77

The apparatus of any of Examples 75 through 76, the second longitudinal position being distal to the first longitudinal position.

### Example 78

The apparatus of any of Examples 75 through 77, further comprising a resilient member configured to resiliently urge the knob toward the first longitudinal position.

### Example 79

The apparatus of Example 78, the resilient member comprising a wave spring assembly.

### Example 80

The apparatus of any of Examples 59 through 79, the guide rail having a distal region that includes the distal end and a proximal region that includes the proximal end, the distal region having a first Young's modulus value, the proximal region having a second Young's modulus value, the second Young's modulus value being different from the first Young's modulus value.

### Example 81

The apparatus of Example 80, the first Young's modulus value being less than the second Young's modulus value.

### Example 82

The apparatus of any of Examples 80 through 81, the first Young's modulus value ranging from approximately 20 N/mm² to approximately 70 N/mm².

### Example 83

The apparatus of any of Examples 80 through 81, the first Young's modulus value ranging from approximately 30 N/mm² to approximately 45 N/mm².

### Example 84

The apparatus of any of Examples 80 through 81, the first Young's modulus value being approximately 40 N/mm².

### Example 85

The apparatus of any of Examples 80 through 84, the guide rail further including an intermediate region between the distal region and the proximal region, the intermediate region having a Young's modulus value that progressively changes from the first Young's modulus value to the second Young's modulus value.

### Example 86

The apparatus of any of Examples 80 through 85, the distal region extending from the distal end of the guide rail to the distal end of the dilation catheter assembly.

### Example 87

The apparatus of Example 86, the distal region proximally terminating at the distal end of the dilation catheter assembly.

### Example 88

The apparatus of any of Examples 59 through 87, the guide rail comprising stainless steel.

### Example 89

The apparatus of any of Examples 1 through 15, the dilation catheter actuator further comprising a sliding member coupled with the handle, the sliding member being slidable along the handle to thereby drive rotation of the rotary member.

### Example 90

The apparatus of Example 89, the sliding member including an integral rack, the rotary member comprising a pinion, the integral rack being positioned in meshing engagement with the pinion of the rotary member.

### Example 91

The apparatus of Example 90, the handle defining a slot, the integral rack being positioned over the slot, a portion of the pinion protruding through the slot to engage the integral rack.

### VIII. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a handle;
(b) a guide member extending distally from the handle;
(c) a dilation catheter slidably disposed relative to the guide member, the dilation catheter being operable to translate relative to the handle along a longitudinal range of motion from a proximal-most position to a distal-most position, the dilation catheter including:
(i) an expandable element configured to dilate a passageway within a head of a patient, and
(ii) a distal end; and
(d) a dilation catheter actuator, the dilation catheter actuator comprising a rotary member operable to rotate relative to the handle to thereby drive translation of the dilation catheter along the longitudinal range of motion.

2. The apparatus of claim 1, the rotary member comprising a pinion.

3. The apparatus of claim 2, further comprising a rack slidably disposed in the handle, the pinion being engaged with the rack such that the rotary member is rotatable to drive translation of the rack, the rack being configured to drive translation of the dilation catheter.

4. The apparatus of claim 3, further comprising an idler gear rotatably supported by the handle, the pinion being engaged with the rack via the idler gear.

5. The apparatus of claim 1, a first portion of the rotary member being exposed relative to the handle, a second portion of the rotary member being positioned in an interior region of the handle.

6. The apparatus of claim 1, the guide member comprising a guide rail having a distal end.

7. The apparatus of claim 6, the guide rail being slidably positioned within a lumen of the dilation catheter.

8. The apparatus of claim 6, at least a portion of the guide rail being malleable.

9. The apparatus of claim 6, the guide member and the dilation catheter being configured such that the distal end of the guide rail is distal to the distal end of the dilation catheter when the dilation catheter is in the proximal-most position, the guide member and the dilation catheter being further configured such that the distal end of the guide rail is proximal to the distal end of the dilation catheter when the dilation catheter is in the distal-most position.

10. The apparatus of claim 1, further comprising a rigid sheath extending distally from the handle, the dilation catheter being positioned within the rigid sheath.

11. The apparatus of claim 10, the guide member being positioned within the dilation catheter.

12. The apparatus of claim 1, the expandable element comprising a balloon.

13. The apparatus of claim 1, the dilation catheter actuator further comprising a sliding member coupled with the handle, the sliding member being slidable along the handle to thereby drive rotation of the rotary member.

14. The apparatus of claim 13, the sliding member including an integral rack, the rotary member comprising a pinion, the integral rack being positioned in meshing engagement with the pinion of the rotary member.

15. The apparatus of claim 14, the handle defining a slot, the integral rack being positioned over the slot, a portion of the pinion protruding through the slot to engage the integral rack.

16. An apparatus, comprising:
(a) a handle;
(b) a guide member extending distally from the handle;
(c) a dilation catheter slidably disposed relative to the guide member, the dilation catheter being operable to translate relative to the handle along a longitudinal range of motion from a proximal-most position to a distal-most position, the dilation catheter including:
(i) an expandable element configured to dilate a passageway within a head of a patient, and
(ii) a distal end; and
(d) an actuation assembly, the actuation assembly comprising:
(i) a slide configured to translate about an exterior of the handle, and
(ii) a first rotary member operable to rotate relative to the handle, in response to translation of the slide about the exterior of the handle, to thereby drive translation of the dilation catheter along the longitudinal range of motion.

17. The apparatus of claim 16, further comprising a guide element slidably disposed relative to the dilation catheter, the guide element including an indicator element, the indicator element being configured to indicate a position of the distal end of the second guide in three-dimensional space;
the actuation assembly being configured to provide distal translation of the dilation catheter relative to the handle while the guide element remains longitudinally stationary relative to the handle as the slide translates distally from a proximal position to an intermediate position relative to the handle;
the actuation assembly being further configured to provide simultaneous distal translation of the dilation catheter and the guide element relative to the handle as the slide translates distally from the intermediate position to a distal position relative to the handle.

18. An apparatus, comprising:
(a) a handle having a proximal end and a distal end;
(b) a guide rail extending distally from the body assembly, the guide rail having a malleable distal portion with a distal end, the guide rail having a proximal end that is positioned distally relative to the distal end of the handle; and
(c) a dilation catheter assembly slidably disposed relative to the guide rail, the dilation catheter assembly including:
(i) an expandable element configured to dilate a passageway within a head of a patient, and
(ii) a distal end.

19. The apparatus of claim 18, further comprising:
(a) a body fixedly secured to the proximal end of the guide rail, the proximal end of the body being positioned distally relative to the distal end of the handle; and
(b) an outer sheath, the guide rail and the dilation catheter assembly being positioned within the outer sheath, the body being fixedly secured to the outer sheath, the outer sheath having a proximal end, the proximal end of the body being positioned distally relative to the proximal end of the outer sheath.

20. The apparatus of claim 18, further comprising a guide rail actuation assembly operable to drive movement of the guide rail relative to the handle.
